(19) United States
(12) Patent Application Publication
(10) Pub. No.: US 2026/0201336 A1
(43) Pub. Date: Jul. 16, 2026

(54) REPROGRAMMED SMOOTH MUSCLE CELLS AND METHODS RELATED THERETO

(71) Applicants: EMORY UNIVERSITY, Atlanta, GA (US); INDUSTRY-ACADEMIC COOPERATION FOUNDATION, YONSEI UNIVERSITY, Seoul (KR)

(72) Inventors: Young-sup YOON, Atlanta, GA (US); Kyung Hee KIM, Atlanta, GA (US); Ji Woong HAN, Atlanta, GA (US); Cholomi JUNG, Seoul (KR); Shin-Jeong LEE, Seoul (KR)

(73) Assignees: EMORY UNIVERSITY, Atlanta,, GA (US); INDUSTRY-ACADEMIC COOPERATION FOUNDATION, YONSEI UNIVERSITY, Seoul (KR)

(21) Appl. No.: 19/134,027

(22) PCT Filed: Nov. 29, 2023
(86) PCT No.: PCT/US2023/081603

§ 371 Date: May 29, 2025

Related U.S. Application Data
(60) Provisional application No. 63/428,597, filed on Nov. 29, 2022.

(51) Int. Cl. C12N 5/077; A61K 35/34; A61P 9/10; C07K 14/47

(52) U.S. Cl. CPC C12N 5/0661; A61K 35/34; A61P 9/10; C07K 14/4716; C12N 2500/38; C12N 2506/1307; C12N 2510/00

(57) ABSTRACT

Provided herein are novel reprogrammed smooth muscle cells (rSMCs) and methods of making and using the cells for the treatment of ischemia. The rSMCs are produced by culturing a fibroblast with an all-trans-retinoic acid (ATRA) under conditions that produce the rSMC from the fibroblast, wherein the fibroblasts are genetically modified to overexpress myocardin. The rSMCs offer advantages over currently available regenerative vascular therapies by promoting vascular perfusion in a recipient subject. In particular, the rSMCs can increase neovascularization of both small and large vessels.

### PRIOR RELATED APPLICATION

This application claims the benefit of U.S. Provisional Application No. 63/428,579 filed on Nov. 29, 2022 which is hereby incorporated by reference in its entirety.

### STATEMENT REGARDING FEDERALLY SPONSORED RESEARCH

This invention was made with government support under Grant Nos. HL150887 and HL157242 awarded by the National Institutes of Health. The government has certain rights in the invention.

### REFERENCE TO A SEQUENCE LISTING SUBMITTED AS XML VIA PATENT CENTER

This application contains a Sequence Listing in XML format. The Sequence Listing, named 043150-1413091.xml was created on Nov. 29, 2023, is 97 Kilobytes in size, and is hereby incorporated by reference in its entirety.

### BACKGROUND OF THE INVENTION

Ischemic cardiovascular disease is a leading cause of morbidity and mortality worldwide. Vascular insufficiency is a hallmark pathological feature of ischemic cardiovascular disease. Regenerative vascular therapies have been developed to promote neovascularization. However, such regenerative therapies are typically restricted to capillary-sized vessels. Additionally, regenerative therapies lack the ability to promote recruitment of mural cells (i.e., pericytes and vascular smooth muscle cells (SMCs)) to small or large vessels. Currently available therapies consequently lack the ability to effectively perfuse ischemic tissues and organs.

### SUMMARY

Provided herein is a method of producing a reprogrammed smooth muscle cell (rSMC) by culturing a fibroblast with an all-trans-retinoic acid (ATRA) under conditions that produce the rSMC from the fibroblast, wherein the fibroblasts are genetically modified to express or overexpress myocardin. The conditions that generate the rSMC from the fibroblast comprise contacting the fibroblast with the ATRA for at least two days (including, e.g., for 4-8 days). The fibroblast is optionally a mammalian fibroblast or, more specifically, a human fibroblast. For example, the genetically modified fibroblast is optionally a human dermal fibroblast.

The fibroblast can be genetically modified by any method known in the art to introduce into the fibroblast a heterologous nucleic acid that encodes myocardin. Such methods include but are not limited to viral transduction. Optionally, the heterologous nucleic acid is stably integrated into the fibroblast genome by, for example, gene editing.

Also provided herein are rSMCs or progeny thereof comprising a heterologous nucleic acid. The rSMC comprises a heterologous nucleic acid encoding myocardin and the rSMC co-expresses CNN1 and SMTN in a non-striated pattern. Optionally, the rSMC is prepared by any method described herein. The rSMCs contract by more than 10% in the presence of carbachol or another agent that promotes release of intracellular calcium.

A composition comprising a population of rSMCs and a pharmaceutically acceptable carrier is also provided. Such composition is designed for administration to a subject.

Also provided is a method of treating a subject with ischemia or at risk of developing ischemia by administering to the subject an effective amount of rSMCs or a composition comprising a population of rSMCs and a carrier. The effective amount of the rSMCs or composition increases vascular perfusion in the subject, increases neovascularization in the subject, and/or increases arteriogenesis in the subject.

### BRIEF DESCRIPTION OF THE DRAWINGS

The present application includes the following figures. The figures are intended to illustrate certain embodiments and/or features of the compositions and methods and to supplement any description(s) of the compositions and methods. The figures do not limit the scope of the compositions and methods, unless the written description expressly indicates that such is the case.

FIG. 1 is an exemplary schematic of the experimental design for treatment of human dermal fibroblasts (HDFs) with all-trans retinoic acid (ATRA) for four days, showing the increasing concentration of ATRA over the four day period.

FIG. 2 shows that STRA6 expression was increased in ATRA-treated HDFs compared to untreated HDFs with the increase more pronounced at higher concentrations of ATRA.

FIG. 3A-FIG. 3H show that the expression of eight genes specific to a contractile SMC phenotype (FIG. 3A, MYOCD; FIG. 3B, ACTA2; FIG. 3C, TAGLN; FIG. 3D, CALD1; FIG. 3E, DES; FIG. 3F, CNN1; FIG. 3G, MYH11; and FIG. 3H, SMTN) was increased in ATRA-treated HDFs compared to untreated HDFs. A concentration of 2 μM of ATRA was the most effective concentration tested for induction of SMC gene expression.

FIG. 4 shows that S100A4 expression decreased in ATRA-treated HDFs compared to untreated HDFs.

FIG. 5 is an exemplary schematic of the experimental design for generating MYOCD-transduced HDFs with ATRA treatment. HDFs were transduced with MYOCD at days 0 and 2 and then cultured with ATRA until day 16.

FIG. 6A-FIG. 6H show that expression of eight genes specific to a contractile SMC phenotype (FIG. 6A, MYOCD; FIG. 6B, ACTA2; FIG. 6C, TAGLN; FIG. 6D, CALD1; FIG. 6E, DES; FIG. 6F, CNN1; FIG. 6G, MYH11; and FIG. 6H, SMTN) increased in HDFs transduced with MYOCD and treated with ATRA (MATRA-treated HDFs) more than in untransduced HDF or MYOCD-treated HDF without ATRA treatment as shown in FIG. 5. Gene expression peaked on day 4 for all genes except for SMTN, which peaked on day 8.

FIG. 7 is an exemplary schematic of the experimental design for generating reprogrammed SMCs from HDFs. HDFs were transduced with MYOCD on days 0 and 2 and then cultured with ATRA until day 4.

FIG. 8A-FIG. 8H show that gene expression of eight genes specific to a contractile SMC phenotype (FIG. 8A, MYOCD; FIG. 8B, ACTA2; FIG. 8C, TAGLN; FIG. 8D, CALD1; FIG. 8E, DES; FIG. 8F, CNN1; FIG. 8G, MYH11; and FIG. 8H, SMTN) was significantly increased in MATRA-treated HDFs at day 4 as shown in FIG. 7, compared to untransduced HDFs and MYOCD-HDFs.

FIG. 9 shows that gene expression of STRA6 was significantly increased in MATRA-treated HDFs at day 4, compared to untransduced HDFs and MYOCD-HDFs without ATRA treatment.

FIG. 10A-FIG. 10C show that gene expression of Meta-VCL (FIG. 10A), TPM1 (FIG. 10B), and TPM2 (FIG. 10C), genes that regulate SMC contraction, was significantly increased in MATRA-treated HDFs at day 4, compared to untransduced HDFs and MYOCD-HDFs without ATRA treatment.

FIG. 11A-FIG. 11D show that gene expression of four genes specific to synthetic SMCs (KLF4 (FIG. 11A), MYH10 (FIG. 11B), MMP2 (FIG. 11C), and SPP1 (FIG. 11D)) was significantly reduced in MATRA-treated HDFs at day 4, compared to untransduced HDFs and MYOCD-HDFs without ATRA treatment.

FIG. 12 shows that gene expression of CSPG4, a pericyte gene, was increased in MATRA-treated HDFs at day 4, compared to untransduced HDFs. The gene expression of CSPG4 was higher in both MYOCD-HDFs and MATRA-treated HDFs, compared to untransduced HDFs.

FIG. 13A-FIG. 13D show that gene expression of S100A4 (FIG. 13A), THY1 (FIG. 13B), VIM (FIG. 13C), and PDGFRA (FIG. 13D), genes representative of fibroblasts, was decreased in MATRA-treated HDFs at day 4, compared to untransduced HDFs and MYOCD-HDFs.

FIG. 14 shows flow cytometry analysis for ACTA2 expression in HDFs, MYOCD-HDFs, and MATRA-treated HDFs. At day 4, approximately 57% of MATRA-treated HDFs expressed ACTA2, compared to approximately 5.0% and 35.3% of the cells in treatment groups for the untransduced HDF and MYOCD-HDF without ATRA, respectively.

FIG. 15 shows flow cytometry analysis for MYH11 expression in HDFs, MYOCD-HDFs, and MATRA-treated HDFs. At day 4, MATRA-treated HDFs exhibited MYH11 in approximately 48% of cells, compared to approximately 3.6% and 10.2% of the cells in the untransduced HDF and MYOCD-HDF groups, respectively.

FIG. 16 shows the flow cytometry gating strategy for selection of single live cells in the MATRA-treated HDF, MYOCD-HDF, and untransduced HDF groups.

FIG. 17 shows the flow cytometry gating strategy for identification of ACTA2+ cells in the MATRA-treated HDF, MYOCD-HDF, and untransduced HDF groups.

FIG. 18 shows the flow cytometry gating strategy for identification of MYH11+ cells in the MATRA-treated HDF, MYOCD-HDF, and untransduced HDF groups.

FIG. 19 shows photomicrographs of untransduced HDFs, MYCOD-HDFs, and MATRA-treated HDFs using double immunofluorescence staining for contractile SMC markers, ACTA2 (first column) and TAGLN (second column), with DAPI (middle column) as a nuclear counterstain. The two right columns show merged staining for TAGLN, DAPI, and ACTA2. The boxed field in the fourth column is shown at further magnification in the fifth column. MATRA-treated HDFs robustly co-expressed ACTA2 and TAGLN compared to MYOCD-HDFs and untransduced HDFs.

FIG. 20 shows photomicrographs of untransduced HDFs, MYCOD-HDFs, and MATRA-treated HDFs using double immunofluorescence staining for CNN1 (first column) and SMTN (second column), with DAPI (third column) as a nuclear counterstain. The two right columns show merged staining for all three. The boxed field is shown at further magnification in the far-right column. MATRA-treated HDFs robustly expressed CNN1 and SMTN compared to MYOCD-HDFs and untransduced HDFs.

FIG. 21 shows confocal microscopic images (left) of untransduced HDFs, MYOCD-HDFs, MATRA-treated HDFs, and human aortic SMCs (HAoSMCs) and a graph (right) showing the change in surface area of the cells before and after carbachol treatment (dashed line in bottom row indicating the contracted states and the solid line indicated the uncontracted state). MATRA-treated HDFs contracted by approximately 28%, measured by cell surface area, in the presence of carbachol. HAoSMCs contracted by approximately 32%, whereas untransduced HDFs contracted by approximately 9% and MYOCD-HDFs contracted by approximately 20%.

FIG. 22 shows photographs of collagen matrices (left) harboring untransduced HDFs, MYOCD-HDFs, MATRA-treated HDFs, and HAoSMCs and a graph (right) showing the change in surface area of the cells two days after treatment with carbochol (dashed line). MYOCD-HDFs and MATRA-treated HDFs displayed a stronger contraction in the presence of carbachol than did HAoSMCs and untransduced HDFs in collagen gels.

FIG. 23 shows confocal microscopic images of Fluo-4 (GFP) preloaded untransduced HDFs, MYOCD-HDFs, MATRA-treated HDFs, and HAoSMCs before and after treatment with carbachol. In the presence of carbachol, MATRA-treated HDFs released a similar amount of intracellular calcium as HAoSMCs, shown by fluorescence intensity in the bottom panels for each group. Both MATRA-treated HDFs and HAoSMCs displayed more intracellular calcium release than untransduced HDFs and MYOCD-HDFs.

FIG. 24 shows a graph of the percentage of Fluo-4 preloaded cells that responded to carbachol. After carbachol treatment, MATRA-treated HDFs had a significantly higher intracellular calcium release response rate compared to HAoSMCs, MYOCD-HDFs, and untransduced HDFs.

FIG. 25 is an exemplary schematic of parameters (primary peak, time to maximum fluorescence (F/F₀), F/F₀ max, and multiple peaks) used to assess intracellular calcium release.

FIG. 26 shows that the maximum fluorescence intensity (F/F₀ max) indicating the magnitude of intracellular calcium release with carbachol treatment as shown in FIG. 25. Calcium release was significantly higher in MATRA-treated HDFs as compared to HAoSMCs, MYOCD-HDFs, and untransduced HDFs.

FIG. 27 shows the time to achieve F/F₀ max, with fluorescence indicating the time to maximum intracellular calcium release as shown in FIG. 25. The time to F/F₀ max was significantly shorter for MATRA-treated HDFs, MYOCD-HDFs, and HAoSMCs as compared to untransduced HDFs.

FIG. 28 shows overlaid graphs of F/F₀ over 550 seconds from five randomly selected untransduced HDFs treated with carbachol. Untransduced HDFs produced one or no primary peak of intracellular calcium release in response to carbachol treatment.

FIG. 29 shows overlaid graphs of F/F₀ over 550 seconds from five randomly selected MYOCD-HDFs treated with carbachol. MYOCD-HDFs produced one large primary peak of intracellular calcium release, followed by a low number of recurrent peaks, in response to carbachol treatment.

FIG. 30 shows overlaid graphs of F/F₀ over 550 seconds from five randomly selected MATRA-treated HDFs (labelled rSMCs) in response to carbachol treatment. MATRA-treated HDFs produced a more robust primary peak of intracellular calcium release, followed by recurrent peaks over an extended period, in response to carbachol treatment.

FIG. 31 shows overlaid graphs of F/F₀ over 550 seconds from five randomly selected HAoSMCs in response to carbachol treatment. HAoSMCs produced one large primary peak of intracellular calcium release, followed by a low number of recurrent peaks, in response to carbachol treatment.

FIG. 32 shows overlaid graphs of F/F₀ for calcium release from a single untransduced HDF, MYOCD-HDF, MATRA-treated HDF, and HAoSMC in response to carbachol. The MATRA-treated HDF produced a robust primary peak of intracellular calcium release, followed by propagated waves of calcium release events over the test period, in response to carbachol treatment. However, the MYOCD-HDF and HAoSMC each produced a large primary peak followed by a low number of recurrent peaks, and the untransduced HDF produced no primary peak.

FIG. 33 shows phase-contrast microscopic images before and 24 hours after a scratch wound healing assay was performed and a graph showing the percentage of wound healing in each treatment group. Significantly fewer MATRA-treated HDFs and HAoSMCs migrated into a scratch wound (designated by dashed lines in the images) than did untransduced and MYOCD-HDFs.

FIG. 34 shows transmission electron microscopic images of untransduced HDFs, MYOCD-HDFs, and MATRA-treated HDF. The boxed field in the left column are further magnified in the right column and certain cellular elements are labeled, MATRA-treated HDFs had more contractile filaments anchored by dense bodies (DB) but substantially fewer mitochondria (M) and free ribosomes, compared to MYOCD-HDFs and untransduced HDFs.

FIG. 35 shows the results of principal component analysis for four distinct groups of gene expression in MATRA-treated HDFs (designated rSMCs), HAoSMCs, MYOCD-HDFs, and untransduced HDFs assessed with RNA sequencing data.

FIG. 36 shows a cluster heatmap showing the correlation between HDFs, MYOCD-HDFs, MATRA-treated rSMCs, and HAoSMCs by the Pearson correlation coefficient method. The heatmap shows four distinct groups of gene expression by cell type. Low correlation coefficients are shown in dark gray and high correlation coefficient values are shown in light gray.

FIG. 37 shows variation plots of principal component analysis of RNA sequencing data. MATRA-treated cells (rSMCs) displayed different gene expression patterns than HAoSMCs, MYOCD-HDFs, and untransduced HDFs and variations among replicates were minimal.

FIG. 38 shows a cluster heatmap of gene expression across cell types. Low correlation coefficients are shown in dark gray and high correlation coefficient values are shown in light gray. rSMCs displayed similar gene expression patterns to HAoSMCs but varied from gene expression patterns in MYOCD-HDFs and untransduced HDFs, assessed by principal component analysis of RNA sequencing data.

FIG. 39 shows a Venn diagram demonstrating the numbers and overlaps of differentially expressed genes (DEGs) among the groups. DEGs were sorted by relative expression to untransduced HDFs. rSMCs displayed high overlap of differentially expressed genes with HAoSMCs, compared with MYOCD-HDFs.

FIG. 40A-FIG. 40D show Mfuzz clusters demonstrating up-regulated gene expression patterns for each group (FIG. 40A, HDF; FIG. 40B, MYOCD-HDF; FIG. 40C, rSMC; FIG. 40D, HAoSMC). A subset of the most regulated genes for each group are shown. The Gene Ontology (GO) terms are shown. BP is biological process, CO is cellular component, HP is human phenotype, and MF is molecular function. Highly expressed genes for HDFs relate to mitosis and cell proliferation (KIF4A, CDCA8, CENPA, and GTSE1); highly expressed genes for MYOCD-HDFs include endolysosomal genes (LYPLA2, TPP1, and TANGO2) and cytoskeletal genes (EPS8L2 and SYNE3); highly expressed genes for rSMCs include muscle development and function genes (SORBS1, CNN1, and MYL7) and embryonic skeletal system morphogenesis genes (HOXD13 and HOXC13); and highly expressed genes for HAoSMCs include signal transduction system genes (MRAP2, SULTIE1, CACNG8, and CD200).

FIG. 41A-FIG. 41D show Mfuzz clusters demonstrating down-regulated gene expression patterns for each group (FIG. 41A, HDF; FIG. 41B, MYOCD-HDF; FIG. 41C, rSMC; FIG. 41D, HAoSMC). The GO terms are as described for FIG. 40. Downregulated gene expression for MYOCD-HDFs related to biological processes; rSMCs downregulated gene expression for genes related to cellular components; and HAoSMCs downregulated gene expression for genes related to the human phenotype. The number of downregulated genes ranged from 632 to 1,013 across the groups.

FIG. 42A-FIG. 42J show graphs of expression levels (shown as Log 2-counts per million mapped reads (Log 2 CPMs)) of ten genes specific to a SMC phenotype (FIG. 42A, ACTA2; FIG. 42B, CALD1; FIG. 42C, CNN1; FIG. 42D, MYH11; FIG. 42E, MYL6; FIG. 42F, MYOCD; FIG. 42G, TAGLN; FIG. 42H, TPM1; FIG. 42I, TPM2; and FIG. 42J, VCL). Expression was substantially higher in MATRA-treated HDFs (rSMCs) than in HAoSMCs, MYOCD-HDFs, and untransduced HDFs.

FIG. 43 shows a volcano plot demonstrating the differential expression of genes in untransduced HDFs as compared to rSMCs.

FIG. 44 shows a tree plot demonstrating the enriched GO terms in rSMCs. Genes were sorted by relative expression as compared to HDF expression. rSMCs were enriched in expression of genes for contraction and muscle development, as compared to gene expression in untransduced HDFs.

FIG. 45 shows a network plot of genes by gene ontology terms for tissue morphogenesis, skeletal system development, striated muscle tissue development, muscle tissue development, and cardiac muscle tissue development. Dot size for each term (as shown in FIG. 46) indicates the number of genes evaluated for that term, with the smallest dot representing 20 genes and the largest representing 60 genes.

FIG. 46 shows a network plot of genes by gene ontology terms for tissue morphogenesis, striated muscle tissue development, muscle tissue development, and cardiac muscle tissue development. Dot size for each term indicates the number of genes evaluated for that term, with the smallest dot representing 20 genes and the largest representing 60 genes.

FIG. 47A-FIG. 47B show a Quantitative Set Analysis of Gene Expression (QuSAGE) plot (FIG. 47A) and expression of genes related to REACTOME_SMOOTH_MUSCLE_CONTRACTION (FIG. 47B). Expression of genes related to smooth muscle contraction were significantly enriched in MATRA-treated HDFs (rSMCs) compared to untransduced HDFs.

FIG. 48A-FIG. 48D show a GuSAGE plot (FIGS. 48A and 48C) and REACTOME_SMOOTH_MUSCLE_CONTRACTION (FIGS. 48B and 48D) comparing bulk RNA-sequence in MYOCD-HDFs as compared to untransduced HDFs (FIGS. 48A-B) or as compared MATRA-treated HDFs (rSMCs) (FIGS. 48C-D).

FIG. 49A-FIG. 49F show Gene Set Enrichment Analysis (GSEA) plots demonstrating the enriched gene sets associated with SMCs in MATRA-treated HDFs (rSMCs). Genes for muscle system process (FIG. 49A), muscle contraction (FIG. 49B), muscle cell development (FIG. 49C), contractile fiber (FIG. 49D), actin cytoskeleton (FIG. 49E), actin binding (FIG. 49F) were sorted by relative expression to HDFs. The presence of gene signature is shown by black vertical lines in the bottom of each graph. Normalized enrichment score (NES); statistical significance as false discovery rate (FDR).

FIG. 50A-FIG. 50H show GSEA plots for cardiomyocyte (CM) genes (e.g., genes related to cardiac cell development (FIG. 50A), positive regulation of heart contraction (FIG. 50B), actinin binding (FIG. 50C), alpha actinin binding (FIG. 50D), sarcomere organization (FIG. 50E), sarcoplasm (FIG. 50F), sarcoplasmic reticulum membrane (FIG. 50G), and I band (FIG. 50H)) in rSMCs as compared to untransduced HDFs.

FIG. 51A-FIG. 51F show graphs of qRT-PCR analysis of six genes specific to CMs (TNNI1 (FIG. 51A), TNNT2 (FIG. 51B), TNNI3 (FIG. 51C), MYH6 (FIG. 51D), ACTN2 (FIG. 51E), and MYH7 (FIG. 51F)). Expression of each gene was reduced in rSMCs, MYOCD-HDFs, and untransduced HDFs compared to human embryonic stem cell-derived cardiomyocytes (hESC-derived CM) at day 30. GADPH was used for normalization.

FIG. 52A and FIG. 52B show laser Doppler perfusion images (LDPI) (FIG. 52A) and graphs of the perfusion ratio (FIG. 52B) from murine models of hindlimb ischemia (HLI) taken at various time points without treatment (HLI) and treatment with untransduced HDFs (HLI+HDF), MYOCD-HDFs (HLI+MYOCD-HDFs) or MATRA HDFs (HLI+rSMC). Left leg, non-ischemic; right leg, ischemic; lighter gray, high LDPI index; darker gray, low LDPI index. In the graph, perfusion ratios depicted from bottom to top are HLI, HLI+HDF, HLI+MYOCD-HDFs, and HLI+rSMC. The images and data show that rSMCs treatment enhances blood flow in an ischemic limb.

FIG. 53 shows confocal microscopic images of longitudinally sectioned ischemic limbs perfused with labeled isolectin B4 (ILB4) and harvested at day 28 post-HLI. rSMCs enhanced blood flow into hindlimb muscle in a murine model of hindlimb ischemia, compared to untreated or treatment with MYOCD-HDFs or untransduced HDFs. Fluorescence intensity signifies perfusion of fluorescein-conjugated ILB4 perfusion into hindlimb muscle vessels.

FIG. 54 shows a confocal image of ILB4-perfused ischemic hindlimbs harvested at day 28 post-surgery (top left), and its resulting image after running the AngioTool software (top right) following human rSMC transplantation. The bottom image is a magnification of the AngioTool image showing vessel surfaces, vascular structures, small non-vascular particles, and branching points.

FIG. 55 shows the average vessel density in murine ischemia model treatment groups as described above for FIG. 52. Treatment with rSMCs significantly increased the average vessel density compared to treatment with MYOCD-HDFs or untransduced HDFs. Vessel density is measured as percent vessel area divided by explant area.

FIG. 56 shows average vessel density in murine ischemia model treatment groups following human rSMC transplantation. In the murine model of hindlimb ischemia, treatment with human rSMCs significantly increased vessel density compared to treatment with MYOCD-HDFs or untransduced HDFs. Vessel density is measured as described for FIG. 55.

FIG. 57 shows the average total length of vessels for the treatment groups as described for FIG. 52. Treatment with rSMCs significantly increased the average total length of vessels compared to treatment with MYOCD-HDFs or untransduced HDFs.

FIG. 58 shows the average total number of end points for the treatment groups as described for FIG. 52. Treatment with rSMCs significantly increased the average total number of vessel endpoints compared to treatment with MYOCD-HDFs or untransduced HDFs.

FIG. 59 shows the total number of junctions for the treatment groups as described for FIG. 52. Treatment with rSMCs significantly increased the average total number of vessel junctions compared to treatment with MYOCD-HDFs or untransduced HDFs.

FIG. 60 shows the average junction density for the treatment groups as described for FIG. 52. Treatment with rSMCs significantly increased the average vessel junction density compared to treatment with MYOCD-HDFs or untransduced HDFs.

FIG. 61 shows the total lengths of vessels in murine ischemia model treatment groups following human rSMC transplantation. Treatment with rSMCs significantly increased the vessel density compared to treatment with MYOCD-HDFs or untransduced HDFs.

FIG. 62 shows the total number of end points in murine ischemia model treatment groups following human rSMC transplantation. Treatment with rSMCs significantly increased the total number of endpoints compared to treatment with MYOCD-HDFs or untransduced HDFs.

FIG. 63 shows the total number of vessel junctions in murine ischemia model treatment groups following human rSMC transplantation. Treatment with rSMCs significantly increased the total number of vessel junctions compared to treatment with MYOCD-HDFs or untransduced HDFs.

FIG. 64 shows the junction density in murine ischemia model treatment groups following human rSMC transplantation. Treatment with rSMCs significantly increased the vessel junction density compared to treatment with MYOCD-HDFs or untransduced HDFs.

FIG. 65 shows the limb loss score for treatment groups as described in FIG. 52. The limb loss score was calculated by the following scoring for assessment of ischemic hindlimb damage: 0=no necrosis; 1=tip necrosis; 2=toe necrosis; 3=foot necrosis; 4=leg necrosis; and 5=whole limb loss. Treatment with rSMCs significantly decreased the limb loss score compared to treatment with MYOCD-HDFs or untransduced HDFs.

FIG. 66 shows photographs of hindlimbs of representative animals with ischemic left hindlimbs from murine ischemia model treatment groups following human rSMC transplantation. Treatment with rSMCs significantly decreased loss or necrosis of mouse hindlimbs compared to treatment with MYOCD-HDFs or untransduced HDFs.

FIG. 67 shows the strategy used to differentiate lymphatic and blood vessels determine functional endothelium with systemic injection of fluorescein-conjugated-ILB4. Immunofluorescence staining for LYVE1 was used in the ILB4-perfused ischemic hindlimbs harvested at day 28 post-surgery. The magnified images show the restricted expression of ILB4 only in blood vessels, not ILB4 LYVE1⁺ lymphatic vessels. DAPI was used as a nuclear counterstain.

FIG. 68 is a schematic showing the sectioning strategy for visualizing a cross section of an ischemic hindlimb following intramuscular transplantation of pre-labeled CM-DiI rSMCs and harvested at 28 days post-surgery.

FIG. 69 shows confocal microscopic images of cross sections of ischemic hindlimb as acquired according to FIG. 68. Arrows indicate ILB4⁺ labelled capillaries surrounded by DiI⁺ rSMCs.

FIG. 70 is a schematic showing the sectioning strategy for visualizing a longitudinal section of an ischemic hindlimb following intramuscular transplantation of pre-labeled CM-DiI rSMCs and harvested at 28 days post-surgery.

FIG. 71 shows confocal microscopic images of longitudinal sections of ischemic hindlimb as acquired according to FIG. 70. rSMCs (arrows) indicate DiI⁺ rSMCs associated with ILB4⁺ vessels.

FIG. 72 shows ACTA2 immunofluorescence staining in cross section of ILB4⁺ arteriolar vessels. Arrows in the bottom left panel indicate ILB4⁺ vessels surrounded by DiI+ rSMCs or the investment of DiI⁺ rSMCs to ILB4⁺ arteriolar vessels. ACTA2⁺ rSMCs localize around the circumference of ILB4⁺ arteriolar vessels (curved dashed line in upper right panel).

FIG. 73 shows immunofluorescence staining for SMTN in a longitudinal section of a large ILB4⁺ vessel. Arrows in the bottom left panel indicate ILB4⁺ vessels surrounded by DiI⁺ rSMCs, and investment of DiI⁺ rSMCs to a large ILB4⁻ vessel. DiI⁺ rSMCs express SMTN and localize in vascular walls of vessels approximately 35 μm in diameter.

FIG. 74 shows a 3D rendering of the large ILB4⁺ vessel composed of immunofluorescently labelled SMTN⁺ DiI⁺ rSMCs.

FIG. 75 shows confocal microscopic images of DiI⁺ HDFs in cross sections of the ILB4-perfused hindlimbs harvested at day 28. DiI-pre-labelled HDFs were intramuscularly injected into three sites of ischemic hindlimbs.

FIG. 76 shows immunofluorescence staining for ACTA2 in longitudinal sections of ILB4⁺ vessels. The magnified images show the contribution of DiI⁺ HDFs to ILB4⁺ capillaries, not ILB4⁺ large vessels. ACTA2⁺ HDFs localize around the circumference of ILB4⁺ capillaries, not large vessels.

FIG. 77 shows immunofluorescence staining for SMTN in longitudinal sections of ILB4⁺ vessels. DiI-pre-labelled rSMCs were intramuscularly injected into three sites of ischemic hindlimbs. The magnified images show the investment of SMTN⁺DiI⁺ rSMCs (arrows) to ILB4⁺ capillaries.

FIG. 78 shows immunofluorescence staining for SMTN in longitudinal sections of ILB4⁺ vessels. DiI-pre-labelled rSMCs were intramuscularly injected into three sites of ischemic hindlimbs. The magnified images show the investment of SMTN⁺DiI⁺ rSMCs (arrows) to ILB4⁺ larger vessels.

FIG. 79 is a schematic of the experimental design for evaluating the effect of rSMCs on vascular permeability. Human umbilical vein ECs (HUVECs) and rSMCs were seeded onto opposite sides of a semi-porous membrane and the membrane was diffused with FITC-dextran.

FIG. 80 shows cross sectional views of FITC-dextran diffusion across the HUVEC monolayer seeded onto the luminal side or a co-culture model with HUVECs and human cells (HDF and rSMCs) seeded onto the abluminal side of a 24-well Transwell insert.

FIG. 81 shows immunofluorescence staining (left) for PECAM1 and CNN1 in the HUVEC monolayer or co-culture model with HUVECs and human cells and quantitative analysis (right) of the FITC-dextran permeability in the HUVEC monolayer or co-culture model with HUVECs and human cells.

FIG. 82A-FIG. 82J show qRT-PCR analyses of the expression of four genes specific to angiogenesis (Angpt1 (FIG. 82A), Fgf2 (FIG. 82B), VEGFA (FIG. 82C), and Hif1a (FIG. 82D)), eight genes specific to arteriogenesis (Ccl2 (FIG. 82E), Ccr2 (FIG. 82F), Tgfb1 (FIG. 82G), PDGFB (FIG. 82H), Csf1 (FIG. 82I), Mmp2 (FIG. 82J), MMP3 (FIG. 82K), Mmp9 ((FIG. 82L)), two genes specific to Notch signaling pathway (Hey1 (FIG. 82M) and Dil4 (FIG. 82N)), and two genes specific to SMC engraftment and vascular function (Igf1 (FIG. 82O) and Hgf (FIG. 82P)) in non- and ischemic tissues (HLI, HLI+HDF, and HLI+rSMC) harvested at day 7 post-transplantation. Gapdh was used as a housekeeping gene and for normalization.

### DETAILED DESCRIPTION

Provided herein is a method of producing a reprogrammed smooth muscle cell (rSMC). Also provided is a rSMC and a composition comprising a population of rSMCs. Also provided is a method of treating ischemia with the composition described herein in a subject in need thereof. The rSMCs and compositions thereof overcome limitations of currently available methods for treating ischemia. The rSMCs or compositions thereof promote neovascularization of capillaries as well as neovascularization of larger vessels like arteries and arterioles and also promotes recruitment of mural cells to capillaries and larger vessels. Additionally, the rSMCs or compositions thereof can be used to effectively promote perfusion of ischemic tissues and organs.

### Methods of Producing Reprogrammed Smooth Muscle Cells

Provided herein is a method of producing a reprogrammed smooth muscle cell (rSMC; used synonymously herein with MATRA-treated fibroblast). The method comprises culturing a fibroblast with an all-trans-retinoic-acid (ATRA) under conditions that produce a rSMC from the fibroblast, wherein the fibroblasts are genetically modified to overexpress myocardin.

As used throughout, a rSMC refers to a cell generated from a fibroblast using the methods described herein. The rSMCs, also referred to herein as MATRA-treated HDFs, have certain biomarkers and functions exemplified by naturally occurring smooth muscle cells. For example, the rSMC cell co-expresses CNN1 and SMTN in a non-striated pattern and contracts in the presence of intracellular calcium. However, unlike a naturally occurring smooth muscle cell, a rSMC comprises a heterologous nucleic acid sequence encoding myocardin.

Fibroblasts used to produce the rSMCs can be mammalian fibroblasts, including, for example, human fibroblasts. Optionally the fibroblast is a post-natal fibroblast, including neonatal or adult fibroblasts. Optionally, the fibroblast used to produce an rSMC is a human dermal fibroblast. The fibroblasts are optionally from the same subject as a subject to be treated to be treated or from a different subject.

As used throughout, ATRA refers to (2E,4E,6E,8E)-3,7-dimethyl-9-(2,6,6-trimethylcyclohexen-1-yl) nona-2,4,6,8-tetraenoic acid.

Optionally, the conditions that generate one or more rSMCs from one or more genetically modified fibroblasts include culturing the fibroblast(s) in the presence of ATRA for a sufficient period of time (e.g., at least two days). Optionally, the contacting step is for 2, 3, 4, 5, 6, 7, 8, 9, 10, 11, 12, 13, 14, 15, or 16 days.

The fibroblast can be genetically modified in vitro or in vivo. The genetic modification can occur before ATRA treatment or concurrently therewith. The fibroblast is genetically modified to overexpress myocardin, for example, by introducing into the fibroblast a heterologous nucleic acid that encodes myocardin. As used herein, introducing in the context of introducing a heterologous nucleic acid into a cell refers to the translocation of the heterologous nucleic acid sequence from outside a cell to inside the cell. In some cases, introducing refers to translocation of the heterologous nucleic acid from outside the cell to inside the nucleus of the cell.

Various methods of translocation are contemplated, including, but not limited to viral infection, transfection, transduction, electroporation, nanoparticle delivery, contact with nanowires or nanotubes, receptor mediated internalization, translocation via cell penetrating peptides, liposome mediated translocation, or any method now known or identified in the future for introduction of nucleic acids into prokaryotic or eukaryotic cellular hosts.

In some cases, the method of translocation is viral infection, for example, using a viral vector. Examples of viral vectors include retroviral, lentiviral, adenoviral, and adeno-associated viral (AAV) vectors. In some cases, for example with adenoviral and AAV vectors, the vector is not integrated into the genome of fibroblasts. In other cases, for example with retroviral and lentiviral vectors, the vector may integrate into the genome of fibroblasts.

A targeted nuclease system (e.g., an RNA-guided nuclease, a transcription activator-like effector nuclease (TALEN), a zinc finger nuclease (ZFN), or a megaTAL (MT) (see, for example, Li et al., Signal Transduct. Target. Ther. 5 (1): 1 (2020)) can also be used to introduce a heterologous nucleic acid, for example, a heterologous nucleic acid encoding myocardin, into a fibroblast.

The CRISPR/Cas9 system, an RNA-guided nuclease system that employs a Cas9 endonuclease, can be used to modify genomic DNA in fibroblasts, for example, by inserting into the fibroblast a heterologous nucleic acid sequence encoding myocardin. As used throughout, the CRISPR/Cas system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

As used herein, Cas9 refers to an RNA-mediated nuclease (e.g., of bacterial or archeal origin, or derived therefrom). Exemplary RNA-mediated nucleases include the foregoing Cas9 proteins and homologs thereof. Other RNA-mediated nucleases include Cpf1 (See, e.g., Zetsche et al., Cell 163 (3): 759-771 (2015)) and homologs thereof.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi et al., RNA Bio. 10 (5): 726-737 (2013); Hou et al., Proc. Natl. Acad. Sci. 110 (39): 15644-15649 (2011); and Sampson et al., Nature 497 (7448): 254-257 (2013). Variants of any of the Cas9 nucleases provided herein can be optimized for efficient activity or enhanced stability in the fibroblast. Thus, engineered Cas9 nucleases are also contemplated. In some cases, the engineered Cas9 is engineered such that the endonuclease domain is inactive, i.e., dCas9. See, for example, Chakraborty, et al., Stem Cell Reports, 3 (6): 940-947 (2014); Black, et al., Cell Stem Cell, 19 (3): 406-414 (2016); Rubio, et al., Sci. Rep., 6:37540 (2016); Liu, et al., Cell Stem Cell, 23:758-771 (2018); Wang, et al., Acta. Pharm. Sin. B., 10 (2): 313-326 (2020); and Jiang, et al., Mol. Ther., 30:54-74 (2022).

The heterologous nucleic acid that encodes myocardin may be, for example, SEQ ID NO: 71 or a nucleic acid sequence having at least 85, 90, 95, or 99% identity with the nucleic acid sequence comprising or consisting of SEQ ID NO: 71.

Percentage of sequence identity is determined by comparing two optimally aligned sequences over a comparison window, wherein the portion of the polynucleotide sequence in the comparison window may comprise additions or deletions (i.e., gaps) as compared to the reference sequence (e.g., SEQ ID NO: 71 or SEQ ID NO: 72) which does not comprise additions or deletions, for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

The terms identical or percent identity, in the context of two or more nucleic acids refer to two or more sequences that are the same sequences. Two sequences are substantially identical if two sequences have a specified percentage (e.g., 85%, 90%, 95%, or 99%) of nucleotides that are the same when compared and aligned for maximum correspondence over a comparison window, or designated region as measured using one of the following sequence comparison algorithms or by manual alignment and visual inspection.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the numbers of contiguous positions selected from the group consisting of from 50 to 600, usually about 75 to about 200, more usually about 100 to about 150 in which a sequence may be compared to a reference sequence of the same number of contiguous positions after the two sequences are optimally aligned. Methods of alignment of sequences for comparison are well known in the art.

An algorithm for determining percent sequence identity and sequence similarity is the BLAST 2.0 algorithms, e.g., as described in, Altschul et al., J. Mol. Biol., 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands.

As used throughout, the term nucleic acid or nucleotide refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. A person of skill in the art would recognize that a particular nucleic acid sequence can be modified to encompasses conservatively modified variants thereof (e.g., degenerate codon substitutions), alleles, orthologs, SNPs, and complementary sequences while retaining the function of the reference sequence, in this case the sequence encoding myocardin. Any of the nucleic acid sequences described herein can be codon-optimized.

As used herein, myocardin (SEQ ID NO: 72) is a protein encoded by the MYOCD gene. As used throughout, the terms polypeptide, peptide, and protein are used interchangeably herein to refer to a polymer of amino acid residues. The terms encompass amino acid chains of any length, including full-length proteins, wherein the amino acid residues are linked by covalent peptide bonds.

As used herein, a gene is a segment of DNA involved in producing or encoding a polypeptide chain. It may include regions preceding and following the coding region (e.g., leader and trailer sequences) as well as intervening sequences (e.g., introns) between individual coding segments (exons).

As used throughout, heterologous refers to what is not normally found in nature. For example, a heterologous nucleotide sequence refers to a nucleotide sequence not normally found in a given cell in nature. As such, a heterologous nucleotide sequence may be foreign to its host cell (i.e., is exogenous to the cell); naturally found in the host cell (i.e., endogenous) but present at an unnatural quantity in the cell (i.e., greater or lesser quantity than naturally found in the host cell); or be naturally found in the host cell but positioned outside of its natural locus.

### Reprogrammed Smooth Muscle Cells

Provided herein is a rSMC. Optionally, the rSMC is prepared by any one of the methods described herein; however, the rSMC can be the progeny of a rSMC that is genetically modified to express or overexpress a heterologous myocardin encoding nucleic acid and treated with ATRA. The rSMC comprises a heterologous nucleic acid sequence encoding myocardin. Optionally, the rSMC cell co-expresses CNN1 and SMTN in a non-striated pattern. Smooth muscles cells lack sarcomeres, and as such do not have striations like cardiac and skeletal muscle cells. Smooth muscle cells contract using actin and myosin filaments interaction in the presence of intracellular calcium.

rSMCs also comprise and express one or more genes (e.g., CNN1, Calponin, and SMTN) present in naturally occurring smooth muscle cells, As used herein, CNN1 is a gene encoding Calponin 1, a protein thought to regulate actin filaments in smooth muscle cells. SMTN a gene which encodes Smoothelin, a protein marker for fully differentiated smooth muscle cells.

Optionally, the rSMC provided herein contracts by more than 10% (including, for example, by more than 20% or more than 25%) in the presence of sufficient amount of a vasoactive agent. A vasoactive agent can be selected from the group consisting of carbachol, endothelin-1, or potassium chloride. A sufficient amount of the vasoactive agent is an amount sufficient to release intracellular calcium in the rSMC and is an amount that promotes contraction of naturally occurring smooth muscle cells. Contraction can be measured by, for example, change in cell surface area by more than 10%, including, for example, at least 11, 12, 15, 20, or 25%. Notably, vasoactive agents reduce the surface area of an untransduced HDF by less than 10%. The contraction seen in the rSMCs in response to a vasoactive agent is greater than that seen in an untransduced HDF.

### Compositions Comprising Reprogrammed Smooth Muscle Cells

The rSMCs described herein can be formulated as a pharmaceutical composition. Optionally, the pharmaceutical composition can further comprise a pharmaceutically acceptable carrier. As used throughout, a carrier is a compound, composition, substance, or structure that, when in combination with a compound or cells, aids or facilitates preparation, storage, administration, delivery, effectiveness, selectivity, or any other feature of the cells for its intended use or purpose. For example, a carrier can be selected to minimize any degradation of the cells and to minimize any adverse side effects upon introduction of the composition into a subject. Such pharmaceutically acceptable carriers include sterile biocompatible pharmaceutical carriers, including, but not limited to, saline, buffered saline, dextrose, and water. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

The rSMCs can be formulated as a pharmaceutical composition for parenteral administration or for local administration (e.g., intramuscularly) at or near an ischemic site. In some examples, the pharmaceutical composition further comprises a second therapeutic agent, including agents that promote perfusion directly or indirectly, for example, an angiotensin-converting enzyme (ACE) inhibitor, angiotensin II receptor blocker (ARB), anti-platelet agent, nitrate, beta-blocker, calcium-channel blocker, or anti-coagulant.

### Treatment Methods

Also provided is a method of treating a subject with ischemia or at risk of developing ischemia. The method comprises administering to the subject an effective amount of a rSMC as described herein; a population of reprogrammed smooth muscle cells described herein; or a pharmaceutical composition described herein.

As used herein, ischemia refers to a vascular condition in which blood supply to a bodily organ, tissue, or part is decreased. Ischemia may be caused by atherosclerotic occlusion of blood vessels resulting from, for example, peripheral artery disease, coronary artery disease, stroke, or heart attack. Ischemia may be characterized by low blood circulation and eventual tissue necrosis. Ischemia is reduced by production of collateral blood supply and neovascularization that permit refusion of the organ, tissue or body part. Ischemic disease, by way of example, can affect limbs, digits, muscles, heart, liver, brain and the like.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn, and pediatric subjects, whether male or female, are intended to be covered. As used herein, patient or subject may be used interchangeably and can refer to a subject diagnosed with or at risk of developing a disorder. The term patient or subject includes human and veterinary subjects. In any of the methods provided herein, the subject can be a subject diagnosed with ischemia or at risk of developing ischemia.

The fibroblasts from which the rSMCs are derived can be from the same subject to be treated (i.e., for an autologous cell transplant) or can be derived from a different donor (i.e., for an allogeneic cell transplant). The allogeneic cells can optionally be derived from a genetically related donor. Allogenic transplantation may require treatment for immune suppression, which may optionally be discontinued after neovascularization or reperfusion occurs.

As used herein, the terms treatment, treat, or treating refers to a method of reducing one or more of the effects of the disorder or one or more symptoms of the disorder, for example, ischemia in the subject. Thus in the disclosed methods, treatment can refer to a 10%, 20%, 30%, 40%, 50%, 60%, 70%, 80%, 90%, or 100% reduction in the severity of ischemia. For example, a method for treating ischemia is considered to be a treatment if there is a 10% reduction in one or more symptoms of the ischemia in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

As used herein, administer or administration refers to the act of introducing, injecting or otherwise physically delivering a substance as it exists outside the body (e.g., rSMCs or cells differentiated therefrom) into a subject, such as by intracardiac, intravenous, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

In the treatment methods described herein, the cells, population of cells, or pharmaceutical composition is administered in an effective amount. As used herein, the term effective amount or therapeutically effective amount refers to an amount of a composition comprising any of the rSMCs described herein, or cells differentiated therefrom, that, when administered to a subject, is effective, alone or in combination with additional agents, to treat a disease or disorder either by one dose or over the course of multiple doses. A suitable dose can depend on a variety of factors including the particular rSMCs or cell differentiated therefrom used and whether they are used concomitantly with other therapeutic agents. Other factors affecting the dose administered to the subject include, e.g., the type or severity of the disease.

Exemplary amounts of effective amounts of rSMCs or cells differentiated therefrom can be determined by one of ordinary skill in the art. Factors that influence dosage can include, e.g., other medical disorders concurrently or previously affecting the subject, the general health of the subject, the genetic disposition of the subject, diet, time of administration, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

Optionally, the effective amount increases vascular perfusion, neovascularization, and/or arteriogenesis (i.e., formation or larger vessels such as arteries and arterioles) in the subject. The effect can be measured by, for example, by laser Doppler perfusion imaging.

### Additional Definitions

As used in this specification and the appended claims, the singular forms “a,” “an,” and “the” include plural reference unless the context clearly dictates otherwise.

The use of any and all examples or exemplary language (e.g., “for example” or “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

### EXAMPLES

The following examples are set forth below to illustrate the compositions, methods, and results according to the disclosed subject matter. These examples are not intended to be inclusive of all aspects of the subject matter disclosed herein but rather to illustrate representative methods, compositions, and results. These examples are not intended to exclude equivalents and variations of the present disclosure, which are apparent to one skilled in the art.

### Example 1: Generation of Smooth Muscle-Like Cells by a Direct Reprogramming Approach

### Materials and Methods

Cell culture and maintenance. Human dermal fibroblasts (HDFs) were isolated from the dermis of human juvenile foreskin and expanded in Dulbecco's Modified Eagle's medium (DMEM) (Lonza, Basel, SWI) supplemented with 10% fetal bovine serum (FBS) (Sigma-Aldrich, St. Louis, MO), 1× Antibiotic-Antimycotic (Anti-Anti) (Gibco, Waltham, MA), 1× MEM Non-Essential Amino Acids (MEM NEAA) (Gibco, Waltham, MA), and 1× GlutaMAX Supplement (GlutaMAX) (Gibco, Waltham, MA) at 37° C. with 5% CO₂. The Platinum-A (Plat-A) retroviral packaging cell line (Cell Biolabs, San Diego, CA) was maintained in the same medium without Anti-Anti and used for transfection at passages four to seven. HDFs and directly reprogrammed smooth muscle cells (rSMCs) were maintained in DMEM with low glucose (HyClone, Logan, UT) supplemented with 5% FBS, Anti-Anti, MEM NEAA, and GlutaMAX.

Generation of retroviruses. Retroviral construct was generated by subcloning human MYOCD complementary deoxyribonucleic acid (cDNA) into a retroviral vector, pMXs. The construct was transfected into Plat-A cells using FuGENE HD (Promega, Madison, WI), according to the manufacturer's instructions. The viral supernatant was collected at days 2-, 4-, and 6-days post-transfection and filtered through a 0.45 μm polyethersulfone (PES) membrane filter (Corning, Corning, NY). Titration of retroviruses was performed using Retro-X qRT-PCR Titration Kit (Takara Bio, Shiga, JP), according to the manufacturer's instructions.

Generation of directly reprogrammed SMCs. For direct reprogramming of HDFs into contractile SMCs, HDFs were seeded at a density of 1.5×10⁵ cells per ml in DMEM (Lonza) supplemented with 10% FBS (Sigma-Aldrich), Anti-Anti (Gibco), MEM NEAA (Gibco), and GlutaMAX (Gibco) at 37° C. with 5% CO₂. The cells were infected overnight with filtered retroviral medium containing four μg per ml of polybrene (Sigma-Aldrich) with or without 0.4, 2, or 10 μM per liter of all-trans retinoic acid (ATRA) (Sigma-Aldrich), and the viral medium was replaced with DMEM/low glucose (HyClone) supplemented with 5% FBS, Anti-Anti, MEM NEAA, and GlutaMAX with or without ATRA for 24 hrs. The viral infection was repeated twice, and the cells were maintained in DMEM/low glucose containing 5% FBS with or without ATRA for the duration of culture.

Qualitative reverse transcriptase polymerase chain reaction (qRT-PCR). Total ribonucleic acid (RNA) was isolated from cells using the RNeasy Mini Kit (QIAGEN, Hilden, GE) or TRIsure (Bioline, Memphis, TN), according to the manufacturer's instructions. The extracted RNA was reverse transcribed with TaqMan Reverse Transcription Reagents (Applied Biosystems, Foster City, CA), according to the manufacturer's instructions. The synthesized cDNA was amplified with PowerUP SYBR Green Master Mix (Thermo Fisher Scientific, Waltham, MA), according to the manufacturer's instructions, and was subject to qRT-PCR with human or mouse specific primers, as shown in Table 1. Quantitative assessment of RNA levels was performed using a QuantStudio 3 96-well 0.2-ml real-time PCR system (Applied Biosystems). The relative messenger RNA (mRNA) expression was normalized to GAPDH.

TABLE 1
Primers used for qRT-PCR analysis in Example 1

| Gene | Forward | Reverse |
|---|---|---|
| GAPDII | GGTGGTCTCCTCTGACTTCAACA | GTGGTCGTTGAGGGCAATG |
| | (SEQ ID NO: 1) | (SEQ ID NO: 2) |
| STRA6 | AGACCAGGTCCCACACTGA | TTCATAATAGCCAAAGGCATAA |
| | (SEQ ID NO: 3) | A (SEQ ID NO: 4) |
| MYOCD | ACTCCTGGGGTCTGAGCATT | CTGGACGTTTCAGTGGTGGT |
| | (SEQ ID NO: 5) | (SEQ ID NO: 6) |
| Endogenous | TTACCTCTCGTGGCTTTGGC | CCAGCAAGCTTTGTCAAGACC |
| MYOCD | (SEQ ID NO: 7) | (SEQ ID NO: 8) |
| ACTA2 | AAAAGACAGCTACGTGGGTGA | GCCATGTTCTATCGGGTACTTC |
| | (SEQ ID NO: 9) | (SEQ ID NO: 10) |
| TAGLN | AGTGCAGTCCAAAATCGAGAAG | CTTGCTCAGAATCACGCCAT |
| | (SEQ ID NO: 11) | (SEQ ID NO: 12) |
| CALD1 | CTGTTCCTGCTGAAGGTGTACG | CCTACCTTCAAGCCAGCAGTTTC |
| | (SEQ ID NO: 13) | (SEQ ID NO: 14) |
| DES | GAAATCCGGCACCTCAAGGA | GATGGGGAGATTGATCCGGC |
| | (SEQ ID NO: 15) | (SEQ ID NO: 16) |
| CNN1 | GTCAACCCAAAATTGGCACCA | ACCTTGTTTCCTTTCGTCTTCG |
| | (SEQ ID NO: 17) | (SEQ ID NO: 18) |
| MYH11 | AGAAGCCAGGGAGAAGGAAACCA | TGGAGCTGACCAGGTCTTCCAT |
| | A (SEQ ID NO: 19) | TT |
| | | (SEQ ID NO: 20) |
| SMTN | CCTTCTCATCTGCGGAGACC | CAGTCCACCAGCATCTTCCA |
| | (SEQ ID NO: 21) | (SEQ ID NO: 22) |
| Meta- | CCGCTGAGGTGGGTATAGGT | CTTCCCGATGCAAGGACTGA |
| VCL | (SEQ ID NO: 23) | (SEQ ID NO: 24) |
| TPM1 | GGCACCGAAGATGAACTGGAC | GCGTCTGTTCAGAGAAGCTACG |
| | (SEQ ID NO: 25) | (SEQ ID NO: 26) |
| TPM2 | GAGAGGTCTGTGGCAAAGTTGG | GGAGGTGATGTCATTGAGTGCG |
| | (SEQ ID NO: 27) | (SEQ ID NO: 28) |
| S100A4 | GTGCTTCTGAGATGTGGGCTT | GGGTCAGCAGCTCCTTTAGT |
| | (SEQ ID NO: 29) | (SEQ ID NO: 30) |
| THY1 | CTGAGCACACATACCGCTCC | GCCCTCACACTTGACCAGTT |
| | (SEQ ID NO: 31) | (SEQ ID NO: 32) |
| VIM | GTCCGCACATTCGAGCAAAG | CGCTGCTAGTTCTCAGTGCT |
| | (SEQ ID NO: 33) | (SEQ ID NO: 34) |
| PDGFRA | TTTTTGTGACGGTCTTGGAAGT | TGTCTGAGTGTGGTTGTAATAG |
| | (SEQ ID NO: 35) | C |
| | | (SEQ ID NO: 36) |
| MYH10 | TGGTTTTGAGGCAGCTAGTATCA | AGTCCTGAATAGTAGCGATCCT |
| | (SEQ ID NO: 37) | T |
| | | (SEQ ID NO: 38) |
| SPP1 | CGAGGTGATAGTGTGGTTTATGG | GCACCATTCAACTCCTCGCTTTC |
| | (SEQ ID NO: 39) | (SEQ ID NO: 40) |
| MMP2 | AGCGAGTGGATGCCGCCTTTAA | CATTCCAGGCATCTGCGATGAG |
| | (SEQ ID NO: 41) | (SEQ ID NO: 42) |
| CSPG4 | GTCCTGCCTGTCAATGACCAAC | CGATGGTGTAGACCAGATCCTC |
| | (SEQ ID NO: 43) | (SEQ ID NO: 44) |
| KLF4 | GGGCCCAATTACCCATCCTT | GAAGGTGGGGTGAGCATCAT |
| | (SEQ ID NO: 45) | (SEQ ID NO: 46) |
| SRF | CGAGATGGAGATCGGTATGGT | GGGTCTTCTTACCCGGCTTG |
| | (SEQ ID NO: 47) | (SEQ ID NO: 48) |
| MRTFA | CAAACGGAAGATTCGTTCCCG | TTGAGGTCATCGGCTAGTCTG |
| | (SEQ ID NO: 49) | (SEQ ID NO: 50) |
| MRTFB | CAGATCGTTCTGAACTTGTCAGG | CCCAACGCCTATAATTGCTTCTT |
| | (SEQ ID NO: 51) | (SEQ ID NO: 52) |
| CRIP1 | CCTGCCTGAAGTGCGAGAAAT | CCTTTAGGCCCAAACATGGC |
| | (SEQ ID NO: 53) | (SEQ ID NO: 54) |
| PITX2 | CGGCAGCGGACTCACTTTA | GTTGGTCCACACAGCGATTT |
| | (SEQ ID NO: 55) | (SEQ ID NO: 56) |
| COL1A1 | GAGGGCCAAGACGAAGACATC | CAGATCACGTCATCGCACAAC |
| | (SEQ ID NO: 57) | (SEQ ID NO: 58) |
| COL1A2 | GGCCCTCAAGGTTTCCAAGG | CACCCTGTGGTCCAACAACTC |
| | (SEQ ID NO: 59) | (SEQ ID NO: 60) |
| COL5A1 | GCCCGGATGTCGCTTACAG | AAATGCAGACGCAGGGTACAG |
| | (SEQ ID NO: 61) | (SEQ ID NO: 62) |
| LUM | GGATTGGTAAACCTGACCTTCAT | GATAAACGCAGATACTGCAATG |
| | (SEQ ID NO: 63) | C |
| | | (SEQ ID NO: 64) |
| LOXL1 | GGCTGCTATGACACCTACAATG | GTAGTGAATGTTGCATCTCACC |
| | (SEQ ID NO: 65) | A |
| | | (SEQ ID NO: 66) |
| TNNI1 | GCGATACGACATTGAGGCCAA | TTCCCACGGAGGTCCATCA |
| | (SEQ ID NO: 67) | (SEQ ID NO: 68) |
| TNNI3 | TTTGACCTTCGAGGCAAGTTT | TGCAGAGATCCTCACTCTCCG |
| | (SEQ ID NO: 69) | (SEQ ID NO: 70) |

Flow cytometry. Cells were washed with Dulbecco's Phosphate-Buffered Saline (DPBS; Corning), detached with ACCUTASE (STEMCELL Technologies, Vancouver, BC), and harvested with autoMACS Running Buffer (Miltenyi Biotec, Bergisch Gladbach, GE). The cells were fixed and permeabilized with Cytofix/Cytoperm Fixation/Permeabilization Kit (BD Biosciences, Franklin Lakes, NJ), according to the manufacturer's instructions. The cells were then incubated with non-conjugated primary antibodies overnight at 4° C. in the dark, followed by fluorochrome-labelled secondary antibodies for at least three hours, according to the manufacturer's instructions. Primary and secondary antibodies are shown in Table 2. The fluorescence-activated cells were analyzed by a LSRFortessa Flow Cytometer (BD Biosciences). Flow cytometric data were analyzed with FlowJo™ v10.8 Software (BD Biosciences) using appropriate isotype-matched controls.

Immunocytochemistry. Cells were washed with DPBS (Corning), fixed in 4% paraformaldehyde (VWR, Radnor, PA) in the dark for half an hour at room temperature, and permeabilized with 0.5-1% Triton X-100 (Sigma-Aldrich) in DPBS in the dark for an hour at RT. The permeabilized cells were then incubated with blocking buffer containing 0.5-1% Triton X-100 and 1% bovine serum albumin (Miltenyi Biotec) in DPBS in the dark for three hours at RT. The cells were incubated with non-conjugated primary antibodies overnight at 4° C. in the dark, followed by fluorochrome-labelled secondary antibodies for three hours at 4° C. in the dark, according to the manufacturer's instructions. Primary and secondary antibodies are shown in Table 2. The cells were counter-stained with DAPI (Invitrogen, Carlsbad, CA) to visualize the nuclei. The images were captured by a Zeiss LSM 700 or 780 confocal microscope (Carl Zeiss AG, Oberkochen, GE).

TABLE 2
Antibodies

| | Vendor or | | Working |
|---|---|---|---|
| Target antigen | Source | Catalog # | concentration |
| Recombinant Anti- alpha | Abcam | ab32575 | 1:100 |
| smooth muscle Actin | | | |
| antibody [E184] | | | |
| Anti-Actin, smooth muscle | Sigma-Aldrich | CBL171-I | 1:100 |
| Antibody, clone | | | |
| ASM-1/1A4 | | | |
| Anti- TAGLN/Transgelin | Abcam | ab10135 | 1:100 |
| antibody | | | |
| Anti- TAGLN/Transgelin | Abcam | ab14106 | 1:100 |
| antibody | | | |
| Calponin 1 Monoclonal | Invitrogen | MA5-11620 | 1:100 |
| Antibody (CALP) | | | |
| Smoothelin (H- 300) | Santa Cruz | sc-28562 | 1:100 |
| | Biotechnology | | |
| Anti-smooth muscle Myosin | Abcam | ab82541 | 1:100 |
| heavy chain 11 antibody | | | |
| MYH11 (G-4) | Santa Cruz | sc-6956 | 1:100 |
| | Biotechnology | | |
| Goat anti-Mouse IgG | Invitrogen | A28175 | 1:500-2000 |
| (H + L), Superclonal | | | |
| Recombinant Secondary | | | |
| Antibody, Alexa Fluor 488 | | | |
| Goat anti-Mouse IgG | Invitrogen | A28180 | 1:500-2000 |
| (H + L), Superclonal | | | |
| Recombinant Secondary | | | |
| Antibody, Alexa Fluor 555 | | | |
| Goat anti-Mouse IgG | Invitrogen | A21235 | 1:500-2000 |
| (H + L), Superclonal | | | |
| Recombinant Secondary | | | |
| Antibody, Alexa Fluor 647 | | | |
| Goat anti-Rabbit IgG | Invitrogen | A27034 | 1:500-2000 |
| (H + L), Superclonal | | | |
| Recombinant Secondary | | | |
| Antibody, Alexa Fluor 488 | | | |
| Goat anti-Rabbit IgG | Invitrogen | A27039 | 1:500-2000 |
| (H + L), Superclonal | | | |
| Recombinant Secondary | | | |
| Antibody, Alexa Fluor 555 | | | |
| Goat anti-Rabbit IgG | Invitrogen | A27040 | 1:500-2000 |
| (H + L), Superclonal | | | |
| Recombinant Secondary | | | |
| Antibody, Alexa Fluor 647 | | | |
| Donkey anti-Goat IgG | Invitrogen | A11055 | 1:500-2000 |
| (H + L), Superclonal | | | |
| Recombinant, Secondary | | | |
| Antibody, Alexa Fluor 488 | | | |

### Results

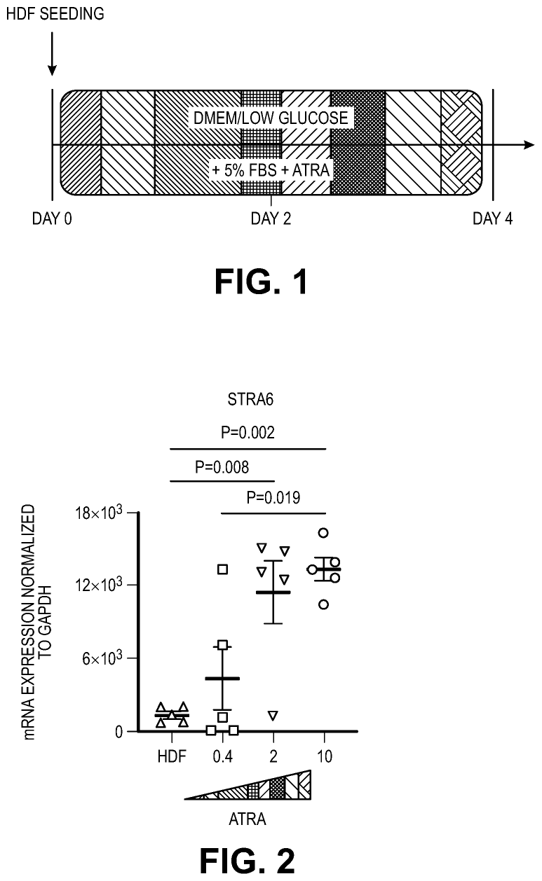

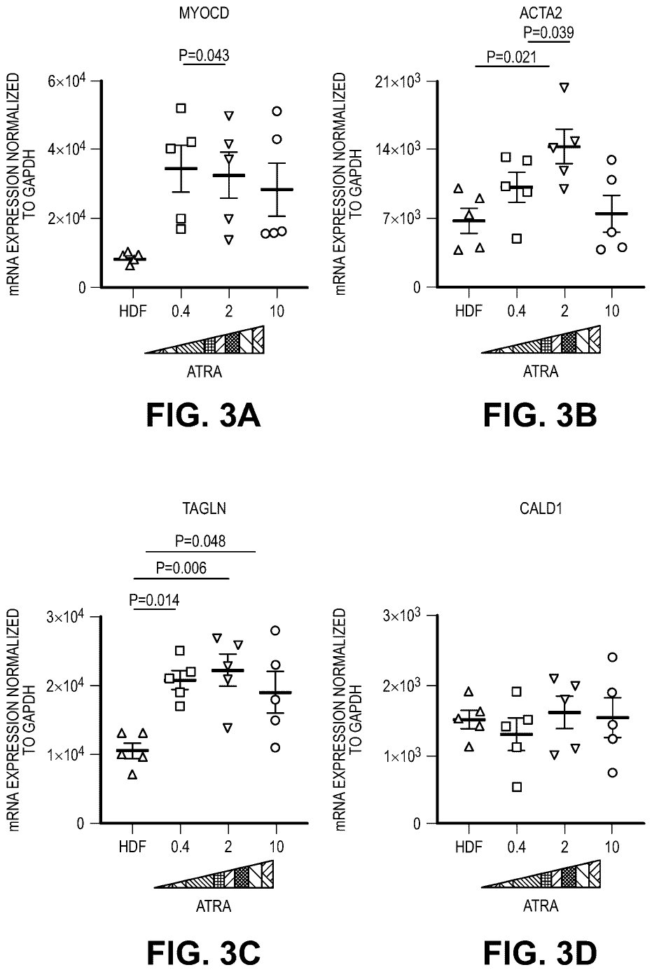

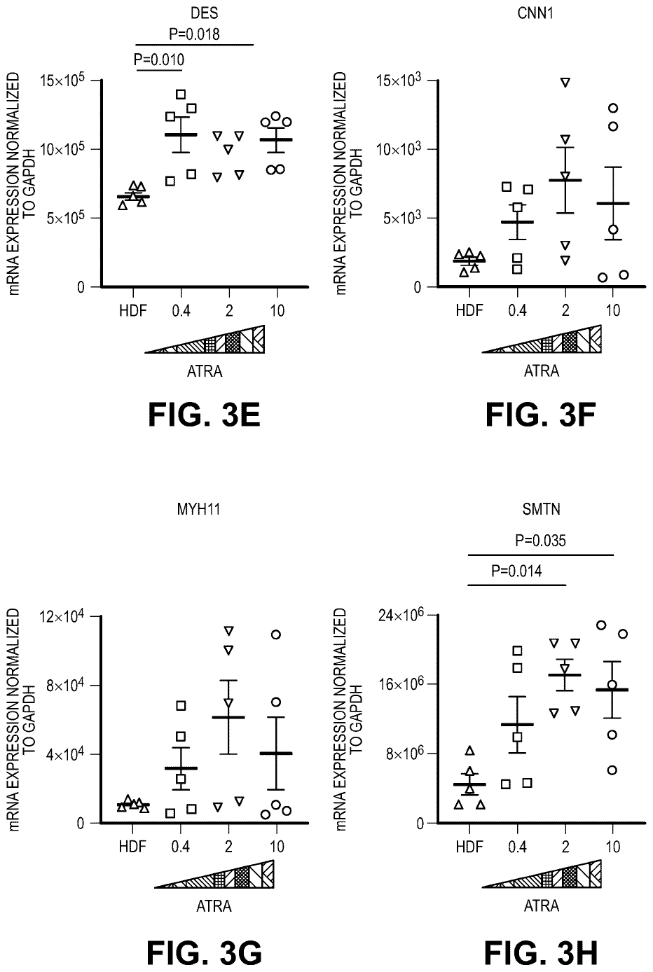

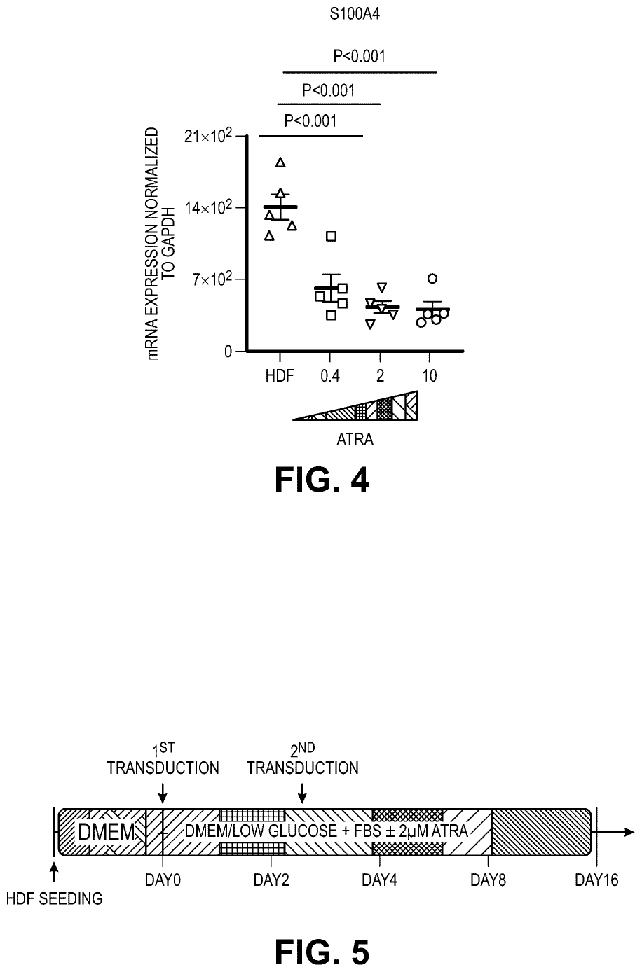

ATRA alone can only marginally induce SMC genes in HDFs. As shown in FIG. 1, HDFs were treated with various concentrations of ATRA for four days. mRNA expression of STRA6, S100A4, and the SMC genes (MYOCD, ACTA2, TAGLN, CALD1, DES, CNN1, MYH11, and SMTN) in ATRA-treated and untreated HDFs was examined by qRT-PCR. As shown in FIGS. 2-3, mRNA expression of STRA6 and the SMC genes was increased in ATRA-treated HDFs compared to the untreated HDFs. The expression of S100A4 was substantially reduced (FIG. 4). Among the tested ATRA concentrations, 2 μM was the most effective for induction of SMC gene expression (FIG. 3). Overall, the fold increase of SMC gene expression ranged from 1.1 (CALD1) to 6.1 (MYH11) (FIG. 3). These results imply that ATRA alone can only marginally induce SMC genes in HDFs.

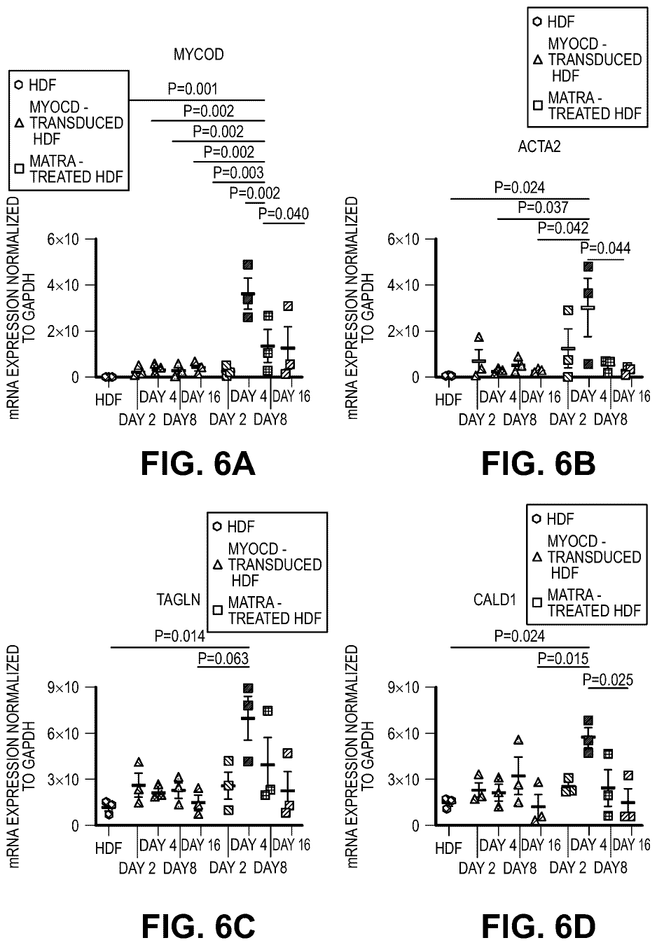

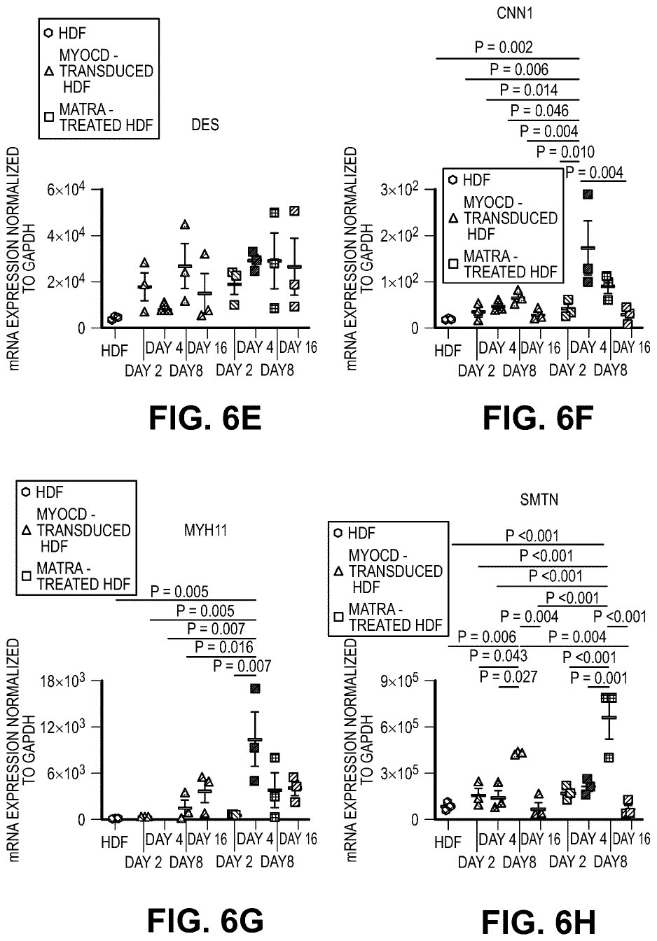

Overexpression of MYOCD combined with ATRA induces robust SMC gene expression in HDFs. As shown in FIG. 5, HDFs were infected twice with retroviral-MYOCD alone or in combination with 2 μM of ATRA and cultured for 16 days. qRT-PCR showed that MYOCD-transduced HDFs expressed the eight SMC genes as early as day 2 but mostly peaked at day 8 (FIG. 6). The peak expression of MYOCD was increased to 295.4-fold, whereas the expression of other SMC genes was increased between 2.2-(TAGLN, CALD1) to 29.2- (MYH11) fold (FIG. 6). Notably, the ATRA and MYOCD (MATRA)-treated HDFs (also referred to herein as reprogrammed SMCS or rSMCs) showed a much more robust expression of the eight SMC genes, which peaked at day 4 except for SMTN, which peaked at day 8 (FIG. 6). As shown in FIG. 6, the fold increase of SMC gene expression ranged from 3.9 (CALD1) to 2639.2 (MYOCD). While gradually decreased over time, these contractile SMC genes were not silenced and mostly remained higher for 16 days (FIG. 6).

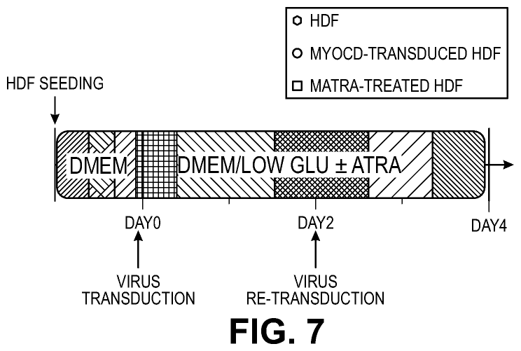

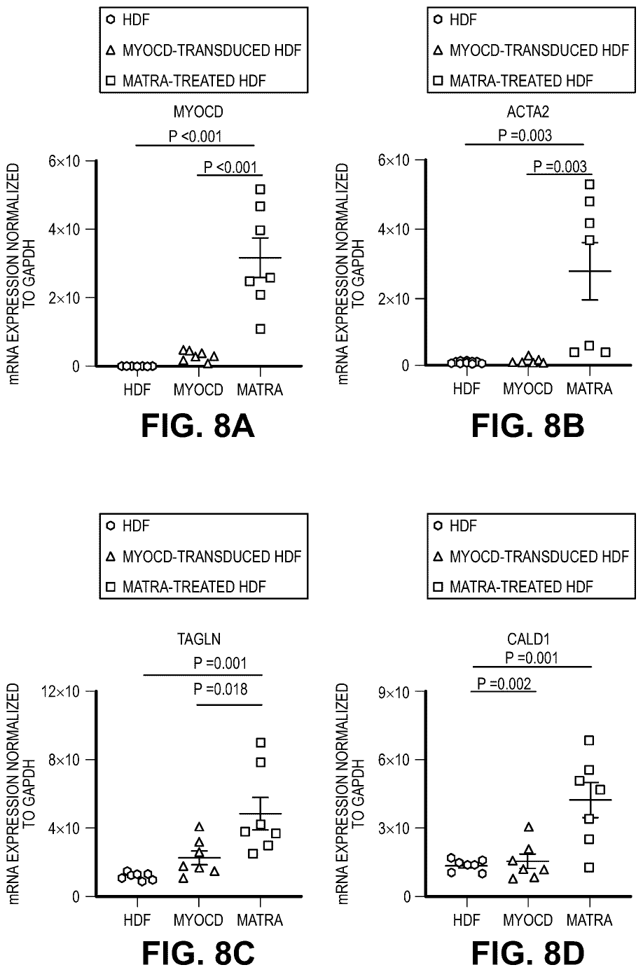

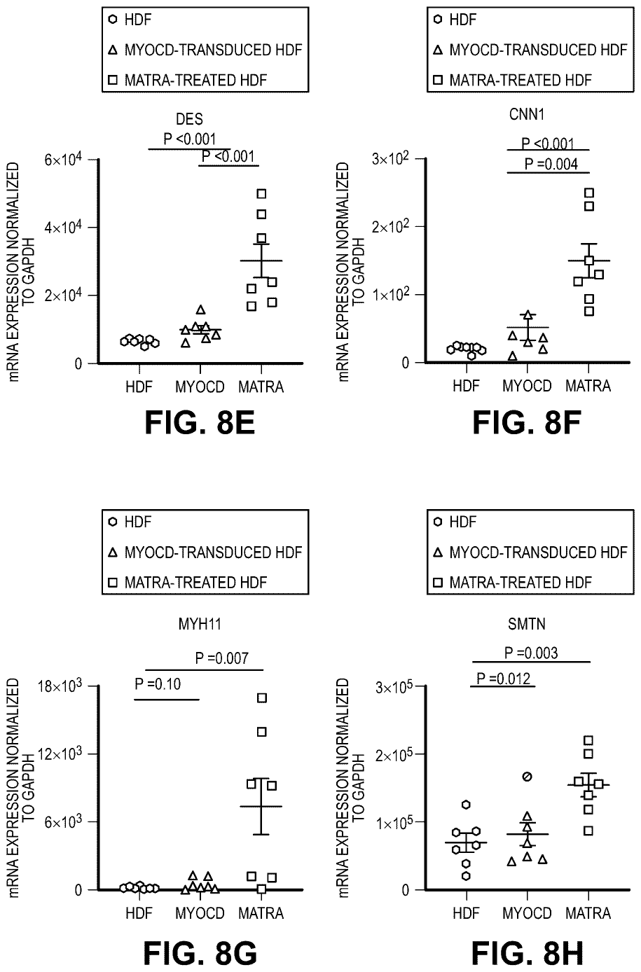

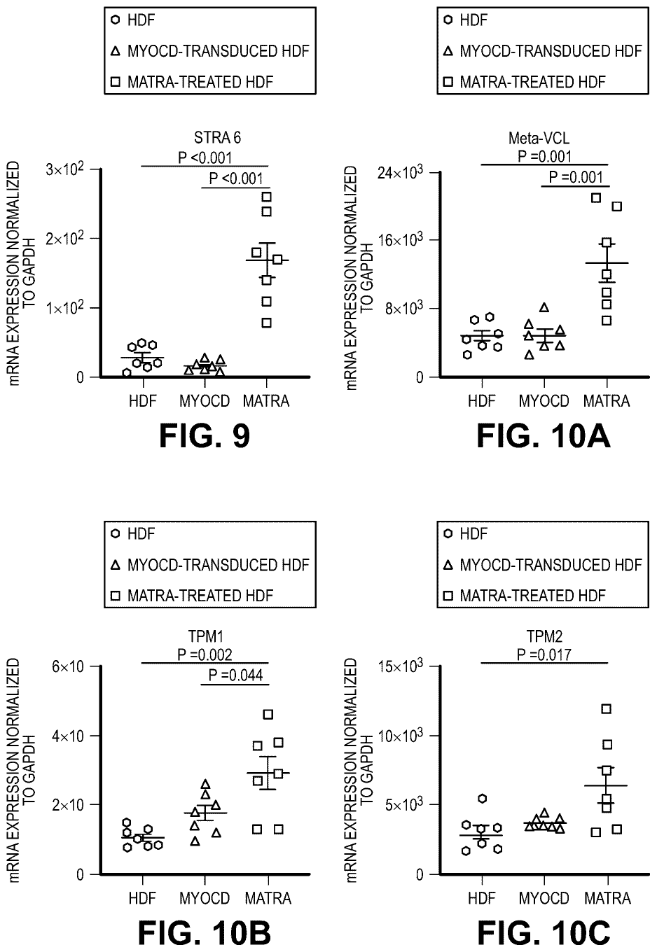

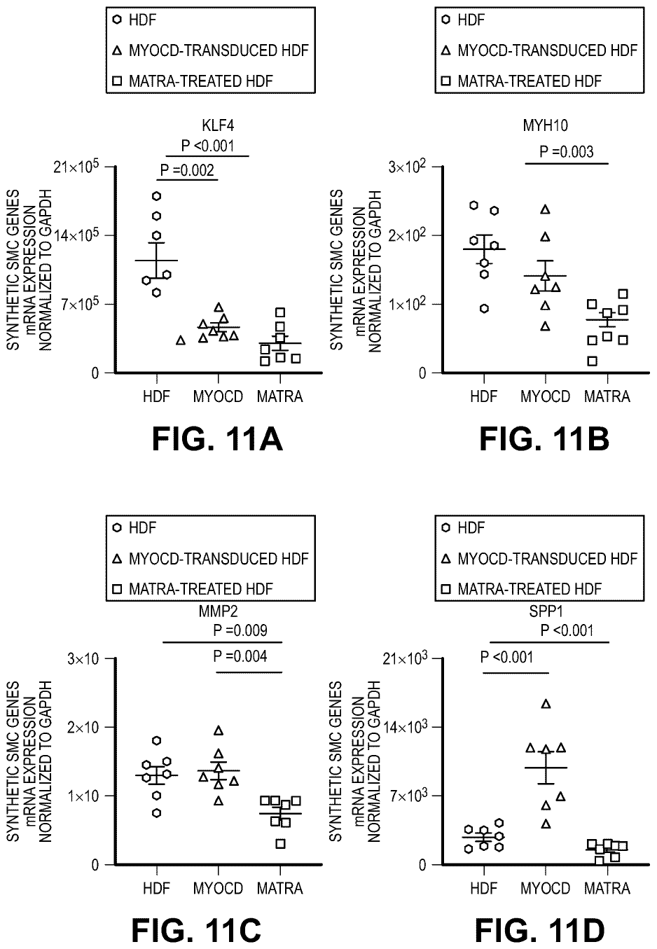

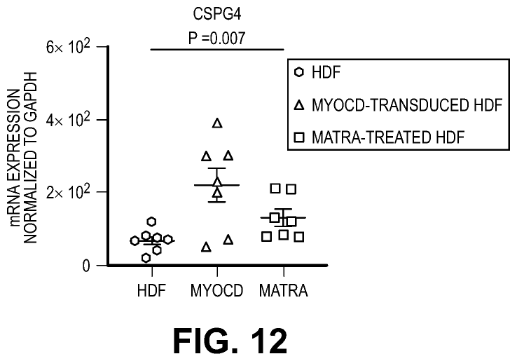

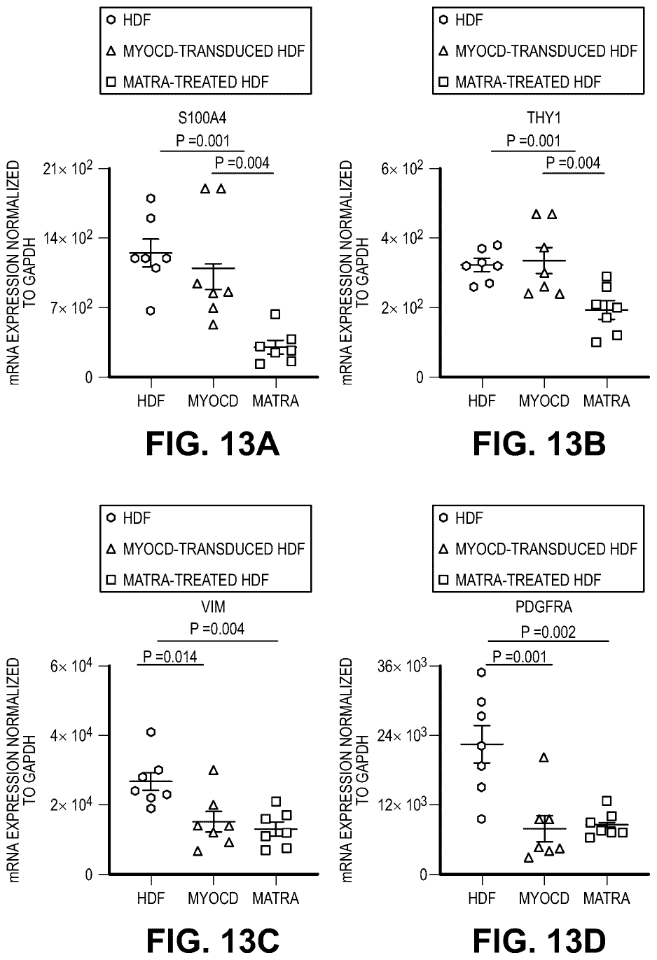

MYOCD and ATRA can increase contractile SMC and pericyte gene expression in HDFs. Expression of the SMC genes in MATRA-treated HDFs mostly peaked at day 4 (FIG. 6). MYOCD, in particular, gradually decreased over time but remained higher in MATRA-treated HDFs than in untransduced HDFs and MYOCD-transduced HDFs (FIG. 6). As shown in FIG. 7, day 4 was chosen as a target date for characterizing the reprogrammed SMCs. qRT-PCR demonstrated that the expression of MYOCD was significantly increased in MATRA-treated HDFs, which was 2,642.9-fold higher compared to the untransduced HDFs and 10.0-fold higher to the MYOCD-transduced HDFs (FIG. 8). The other SMC genes and STRA6 were also markedly increased in MATRA-treated HDFs compared to untransduced HDFs: 34.7- (ACTA2), 4.0- (TAGLN), 3.0- (CALD1), 4.6- (DES), 7.6- (CNN1), 51.4- (MYH11), 2.3- (SMTN), and 5.0- (STRA6) fold (FIGS. 8-9). Interestingly, only in MATRA-treated HDFs, the expression of three SMC genes (VCL, TPM1, and TPM2) that play an important role in the regulation of SMC contraction was significantly increased (FIG. 10). On the other hand, four representative genes specific to synthetic SMCs (KLF4, MYH10, MMP 2, and SPP1) were reduced in MATRA-treated HDFs (FIG. 11). In addition, expression of CSPG4, a representative pericyte gene, was increased in both MYOCD-transduced and MATRA-treated HDFs, compared to untransduced HDFs because a continuous phenotypic transition from pericytes to vascular SMCs and vice versa exists (FIG. 12). Lastly, the mRNA expression of four representative fibroblast genes, such as S100A4, THY1, VIM and PDGFRA, was substantially decreased in this reprogramming condition (FIG. 13). Together, the results indicate that overexpression of MYOCD together with ATRA treatment can substantially increase contractile SMC and pericyte genes in HDFs.

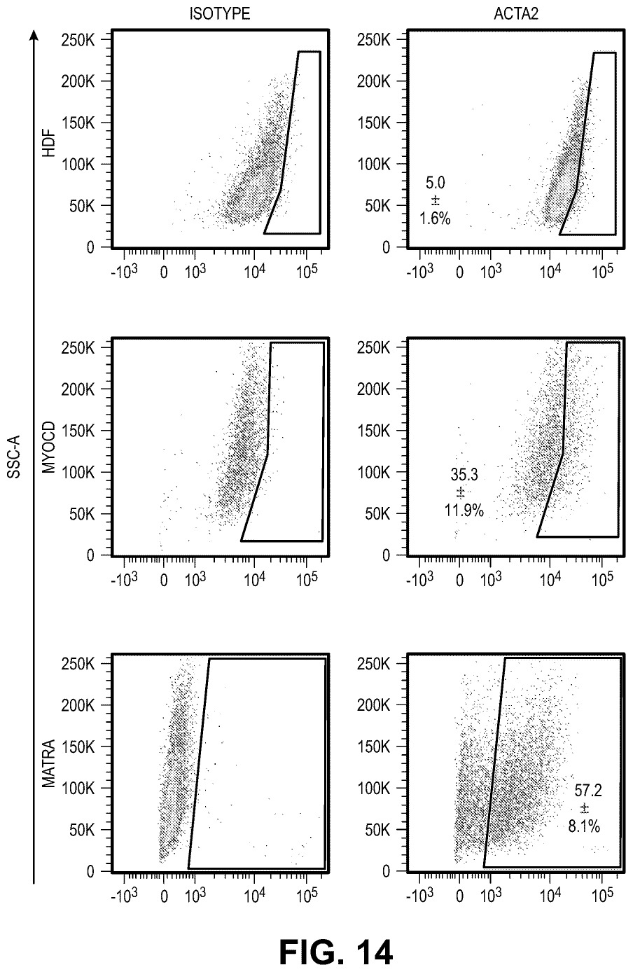

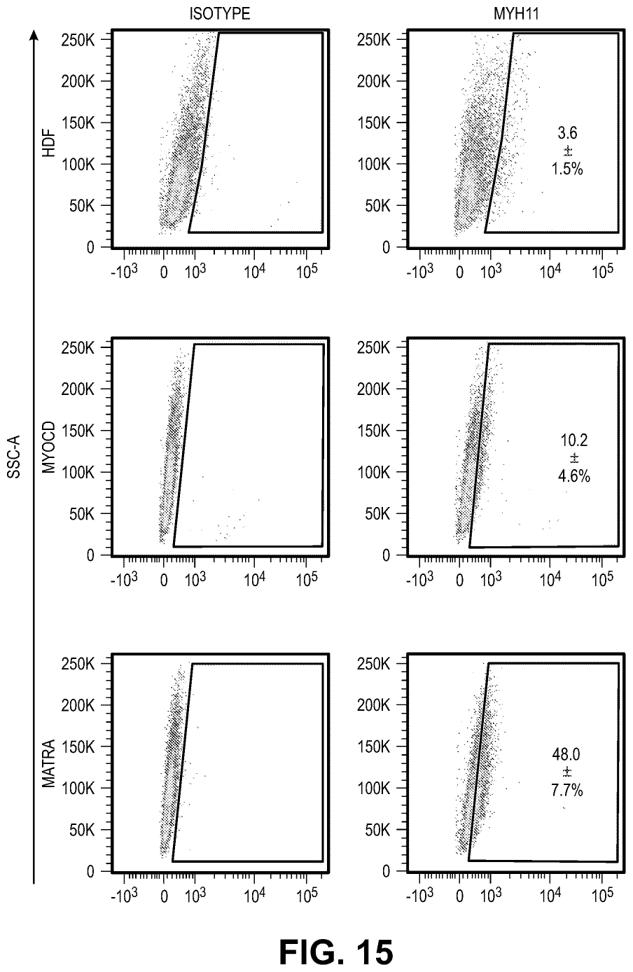

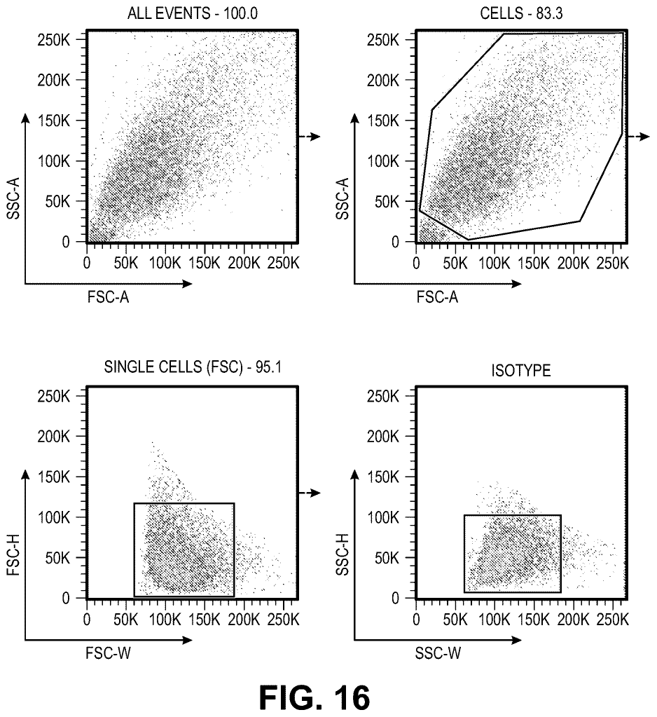

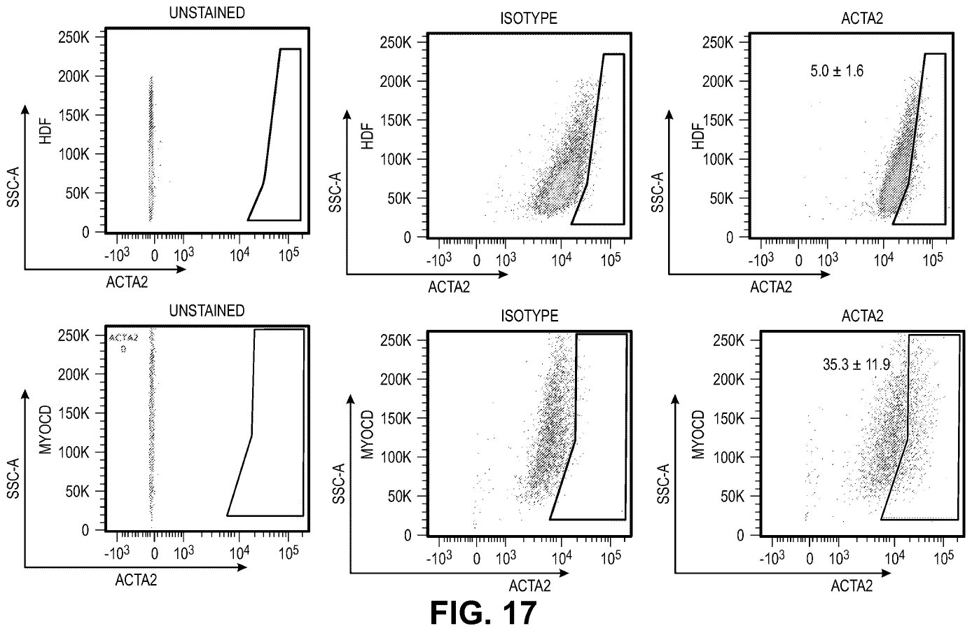

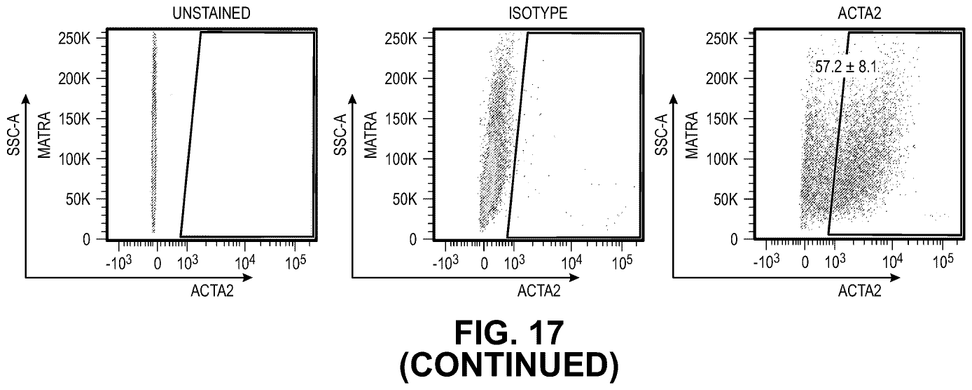

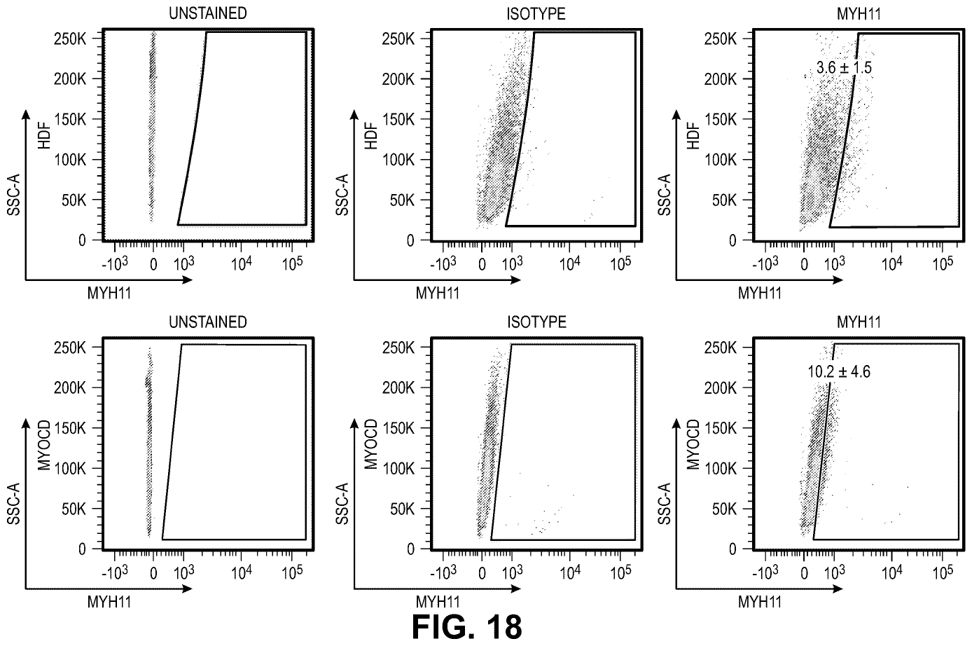

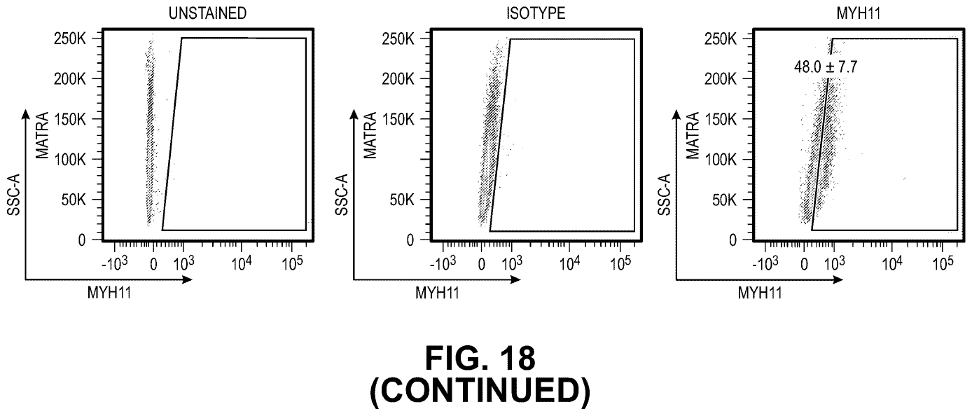

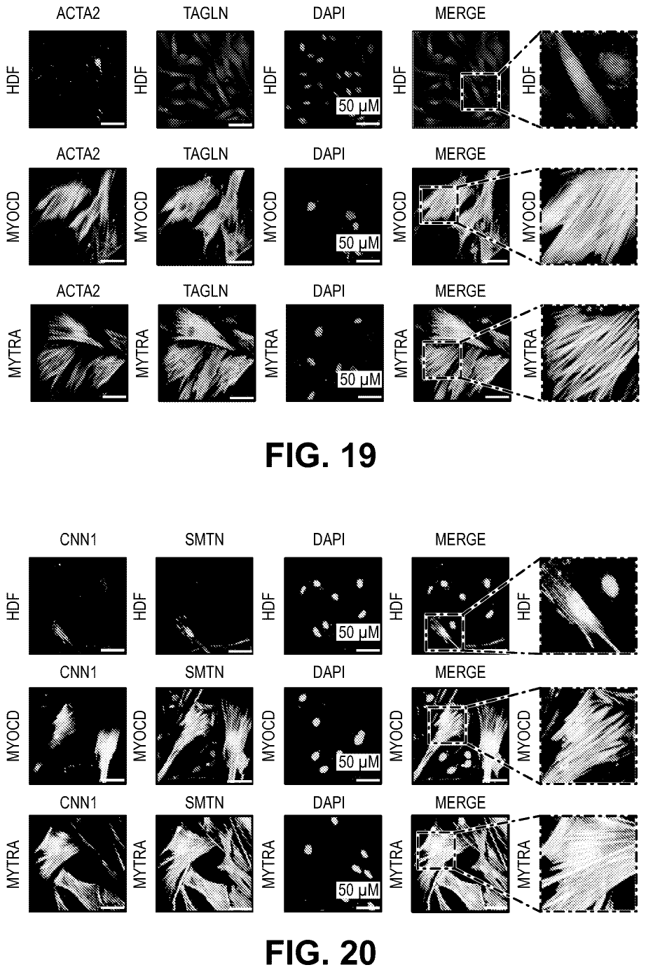

Protein analysis confirms the combination of ATRA and MYOCD can drive a stronger change towards a SMC phenotype in HDFs. Expression of SMC genes was confirmed at the protein level. Flow cytometric analyses showed that MATRA-treated HDFs exhibited ACTA2 and MYH11 at day 4 at ~57% and ~48% of the cells, respectively (FIGS. 14-15). The gating strategy for flow cytometry is shown in FIGS. 16-18. FIG. 16 shows gating used for selection of single live cells. FIGS. 17-18 show gating used for identification of ACTA2+ and MYH11+ cells, respectively. As shown in FIGS. 19-20, immunocytochemistry further confirmed expression of four SMC markers, ACTA2, TAGLN, CNN1, and SMTN. In the untransduced HDFs, ACTA2, CNN1, and SMTN were barely detected with diffuse staining of TAGLN. In MYOCD-transduced HDFs, ACTA2 and TAGLN were clearly expressed, whereas CNN1 and SMTN were expressed with limited actin cytoskeletal organization. MATRA-treated HDFs robustly expressed ACTA2 and TAGLN and possessed a much more prominent actin stress pattern. MATRA-treated HDFs co-expressed CNN1 and SMTN in a typical non-striated SMC-like pattern. While overexpression of MYOCD is indispensable for direct reprogramming of HDFs into SMCs, addition of ATRA showed synergistic effects on the induction of contractile SMC genes. Collectively, these data suggest that a combination of MYOCD and ATRA can drive a stronger cell-fate change towards a contractile SMC phenotype.

### Example 2: Contractile Features of rSMCs

### Materials and Methods

Contractility assessment. HDFs were cultured and directly reprogrammed to SMCs in the same way as described above. Cells were treated with 100 μM carbachol (Sigma-Aldrich) for approximately 10 minutes. Cells were seeded onto a glass-bottom dish (Thermo Fisher Scientific). Contraction was monitored at the cellular level and acquired as time-series at the rate of one frame every 30 seconds over 15 minutes using a Zeiss LSM 700 or 780 confocal microscope (Carl Zeiss AG), which was equipped with a Zeiss stage-top microscope incubator system. Change in cell surface area before and after addition of carbachol was assessed by a modular image acquisition, processing, and analysis software, ZEISS Efficient Navigation (blue edition) (ZEN (blue edition); Carl Zeiss AG).

Collagen gel contraction assay. Cells were washed with DPBS (Corning), detached with 0.25% Trypsin-EDTA (Gibco), and resuspended in 0.4 ml of the medium at a density of 1.5×10⁵ cells. The collagen lattice was prepared by mixing the cell suspension and 0.2 ml of rat tail collagen type I (3 mg per ml; Gibco) and quickly transferred into a 4-well plate after adding the appropriate volume of 1 mole per liter of sodium chloride (Sigma-Aldrich). The collagen gel was polymerized at room temperature for 20 minutes, dissociated from the well, and incubated at 37° C. with 5% CO₂ for two days. Change in diameter of gel was captured by a digital camera and assessed by ImageJ (U.S. National Institutes of Health).

Detection of calcium release. Cells were washed with DPBS (Corning) and preloaded with the calcium-sensitive fluorescent dye, Fluo-4, AM (Thermo Fisher Scientific), in Opti-MEM with reduced serum medium (Gibco) at 37° C. with 5% CO₂ for an hour. The preloaded cells were then washed at 37° C. with 5% CO₂ for 15 minutes for de-esterification of intracellular acetoxymethyl esters. Changes in the intracellular calcium release were acquired as time-series at the rates of one frame every 0.2 milliseconds over 550 seconds using a Zeiss LSM 700 or 780 confocal microscope (Carl Zeiss AG) before and after addition of carbachol (Sigma-Aldrich) at 100 μM. The preloaded cell was individually selected from a field of view, and fluorescent intensity of Fluo-4 was measured and normalized to baseline over time (F/F0), in individual cells. The relative fluorescence unit of Fluo-4, AM intensity was analyzed by ZEN (blue edition) (Carl Zeiss AG).

TEM. Cells were washed with DPBS (Corning), detached with 0.25% Trypsin-EDTA (Gibco), and pre-fixed with Karnovsky's fixative containing 2% glutaraldehyde (Merck, Darmstadt, GE), 2% PFA (Merck), and 0.5% calcium chloride (Sigma-Aldrich) in 0.1 mole per liter of PBS (Sigma-Aldrich) overnight at 4° C. in the dark. The cells were washed with PBS for two hours and then fixed with 1% osmium tetroxide (Polysciences, Warrington, PA) in PBS for two hours. The post-fixed cells were washed with PBS for 10 minutes and gradually dehydrated through a series of ascending ethanol dilutions to absolute ethanol (Merck). The dehydrated cells were infiltrated with propylene oxide (Sigma-Aldrich) for 10 minutes and embedded with a Poly/Bed 812 (Luft formulations) Embedding Kit/DMP-30 (Polysciences). The embedded cells were then polymerized in a TD-700 electron microscope oven (DOSAKA, Kyoto, JP) at 65° C. for 12 hours. The blocks were cut into 200-nm semi-thin sections with a diamond knife in an Ultramicrotome Leica EM UC7 (Leica Microsystems, Wetzlar, GE), and the sections were stained with toluidine blue (Sigma-Aldrich). The region of interest was selected and cut into 80-nm thin sections using the ultramicrotome. The thin sections were then impregnated with 3% uranyl acetate (Polysciences) for 30 minutes and 3% lead citrate (Polysciences) for seven minutes, and then captured by a JEM-1011 transmission electron microscope (JEOL Ltd., Tokyo, JP) at the acceleration voltage of 80 kV equipped with a Megaview III CCD camera (Soft Imaging System GmbH, Münster, GE).

Scratch wound healing assay. Cells were seeded onto a 6-well plate at a density of 2.0×10⁵ cells per ml and incubated overnight at 37° C. with 5% CO₂. The confluent cell monolayer was scrapped in a straight line to create a “scratch” with a p200 pipette tip. The scratched fields were acquired before and after 24 hours under a phase-contrast microscope. The images were further analyzed by measuring the areas of the scratch closure using a Java-based image processing program, ImageJ (U.S. National Institutes of Health).

### Results

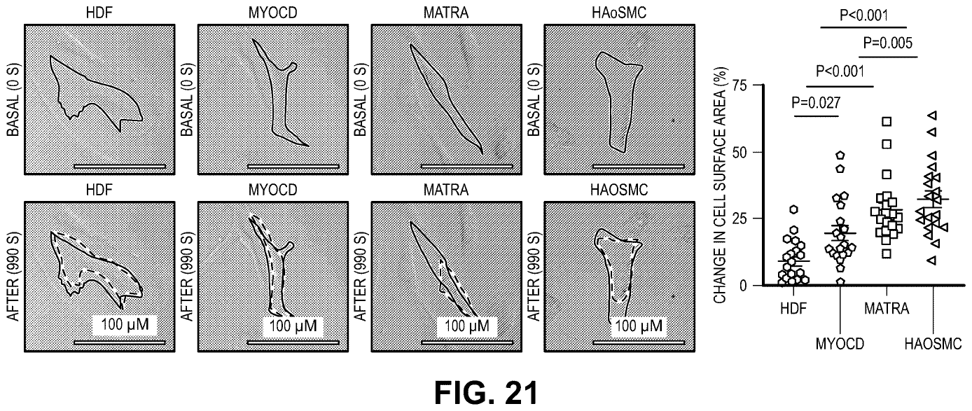

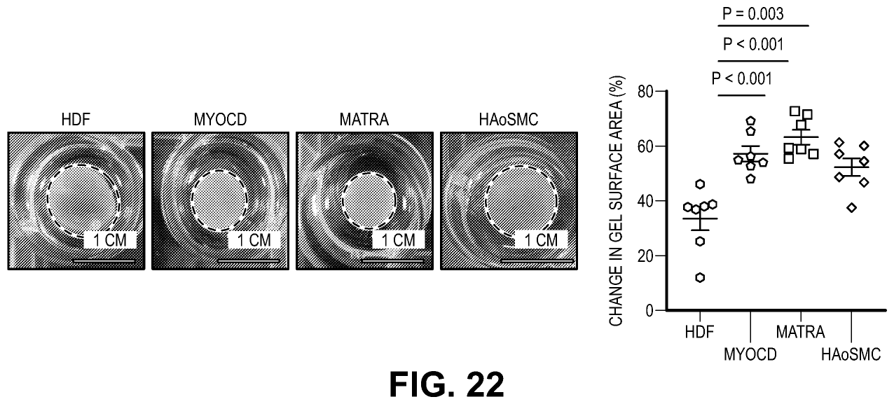

Contractility increased in MYOCD and MATRA-transduced HDFs. The most defining feature of contractile SMCs is the ability to contract, so the contractility of MATRA-treated HDFs was determined following stimulation with a vasoconstrictor, carbachol. Following carbachol treatment, the untransduced HDFs showed the lowest changes in cell surface area by contracting ~9%, whereas MYOCD-transduced HDFs contracted by ~20% (FIG. 21). However, contracted MATRA-treated HDFs exhibited a ~28% change of cell surface area, resembling the change seen in human aortic SMCs (HAoSMCs) (~32%) (FIG. 21). To further confirm their contractility, a carbachol-induced collagen gel contraction assay was performed (FIG. 22). Similar results were obtained in collagen gels embedded with these cells. MYOCD-transduced and MATRA-treated HDFs in gels displayed a much stronger contraction than did HAoSMCs or untransduced HDFs in gels (FIG. 22).

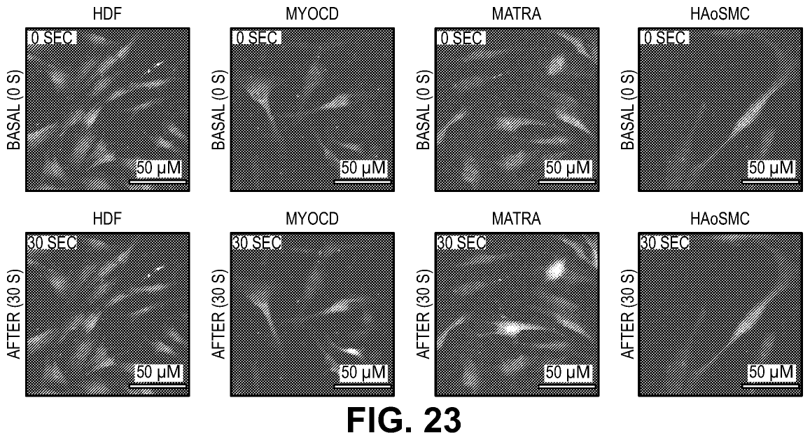

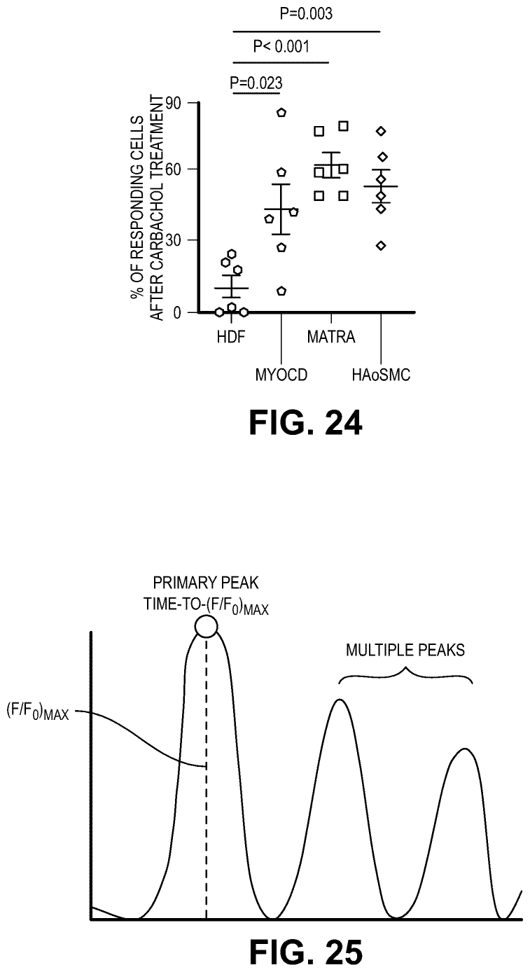

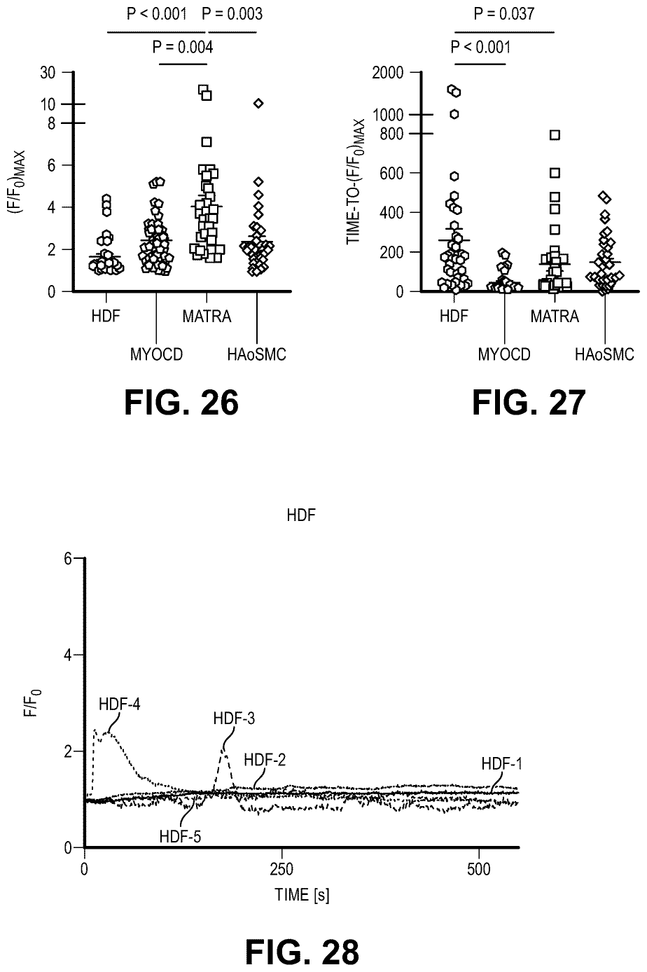

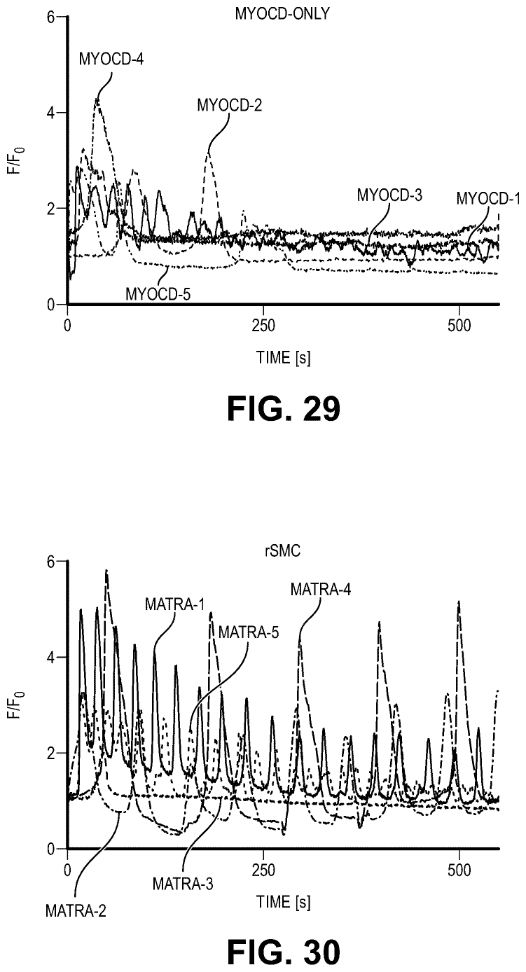

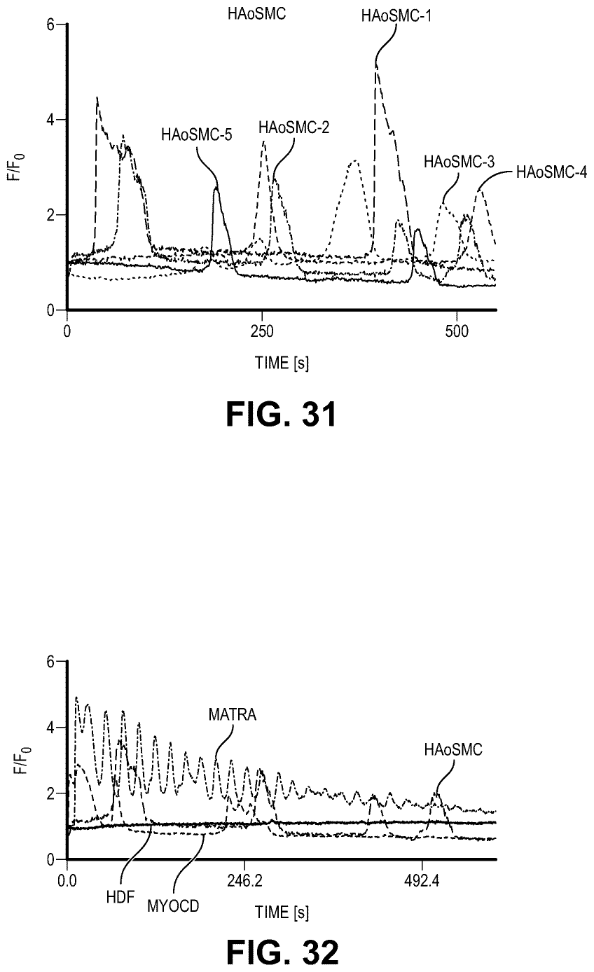

Intracellular calcium release increased in MATRA-transduced HDFs. Changes in the intracellular calcium (Ca²⁺) are central to the contractile function of SMCs (see, for example, Somlyo & Somylo, Nature, 460:705-710 (2009)). Upon carbachol treatment, the untransduced HDFs exhibited low intracellular Ca²⁺ release (FIG. 23). MYOCD-transduced and MATRA-treated HDFs showed higher intracellular Ca²⁺ release, which was similar to HAoSMCs (FIG. 23). Quantitative analysis of Fluo-4 intensity demonstrated a significantly higher response rate in MATRA-treated HDFs compared to other controls (MATRA, ~63%; HDF, ~11%; MYOCD-HDFs ~44%; HAoSMC, ~54%) (FIG. 24). As shown in FIG. 25, the maximum fluorescence intensity ((F/F0) max), and time to peak fluorescence (time-to-(F/F0)max) were determined. (F/F0) max in response to carbachol was significantly higher in MATRA-treated HDFs compared to all other control groups (FIG. 26). The time-to-(F/F0)max was significantly shorter for MYOCD-transduced HDFs and MATRA-treated HDFs compared to the untransduced HDFs (FIG. 27). The temporal characteristics of intracellular Ca²⁺ release in MATRA-treated HDFs were similar to HAoSMCs. Monitoring of F/F0 in a single cell over time further illustrated that individual cells from each group showed substantial variations in the number of Ca²⁺ events (FIGS. 28-31). The vast majority of untransduced HDFs produced no or one large primary peak upon carbachol treatment, whereas MYOCD-transduced HDFs and HAoSMCs often had a large primary peak, which was followed by a low number of recurrent peaks (FIG. 32). In contrast, MATRA-treated HDFs produced much more robust primary peaks and submaximal Ca²⁺ events in the form of propagated waves over an extended period (FIG. 32). Vasoconstrictor-induced propagated Ca²⁺ events have been demonstrated in in situ or freshly isolated SMCs (see, for example, Halaidych et al, Stem Cell Reports, 12:647-656 (2019); Blatter & Wier, Am. J. Physiol. 263 (576-586) (1992); Gordienko et al, J. Physiol., 507 (3) 707-720 (1998); Borysova et al., Cell Calcium, 54 (163-174) (2013)). Such events were reflected in MATRA-treated HDFs, suggesting stronger contractile properties than that of MYOCD-transduced HDFs.

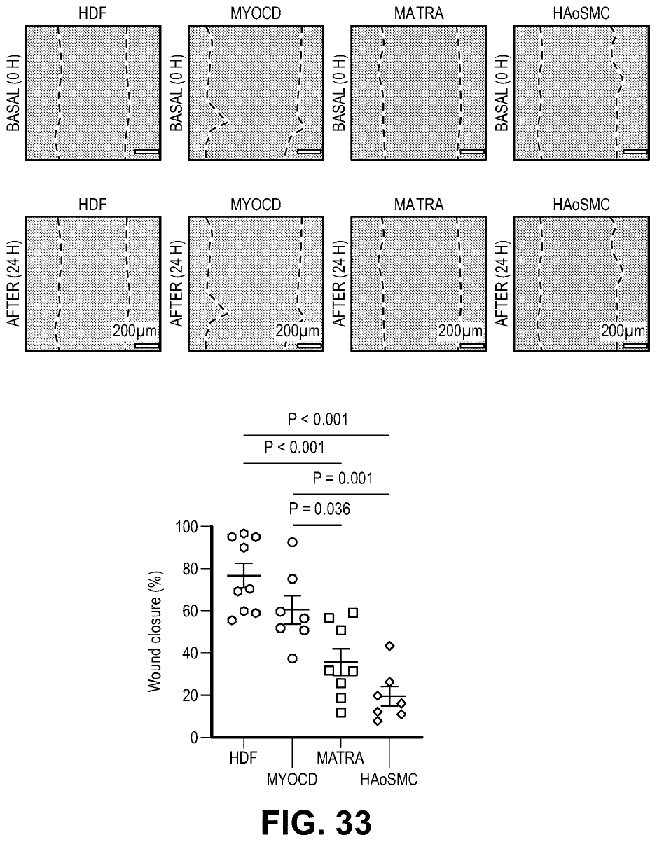

Cell migration decreased in MATRA-treated HDFs. Acquisition of the contractile SMC phenotype is often accompanied by a reduction in cell migration, assessed herein by a scratch wound healing assay. 24 hours post-scratch, the migratory distance measured by percent wound closure was significantly lower in MATRA-treated HDFs compared to the untransduced and MYOCD-transduced HDFs (FIG. 33).

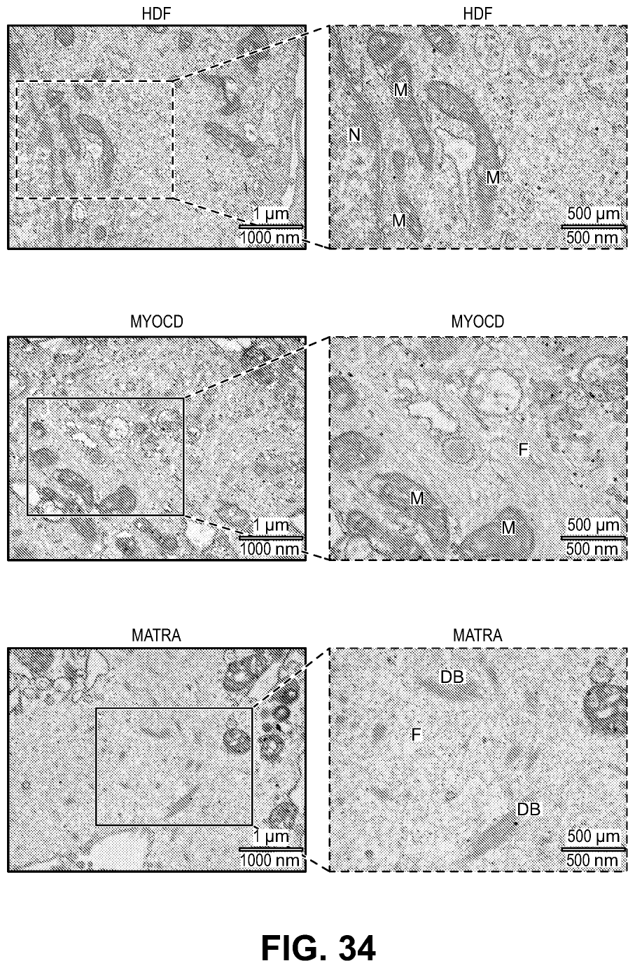

Cellular ultrastructure of MATRA-treated HDFs showed features of contractile SMCs. The cellular ultrastructure of the cytoskeletal and contractile apparatus of the cells was assessed by transmission electron microscopy (TEM) (FIG. 34). TEM showed that the untransduced HDFs contained abundant mitochondria and free ribosomes, important characteristics of fibroblasts (see, for example, Lucky et al., Exp. Cell Res. 92 (383-393) (1975)), but no discrete contractile filaments (FIG. 34). MYOCD-transduced HDFs, in contrast, exhibited reduced mitochondria and free ribosomes and increased contractile filaments in the absence of striations (FIG. 34). Notably, as shown in FIG. 34, MATRA-treated HDFs showed further increased contractile filaments, which are anchored by dense bodies, a key ultrastructural feature of contractile SMCs (see, for example, Ross, J. Cell Bio. 50:172-186 (1971)) in a typical non-striated SMC-like pattern. Moreover, there was substantial loss of mitochondria and ribosomes (FIG. 34).

### Example 3: Transcriptome Analysis Demonstrating Reprogramming of HDFs into Contractile SMCs

### Materials and Methods

Bulk RNA Sequencing (RNA-seq) Analysis. Total RNA was obtained from two biological replicates of each group (HAoSMC, HDF, MYOCD-only cells, and MATRA-treated cells) using the miRNeasy Mini Kit (QIAGEN), according to the manufacturer's instructions. The sample integrity and concentration were assessed by Agilent 2100 BioAnalyzer (Agilent Technologies, Inc., Santa Clara, CA), according to the manufacturer's instructions, and only samples with an RNA Integrity Number value of higher than 8 were used. Polyadenylation (poly(A)) of mRNA was enriched by magnetic beads with oligo (deoxythymine) (oligo (dT)) and then cut into short fragments. The cDNA was subject to end-repair and poly(A) tailing and connected with sequencing adapters using TruSeq Stranded mRNA Sample Prep Kit (Illumina, Inc., San Diego, CA), according to the manufacturer's instructions. The libraries whose sizes ranged between 120-200 base pairs (bps) were then subject to paired-end sequencing with a 150-bp read length using an Illumina NovaSeq 6000 (Illumina, Inc.) platform, yielding an average of 27 million reads per library, as shown in Table 3. The raw reads were processed for quality assessment, and only clean reads for each sample were further analyzed.

TABLE 3
Sequencing data used in bulk RNA-seq analysis

| | | | | | Uniquely |
|---|---|---|---|---|---|
| | Sample | Raw | Clean | Mapped | mapped |
| Sample | ID | reads | reads (%) | reads (%) | reads (%) |
| HDF | HDF-1 | 25,214,804 | 25,046,142 | 23,999,908 | 22,883,520 |
| | | | (99.33%) | (95.18%) | (90.75%) |
| | HDF-2 | 26,347,038 | 26,160,866 | 25,977,556 | 23,819,216 |
| | | | (99.29%) | (94.8%) | (90.41%) |
| MYOCD- | MYOCD- | 28,780,464 | 28,468,716 | 26,761,688 | 25,494,258 |
| only | ONLY-1 | | (98.92%) | (92.99%) | (88.58%) |
| | MYOCD- | 25,399,052 | 25,151,258 | 23,646.328 | 22,486,404 |
| | ONLY-2 | | (99.02%) | (93.1%) | (88.53%) |
| rSMC | rSMC-1 | 32,118,116 | 31,886,276 | 30,469,642 | 29,114,344 |
| | | | (99.28%) | (94.87%) | (90.65%) |
| | rSMC-2 | 29,978,450 | 29,745,490 | 28,112,690 | 26,830,286 |
| | | | (99.22%) | (93.78%) | (89.5%) |
| HAoSMC | HAoSMC-1 | 26,815,774 | 26,600,466 | 25,224,316 | 23,992,262 |
| | | | (99.20%) | (94.07%) | (89.47%) |
| | HAoSMC-2 | 28,780,464 | 28,468,716 | 26,746,818 | 25,562,310 |
| | | | (99.42%) | (95.03%) | (90.83%) |

Bioinformatics Analysis. Reads were pre-processed and filtered by eliminating low-quality reads and adapter sequences using Cutadapt (open-source software developed by Marcel Martin, available under the MIT license). The filtered reads were aligned to the reference genome of Genome Reference Consortium Human Build 38 (GRCh38; hg38) by STAR (open-source software developed by Alexander Dobin et al., under GPLv3 license). The gene expression levels were then estimated using featureCounts (open-source software developed by Yang Liao et al., available under the GNU General Public License) with a set of default parameters. Differentially expressed gene (DEG) analysis between the groups were analyzed using DESeq2 (open-source software developed by Michael Love et al., available on the Bioconductor platform), with the gene information cutoff set at p-value of <0.05 and absolute log fold change value of >2.0, as shown in Table 4. Bioinformatics analysis was additionally conducted with iDEP.91 (software developed by Steven Ge et al., available on the Bioconductor platform). Defined sets of genes of the HDFs and rSMCs used for gene set enrichment analysis (GSEA) were defined by more than two-fold gene expression change. Enrichment of these gene sets was evaluated by GSEA software (v4.1.0; The Broad Institute, Cambridge, MA) with 1,000 permutations to gene set, no dataset collapse, and use of weighted enrichment statistics.

TABLE 4
Differentially expressed genes in rSMCs

| | Official | rSMC | rSMC | rSMC |
|---|---|---|---|---|
| | gene | v HDF | v HDF | v HDF |
| Rank | symbol | logFC | P-val | FDR |
| 1 | MYOCD | 11.03606575 | 2.10293E−13 | 6.86612E−10 |
| 2 | ACTA1 | 11.02419982 | 2.17128E−13 | 6.86612E−10 |
| 3 | MYH11 | 10.99436658 | 1.13033E−10 | 3.66602E−08 |
| 4 | ACTG2 | 8.991302245 | 1.24375E−3 | 6.86612E−10 |
| 5 | NPPB | 8.946331503 | 4.94374E−11 | 2.48665E−08 |
| 6 | MGP | 8.751735553 | 1.18705E−11 | 1.155E−08 |
| 7 | IRS4 | 8.008083736 | 8.8164E−08 | 5.06903E−06 |
| 8 | DHRS3 | 7.564377085 | 2.37749E−12 | 3.7591E−09 |
| 9 | ACTA2 | 7.381706647 | 5.13664E−14 | 6.49733E−10 |
| 10 | HOXB9 | 7.325216078 | 3.29237E−07 | 1.39749E−05 |
| 11 | CA2 | 7.247259969 | 6.73878E−08 | 4.19896E−06 |
| 12 | SORBS1 | 6.931560968 | 2.02784E−09 | 3.95637E−07 |
| 13 | MYL7 | 6.678073638 | 5.07765E−11 | 2.48665E−08 |
| 14 | HOXB6 | 6.651720051 | 4.24151E−07 | 1.66102E−05 |
| 15 | CBSL | 6.612686791 | 2.53787E−06 | 6.88874E−05 |
| 16 | CITED1 | 6.559067956 | 4.16281E−07 | 1.64035E−05 |
| 17 | SLAIN1 | 6.343228112 | 2.7794E−07 | 1.2601E−05 |
| 18 | TINAGL1 | 6.33011891 | 1.00638E−12 | 1.81853E−09 |
| 19 | TACSTD2 | 6.262370618 | 5.62291E−09 | 7.99148E−07 |
| 20 | CKMT1B | 6.250991859 | 3.82167E−06 | 9.55792E−05 |
| 21 | PLN | 6.170759268 | 3.69527E−07 | 1.50667E−05 |
| 22 | CDH8 | 6.126183561 | 3.77237E−08 | 3.10379E−06 |
| 23 | GPC3 | 6.102859053 | 1.00471E−06 | 3.30797E−05 |
| 24 | KCNQ2 | 6.087039522 | 5.58491E−06 | 0.000131798 |
| 25 | CYP26A1 | 6.06233012 | 2.63541E−06 | 7.09261E−05 |
| 26 | BEX2 | 6.053534085 | 9.32532E−08 | 5.31333E−06 |
| 27 | DIPK1B | 6.00851342 | 1.4505E−07 | 7.45825E−06 |
| 28 | CLUL1 | 5.988539571 | 2.01636E−07 | 9.62452E−06 |
| 29 | NMU | 5.937248358 | 5.63785E−06 | 0.000132307 |
| 30 | ZIC2 | 5.905976923 | 5.91392E−07 | 2.162E−05 |

### Results

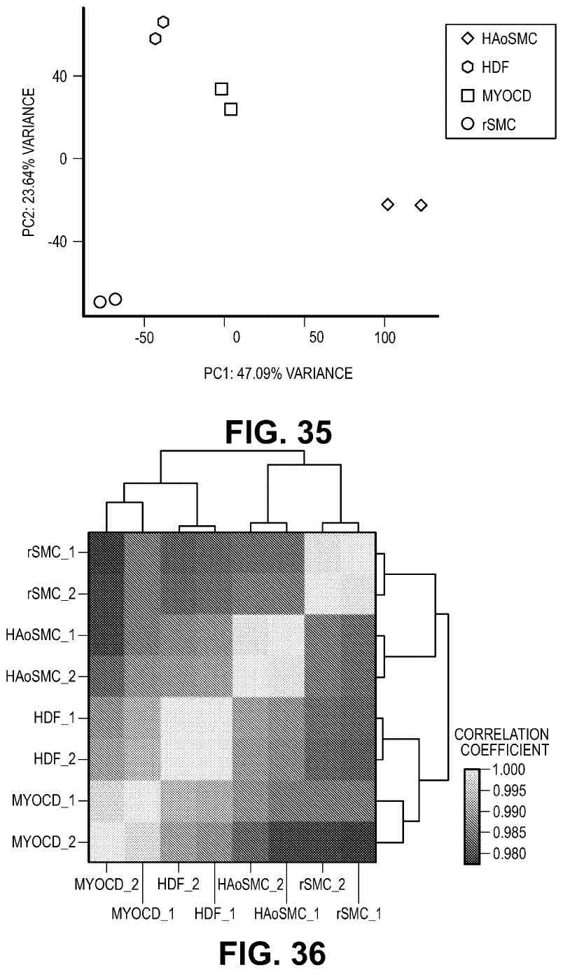

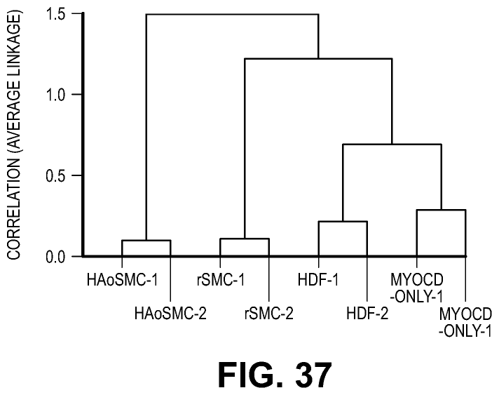

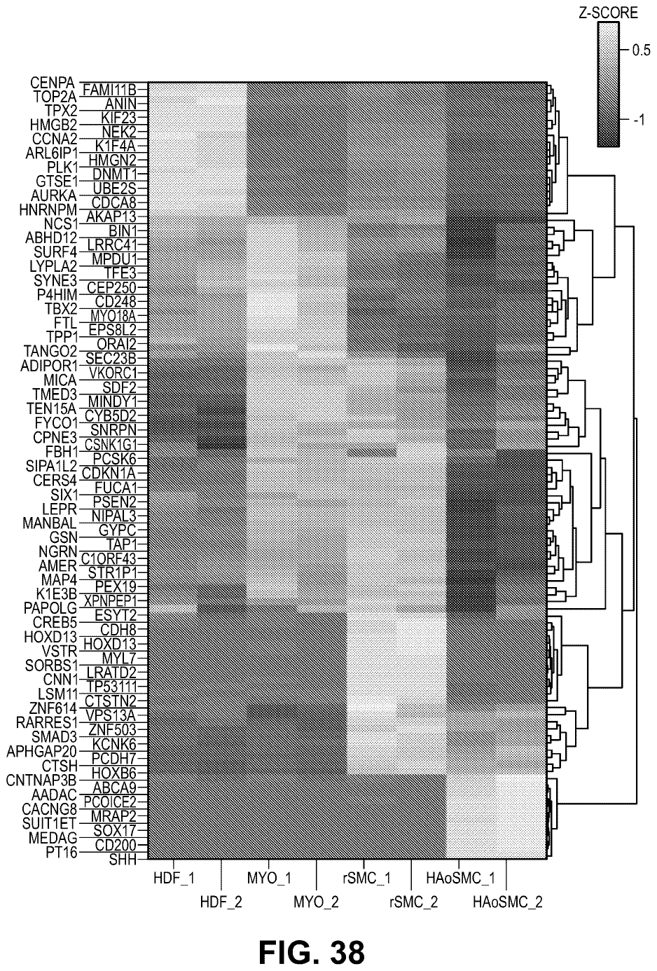

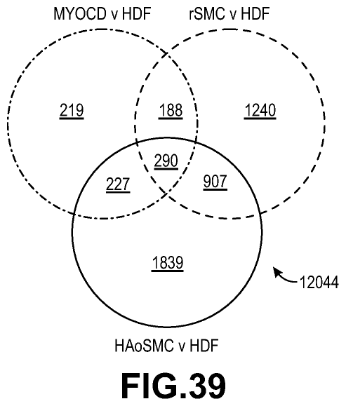

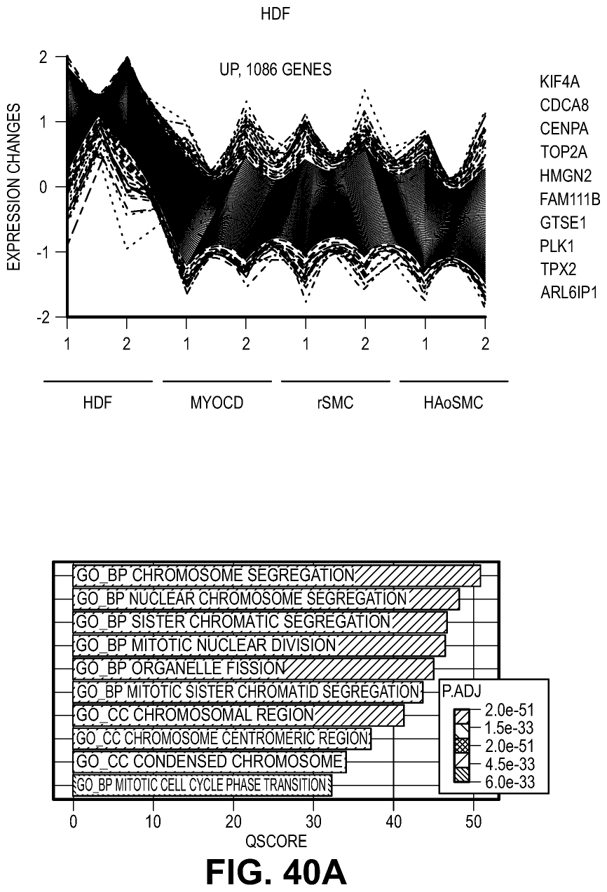

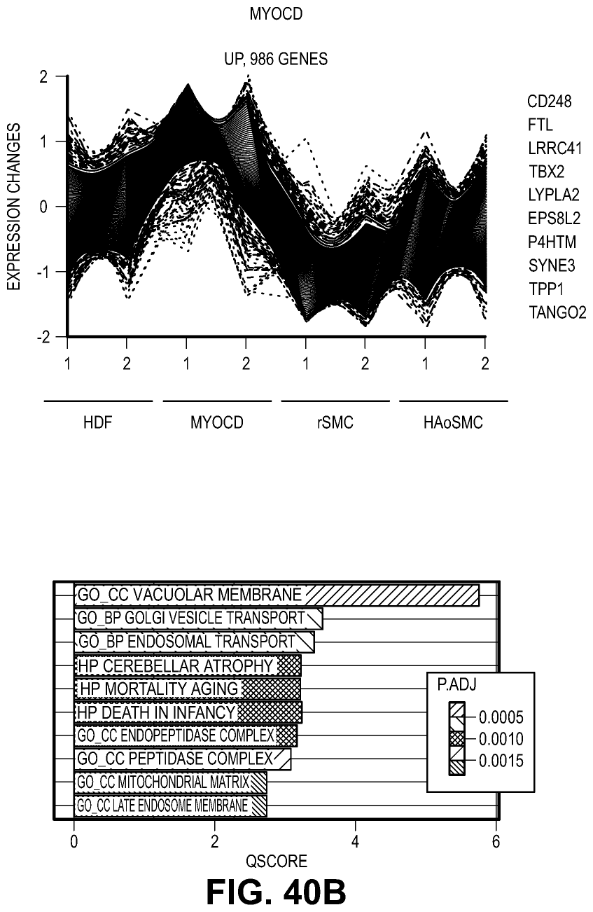

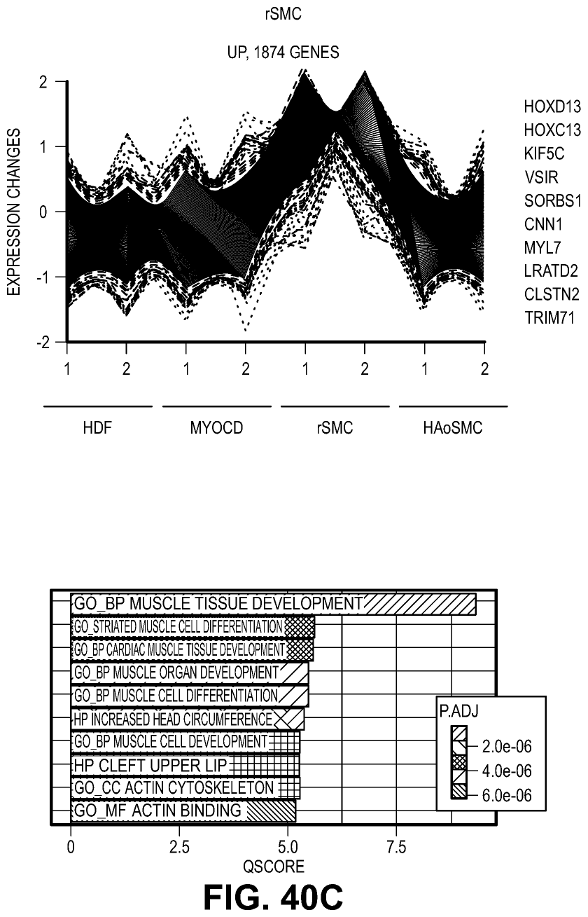

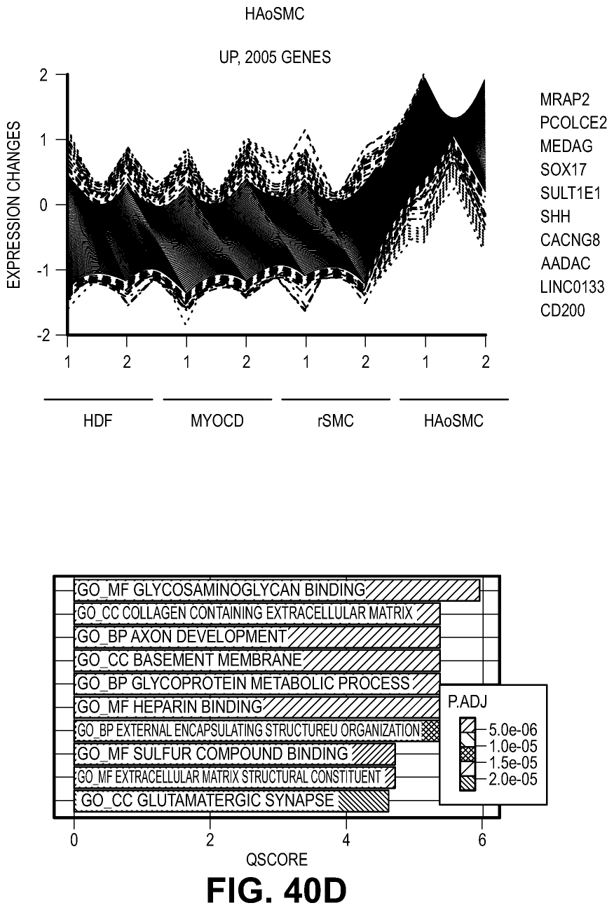

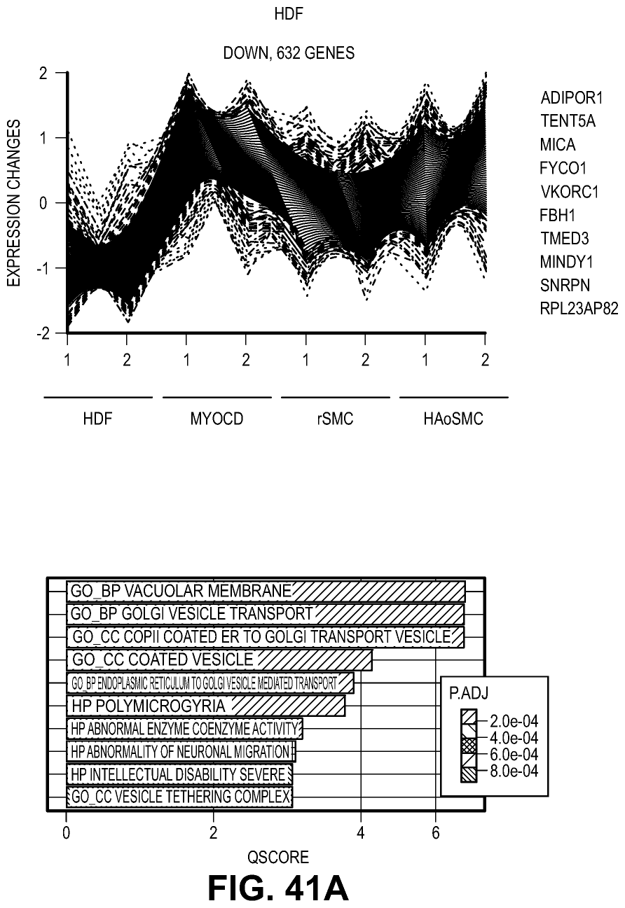

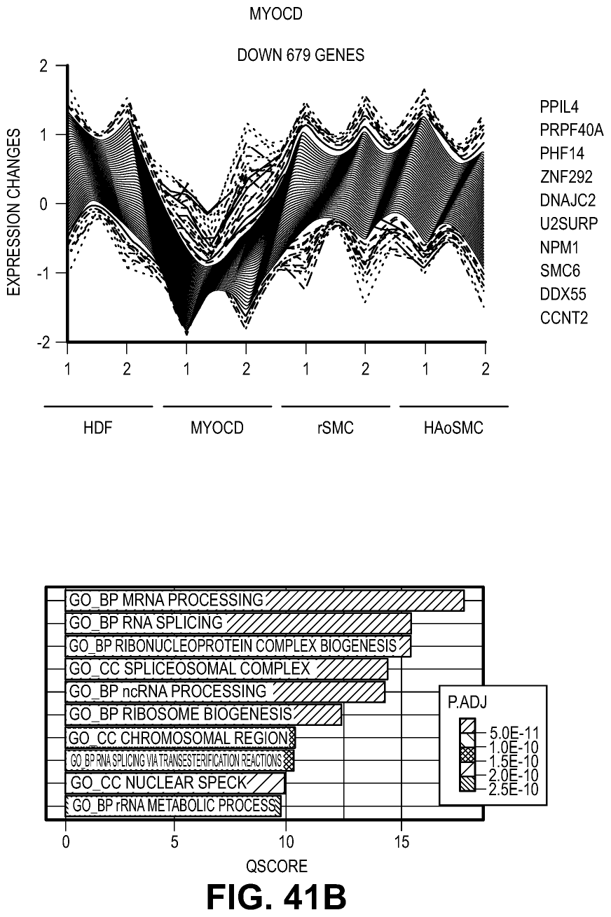

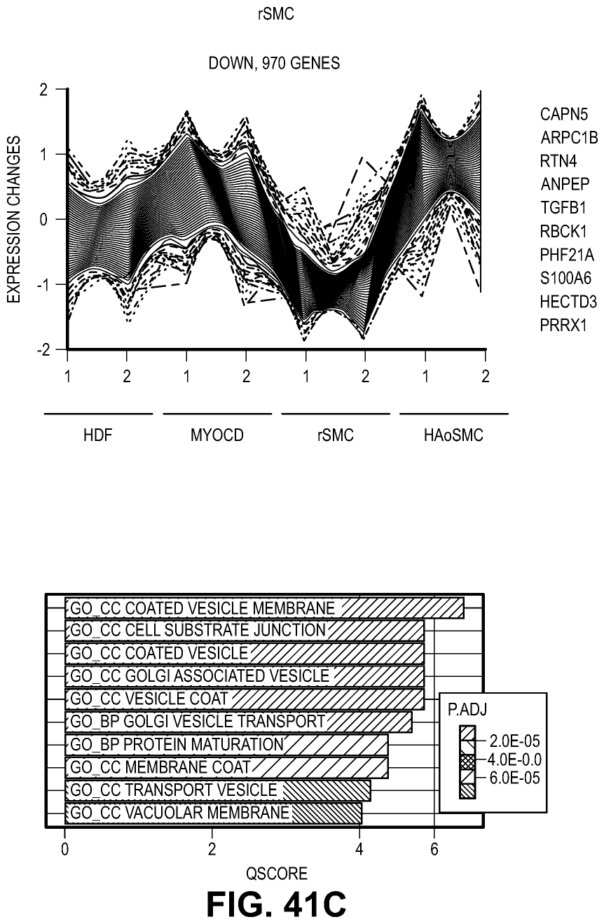

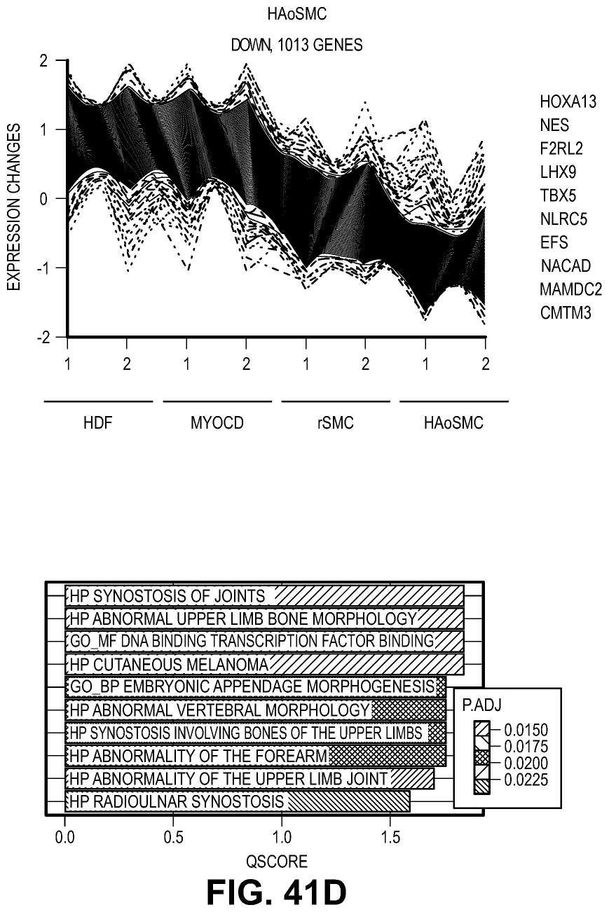

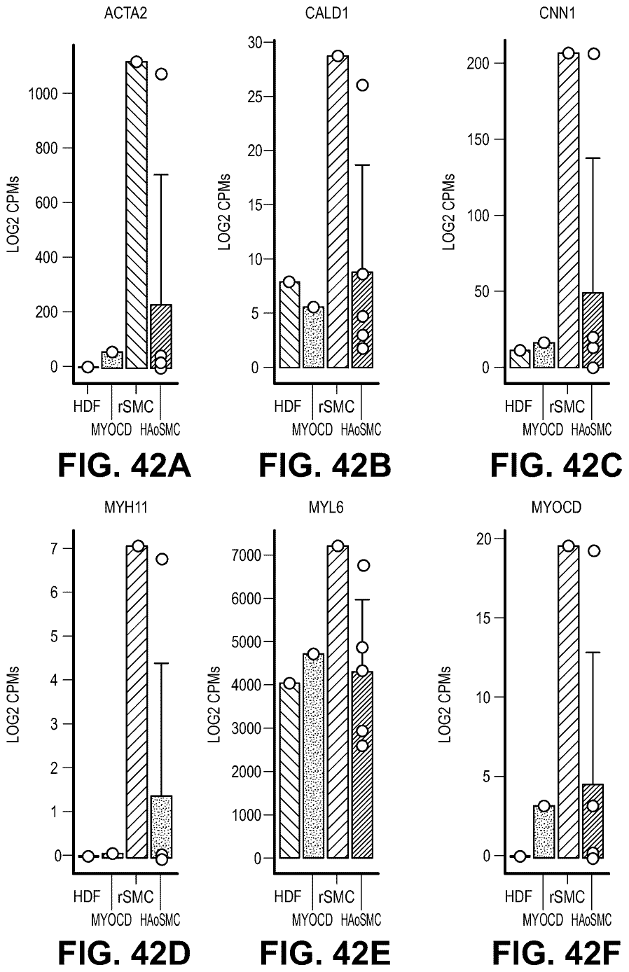

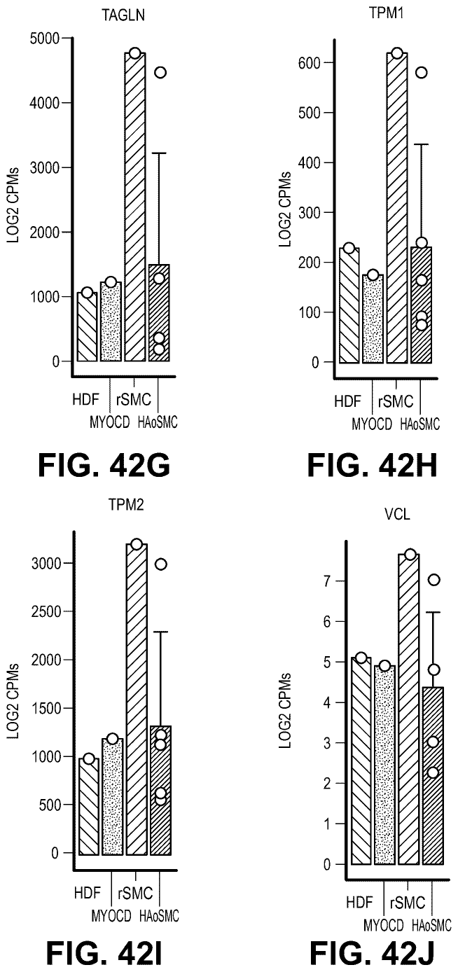

SMC gene expression in MATRA-treated HDFs. To decipher the cell fate transition during reprogramming, RNA-seq was conducted using total RNAs from HDFs, MYOCD-only cells, rSMCs, and HAoSMCs. Principal component analysis (PCA) showed four distinct groups (FIGS. 35-36). Although MYOCD-only cells showed the gene expression patterns similar to those of HDFs, rSMCs showed extensively different transcriptomic patterns (FIGS. 37-39). Hierarchical clustering analysis revealed that rSMCs and HAoSMCs were closely positioned and distinguished from HDFs and MYOCD-only cells (FIG. 38). To further investigate distinguishable gene expression patterns for these cell populations, soft clustering was applied to RNA-seq data analysis. The results showed that more than 1000 genes were enriched and ~600 to 2000 genes were downregulated in each population (FIG. 40-41). As shown in FIG. 40, HDFs highly expressed KIF4A, CDCA8, CENPA, and GTSE1, which are associated with mitosis and cell proliferation (see, for example, Ultera et al., EMBO J., 17:5015-5025 (1998); Howman et al., Proc. Natl. Acad. Sci., 97:1148-1153 (2000); Mazumdar & Misteli, J. Cell Bio., 166 (613-620) (2004); Sampath et al., Cell, 118:187-202 (2004)). MYOCD-only cells expressed high levels of the endolysosomal genes (LYPLA2, TPP1, and TANGO2) and cytoskeletal genes (EPS8L2 and SYNE3) (FIG. 40). rSMCs were enriched with the genes involved in muscle development and function (SORBS1, CNN1, and MYL7) and embryonic skeletal system morphogenesis (HOXD13 and HOXC13) (FIG. 40). Cultured HAoSMCs highly expressed genes related to signal transduction systems (MRAP2, SULTIE1, CACNG8, and CD200) (FIG. 40). Gene ontology (GO) analyses were in line with these results (FIG. 40). The expression of contractile SMC marker genes (ACTA2, CALD1, CNN1, MYH11, MYL6, MYOCD, TAGLN, TPM1, TPM2, and VCL) were further compared between the four groups and rSMCs showed substantially higher levels of these genes (FIG. 42). Together, these RNA-seq analyses demonstrate that rSMCs are enriched with SMC genes, particularly contractile ones.

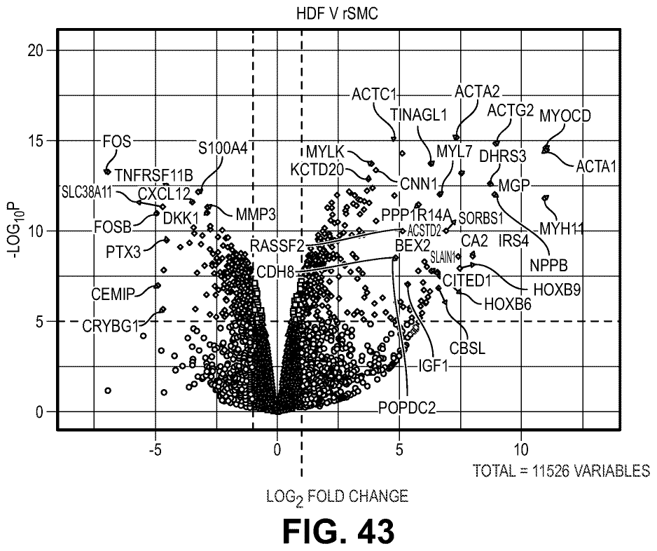

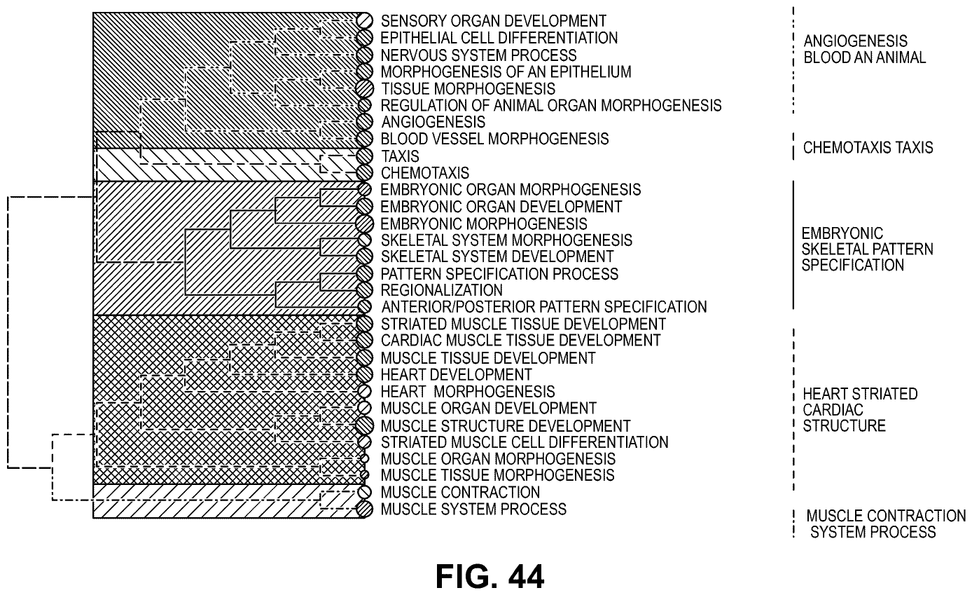

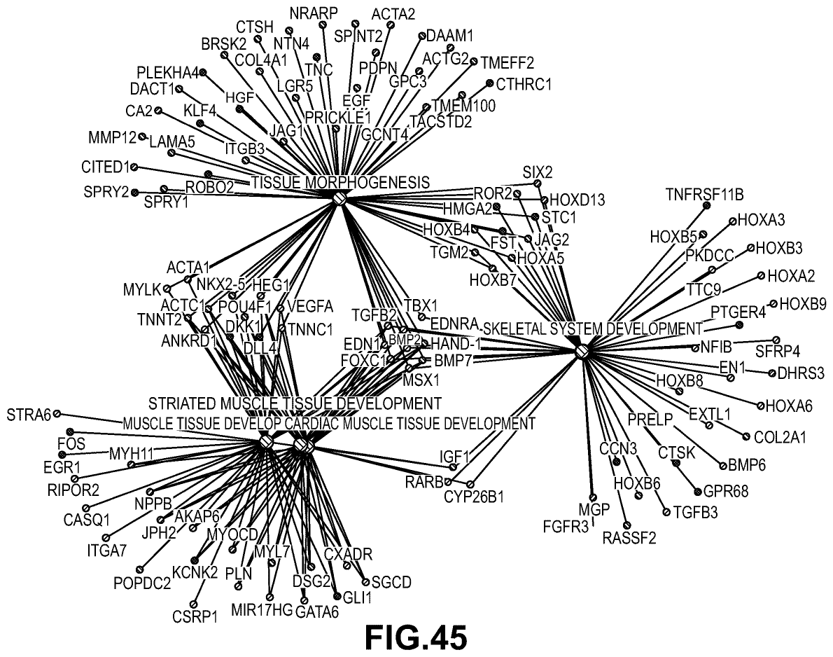

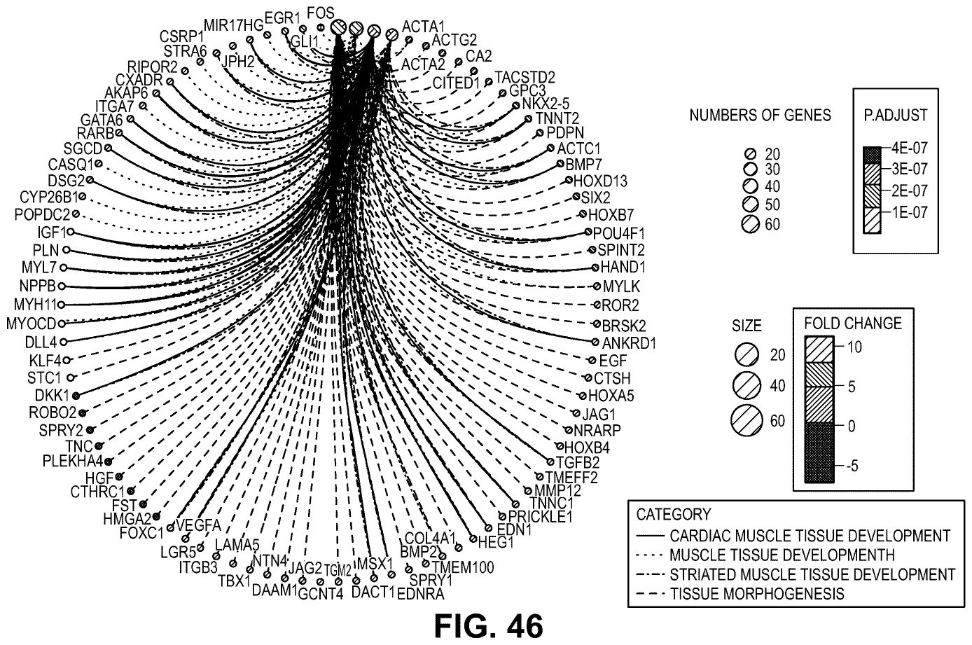

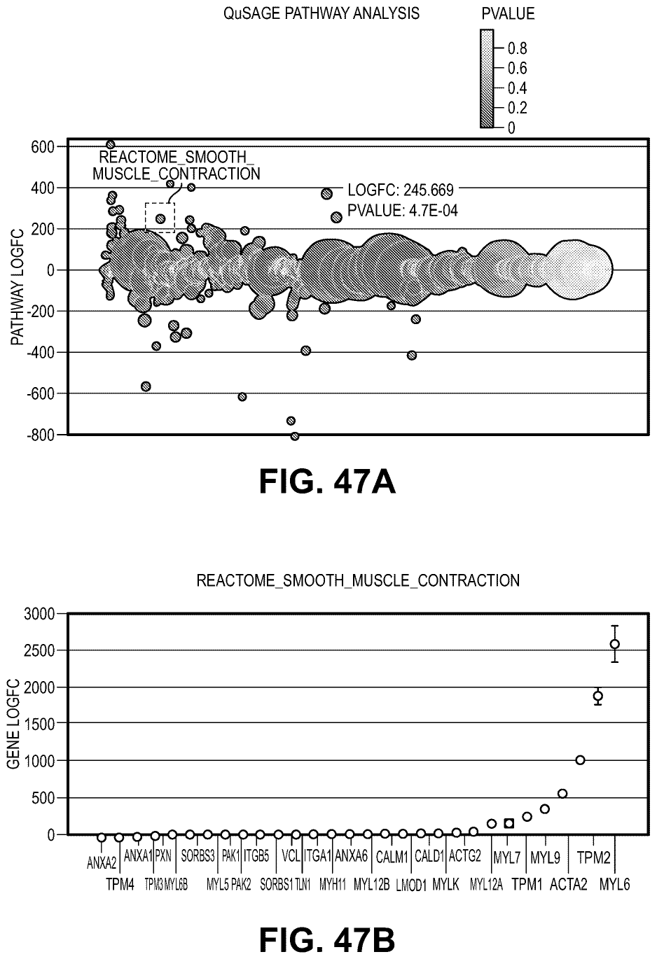

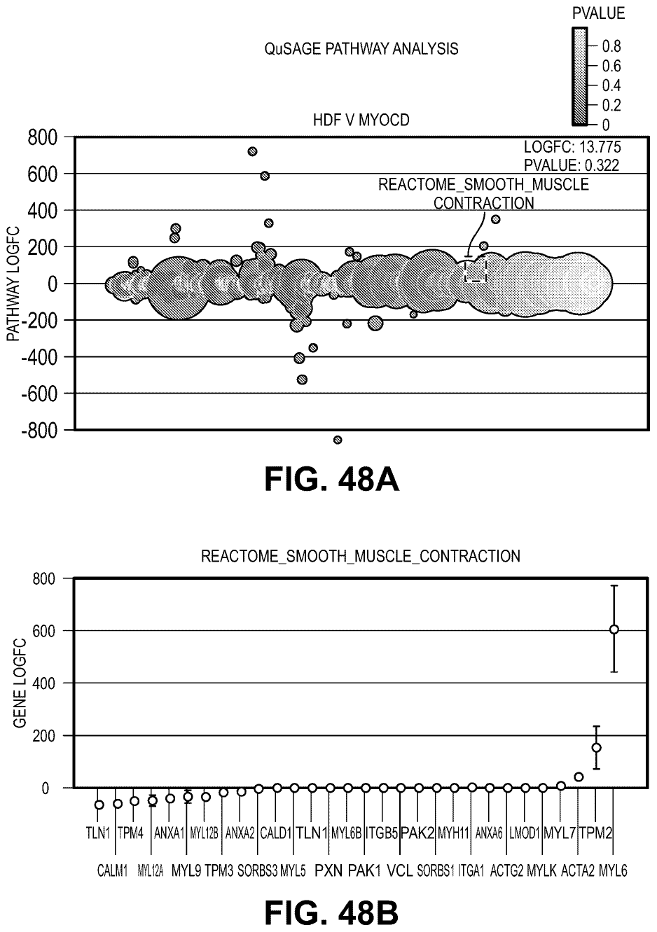

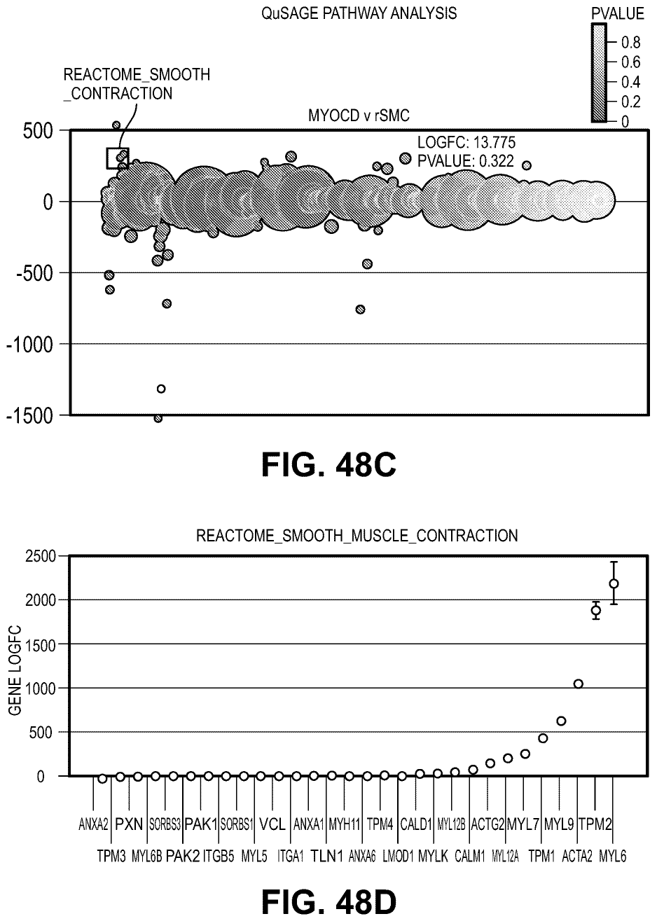

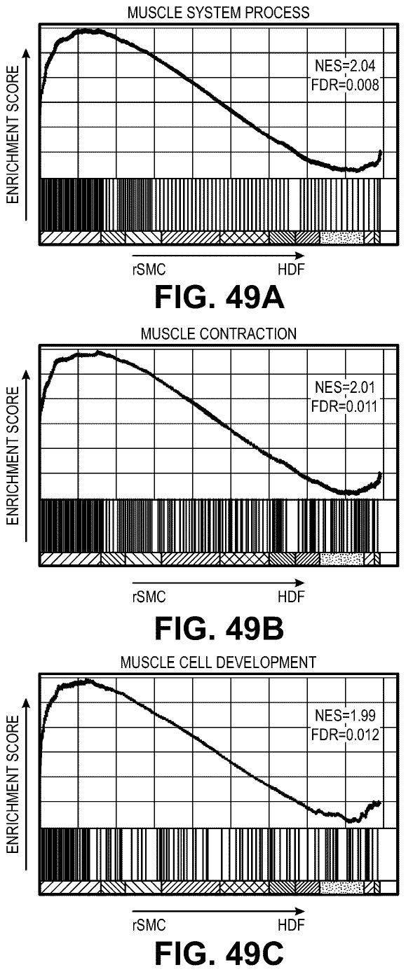

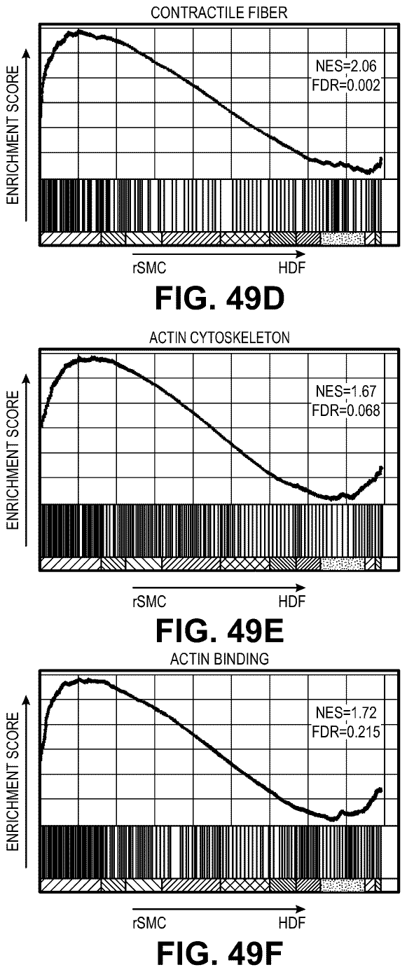

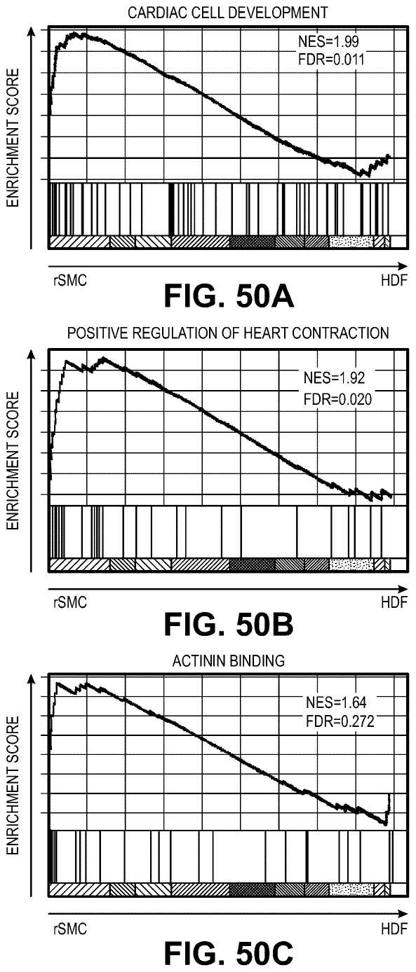

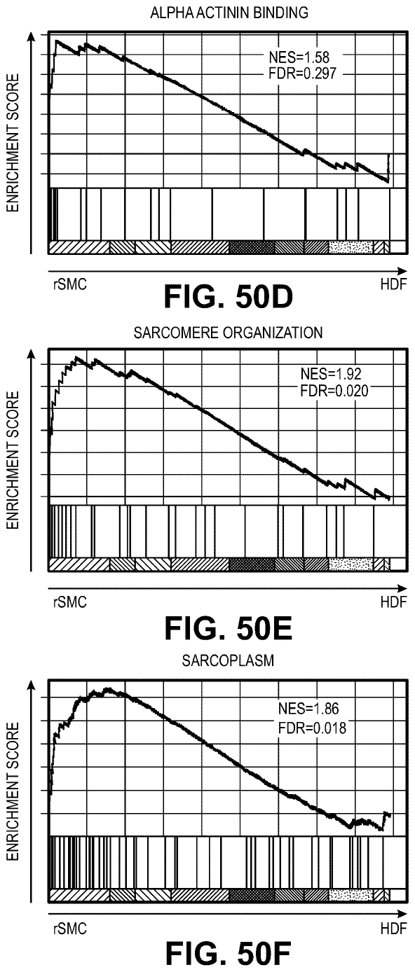

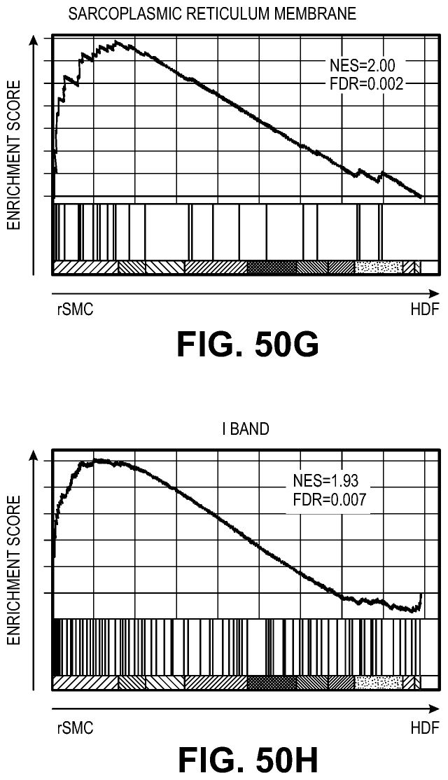

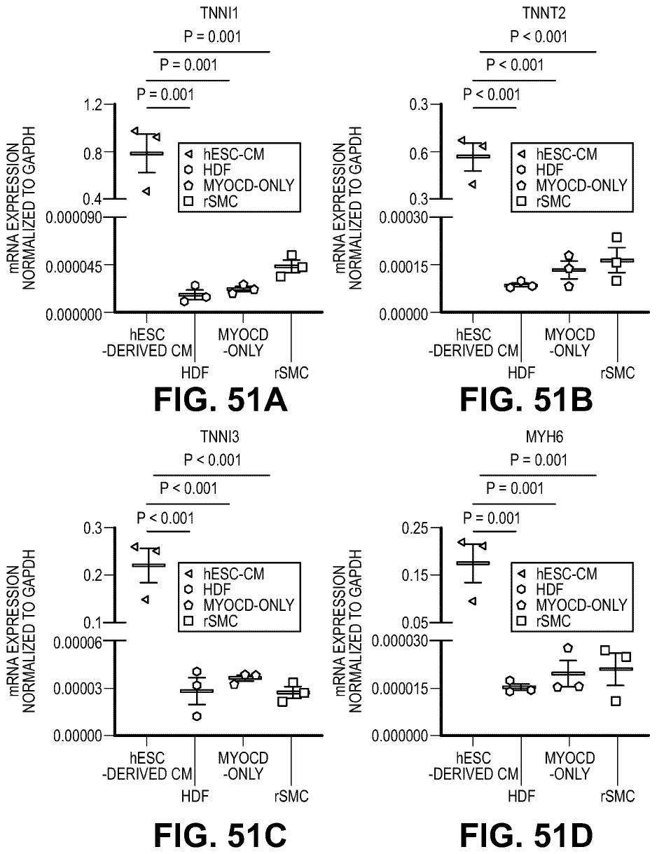

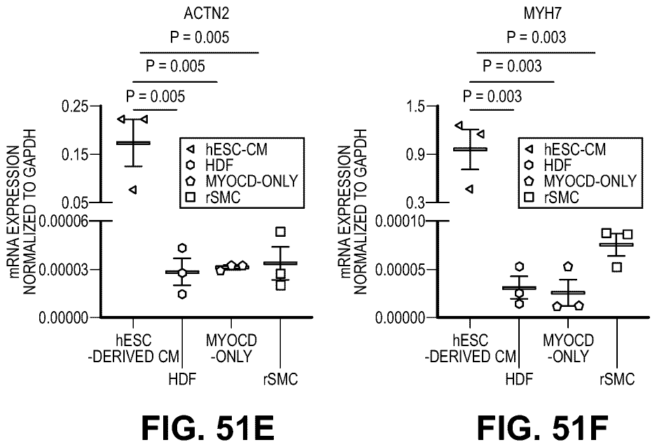

rSMCs display molecular characteristics of a contractile SMC phenotype. To reconstruct the reprogramming from HDFs to rSMCs, differentially expressed genes (DEGs) for these two groups were identified by using a threshold of false discovery rate of less than 0.1 and a fold-change of more than 2.0, as shown in Table 4. Among the genes most upregulated in rSMCs were encoding contractile and structural proteins of SMCs (ACTA1 and 2, ACTG2, CNN1, MYH11, MYLK, and MYL7), as well as their SMC-fate determining transcription factor, MYOCD (FIG. 43). The GO terms enriched in rSMCs were categorized into angiogenesis, chemotaxis, embryonic skeletal system morphogenesis and development, striated and cardiac muscle tissue development, and muscle tissue development and contraction (FIG. 44). The genes involved in these terms were depicted in FIGS. 45-46. Some of the enriched genes in rSMCs were ACTA1 and 2, ACTG2, MYH11, TPM1, MYLK, and MYL7 and 9, representing contractile SMCs (FIG. 43). Furthermore, Quantitative Set Analysis of Gene Expression (QuSAGE) was performed to unravel differentially expressed signaling pathways. Among these signaling pathways, smooth muscle contraction was specifically selected to measure a log₂-fold change (log FC) between HDFs and rSMCs (FIG. 47). The log FC was 245.7, which was significantly higher than were the log FCs between HDFs and MYOCD-only cells (13.8) and between MYOCD-only cells and rSMCs (231.9) (FIG. 48). GSEA demonstrated an upregulation of genes for muscle system process, muscle contraction, muscle cell development, contractile fiber, actin cytoskeleton, and actin binding, which are all associated with the function of contractile SMCs (FIG. 49). GSEA further showed enrichment of cardiac genes (NKX2.5, TNNT1 and 2, GATA6, HAND1, and MYL7) and biological processes associated with heart (cardiac cell development, positive regulation of heart contraction, actinin binding, alpha actinin binding, sarcomere organization, sarcoplasm, sarcoplasmic reticulum membrane, and I band) since MYOCD is a well-known cardiac transcription factor (see, for example, Wang et al., Cell, 105:851-862 (2001)) (FIG. 50). As shown in FIG. 51, gene expression of cardiomyocyte (CM) marker genes (TNNI1 and 3, ACTN2, TNNT2, and MYH6 and 7) were further examined in comparison with human embryonic stem cell-derived CMs by qRT-PCR. While MYOCD alone or in combination with ATRA marginally induced CM genes, the levels were quite low compared to hESC-CMs (FIG. 51) and did not show other CM characteristics. Collectively, these findings further support that rSMCs display molecular characteristics of a contractile SMC phenotype.

### Example 4: Effect of rSMC on Recovery from Tissue Ischemia

### Materials and Methods

Induction of hindlimb ischemia and cell transplantation. Hindlimb ischemia was performed on 8- to 10-week-old athymic male nude mice (Japan Shizouka Laboratory Center (SLC), Inc., Shizuoka, JP). The femoral artery was ligated and large branches were cauterized. Mice were then randomly assigned to four groups: surgery only (HLI), HDF−, MYOCD-only-cell-, or rSMC-injected groups (HLI+HDF, HLI+MYOCD, and HLI+rSMC). To determine the therapeutic effects, 2×10⁵ cells in 100 μl of DPBS (Corning) were intramuscularly injected into three sites of ischemic hindlimbs. The cells were pre-labeled with chloromethylbezamido (CellTracker™ CM-DiI; DiI; Invitrogen, Carlsbad, CA) before injection to monitor cellular behavior in tissues.

Blood flow measurement in hindlimbs. Blood flow of the hindlimbs was measured with a laser Doppler perfusion imager (Moor Instruments Ltd., Axminister, UK) after surgery and every week for four weeks. Mean values of perfusion were calculated from the stored digital color-coded images. The blood flow level of the ischemic limb was normalized to the non-ischemic limb to assess tissue function and to avoid data variations caused by ambient light and temperature, as shown in Table 5.

TABLE 5
Measurement of blood perfusion by
laser Doppler perfusion imaging

| | | Post- | | | | | |
|---|---|---|---|---|---|---|---|
| Group | n | surgery | Day 3 | Day 7 | Day 14 | Day 21 | Day 28 |
| Surgery | 9 | 0.037 ± | 0.073 ± | 0.151 ± | 0.220 ± | 0.253 ± | 0.331 ± |
| only | | 0.002 | 0.027 | 0.033 | 0.041 | 0.038 | 0.035 |
| HDF | 10 | 0.038 ± | 0.083 ± | 0.197 ± | 0.284 ± | 0.335 ± | 0.432 ± |
| | | 0.002 | 0.025 | 0.048 | 0.030 | 0.031 | 0.051 |
| MYOCD- | 9 | 0.046 ± | 0.100 ± | 0.279 ± | 0.354 ± | 0.383 ± | 0.514 ± |
| only | | 0.004 | 0.021 | 0.051 | 0.060 | 0.066 | 0.056 |
| rSMC | 10 | 0.048 ± | 0.085 ± | 0.253 ± | 0.400 ± | 0.431 ± | 0.674 ± |
| | | 0.004 | 0.016 | 0.021 | 0.037 | 0.038 | 0.040 |

Quantitative analysis of vascular functionality in ischemic tissues. Quantitative analysis of vascular functionality in ischemic tissues was performed using AngioTool (open-source software developed by Zudaire et al., available under the GNU General Public License). Four weeks post-transplantation, mice (Japan SLC, Inc.) were first anesthetized and intravenously injected with Fluorescein Griffonia simplicifolia lectin, isolectin B4 (ILB4; Vector Laboratories Inc., Burlingame, CA). The hindlimb muscles were removed, fixed in 4% PFA overnight at 4° C. in the dark, and incubated in 30% sucrose (Sigma-Aldrich) solution overnight at 4° C. in the dark. The tissues were embedded in Optimal Cutting Temperature (OCT) compound (Sakura Finetek USA, Torrance, CA) and sectioned in thickness ranging from 8 to 50 μm using a Leica CM1860 cryomicrotome (Leica Biosystems Nussloch GmbH, Nussloch, GE). Five to eight tissue sections with a range of thickness between 25 and 30 μm for each animal were randomly selected, counter-stained with DAPI (Invitrogen), and processed for analysis with a Zeiss LSM 700 or 780 confocal microscope (Carl Zeiss AG). Vascular functionality was then calculated using AngioTool software from at least twenty randomly selected fields.

Limb loss score index. Four weeks after induction of hindlimb ischemia, mice (Japan Shizuoka Laboratory Center (SLC), Inc., Shizuoka, Japan) were subject to euthanasia, and the ischemic limbs were captured by a digital camera and evaluated by the following scoring for assessment of ischemic hindlimb damage: 0=no necrosis; 1=tip necrosis; 2=toe necrosis; 3=foot necrosis; 4=leg necrosis; and 5=whole limb loss.

### Results

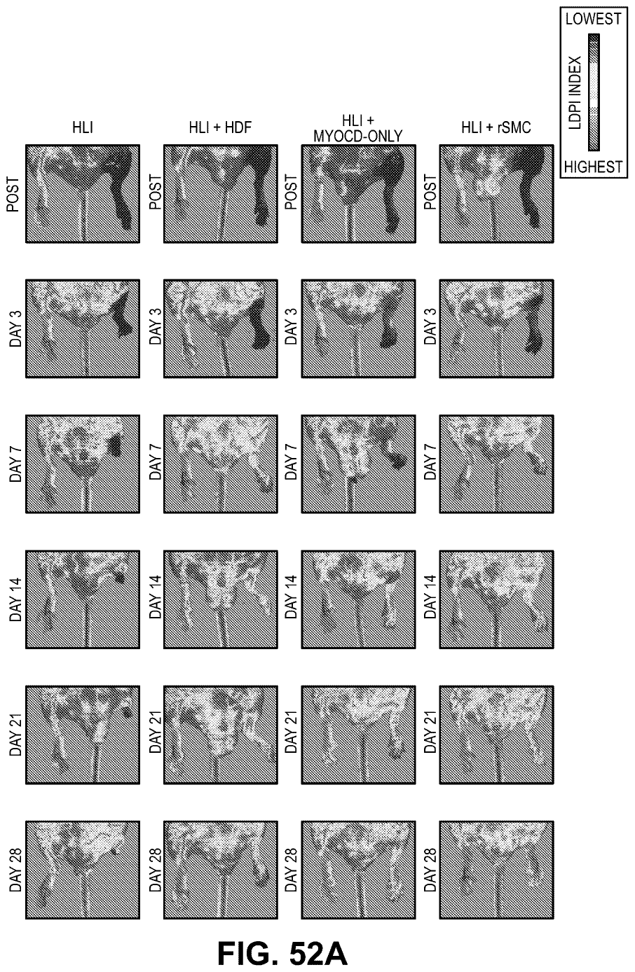

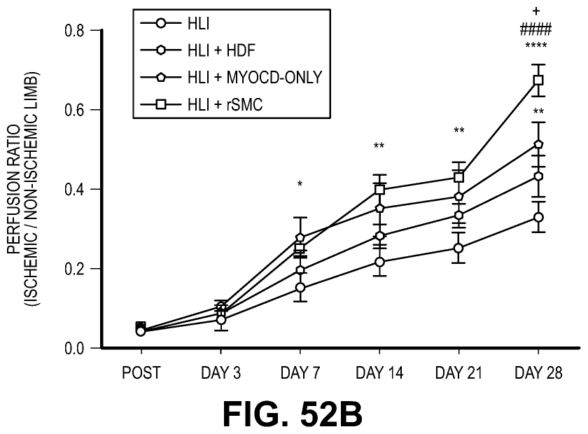

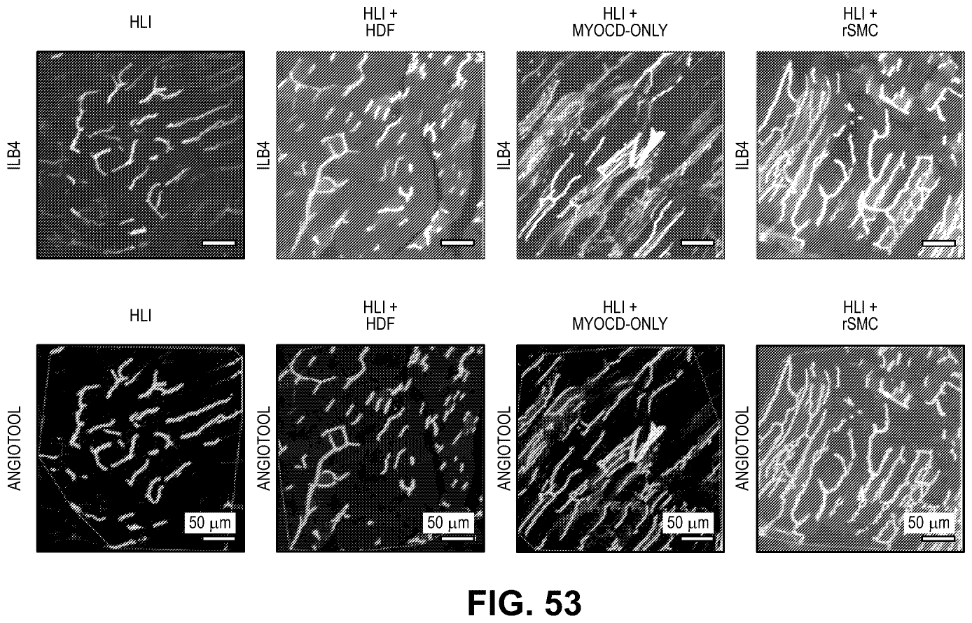

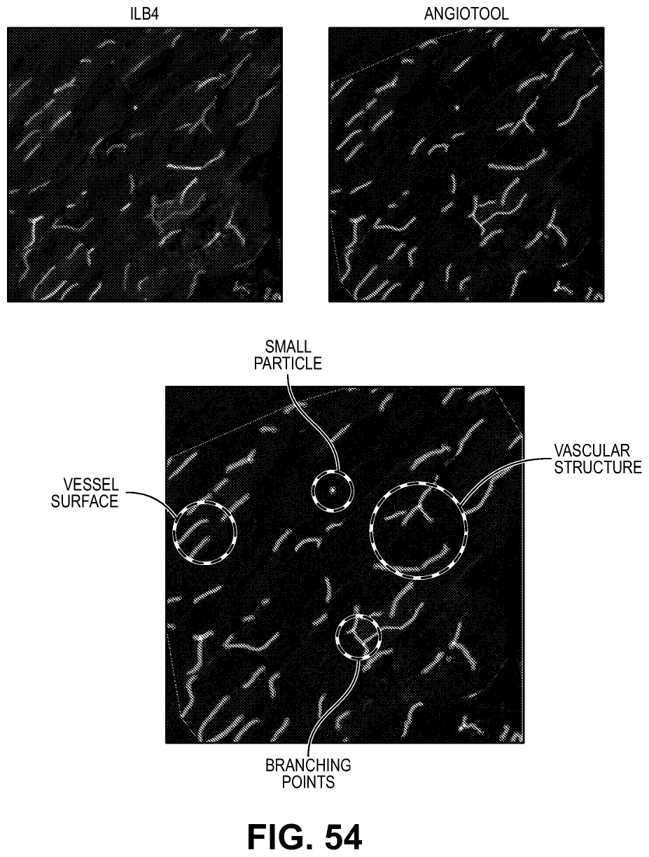

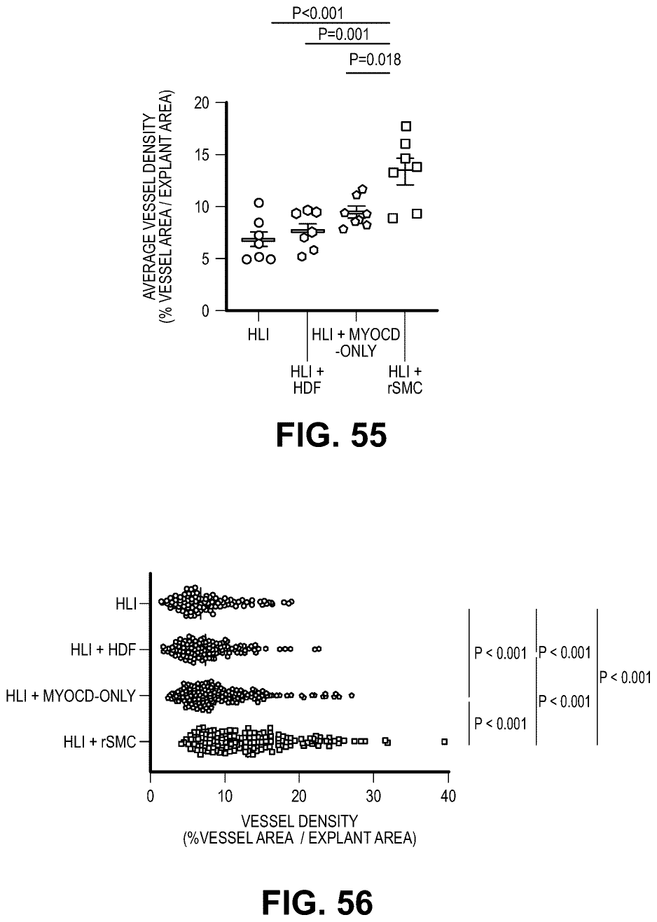

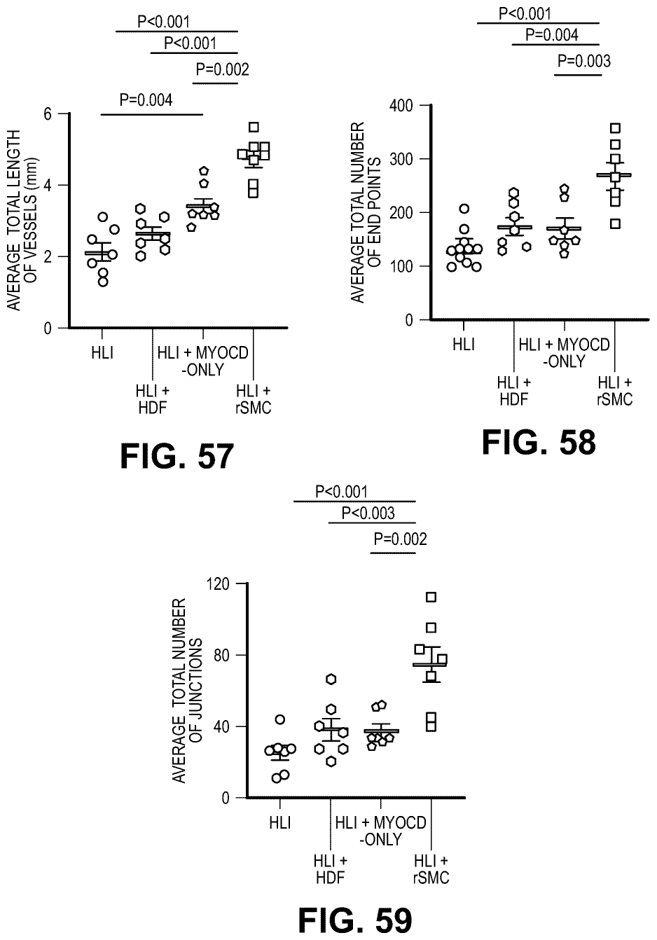

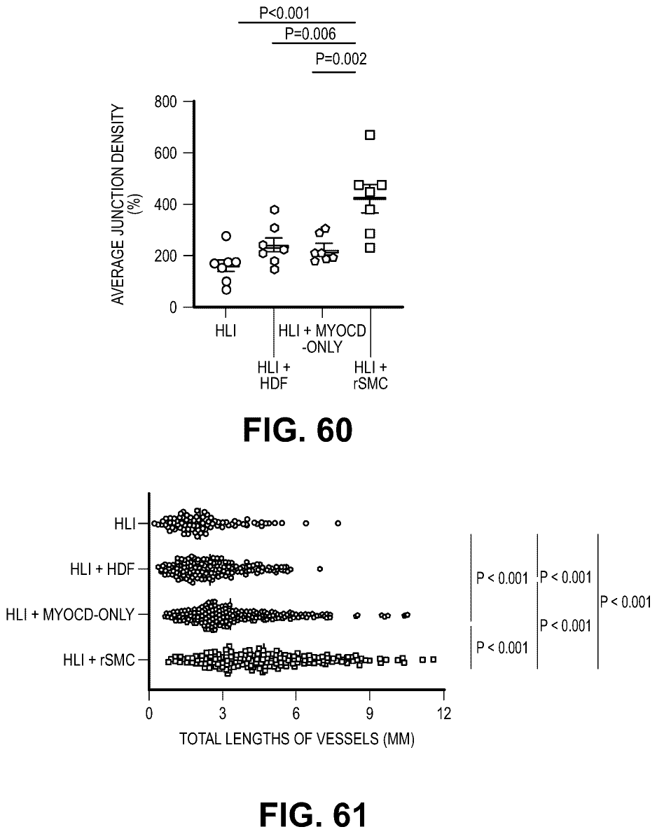

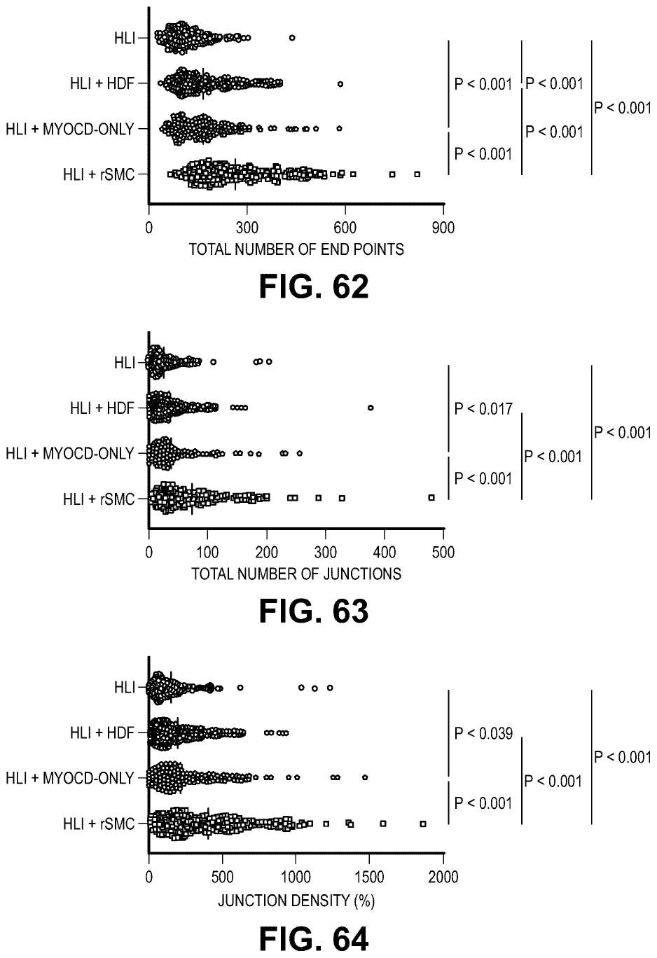

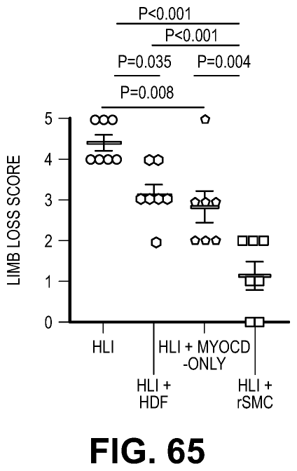

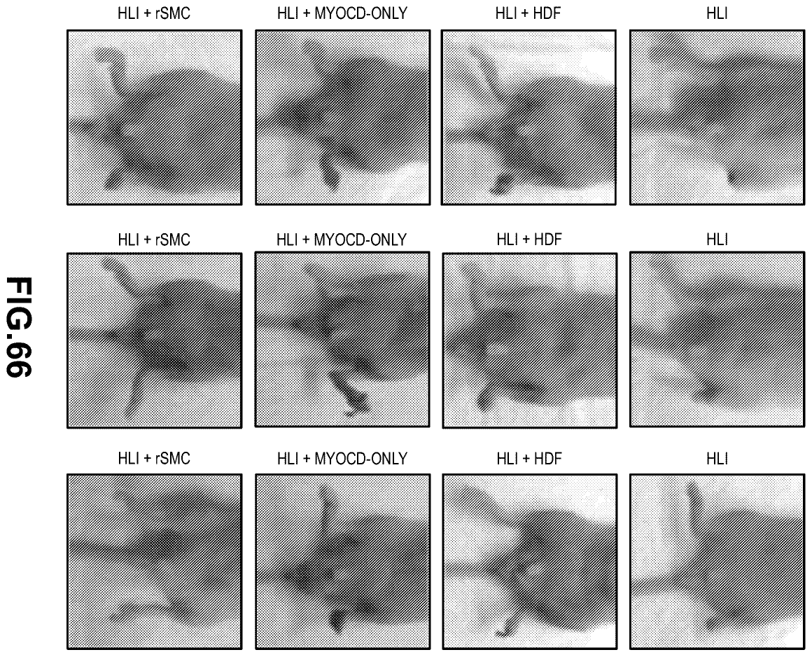

rSMCs can enhance recovery of hindlimb ischemia and promote neovascularization. To test whether rSMCs can promote recovery of tissue ischemia, a murine model of hindlimb ischemia was used. After ligating femoral vessels, HDFs, MYOCD-only cells or rSMCs were directly injected into the ischemic thigh muscle. As shown in FIG. 52, serial analysis of blood perfusion by laser Doppler perfusion imaging (LDPI) demonstrated significantly enhanced flow recovery in the rSMC group compared to HDF and MYOCD-only groups. This was evident 14 days after induction of ischemia and persisted over day 28 (FIG. 52). We sacrificed the mice at day 28 and harvested the muscle following injection of a fluorescein conjugated Griffonia simplicifolia isolectin B4 (ILB4) into the heart for systemic vascular perfusion. The density of ILB4+ vessels in the hindlimb muscle was significantly higher in the rSMC group compared to the other groups (HLI, HDF, and MYOCD-only) (FIG. 53). To analyze the vascular networks, confocal images of the ILB4+ vessels were evaluated by using AngioTool. These ILB4+ vessels and their branching points were labelled (FIG. 54). Morphometric parameters were then computed including the vessel density, vessel length, the number of end points and junctions, and junction density. As shown in FIGS. 55-64, all of these parameters were significantly higher in the rSMC group compared to the other groups. These results indicate that the injection of rSMCs not only increased the vessel number, but also increased their network formation. In addition, the rSMC group showed a significantly lower limb loss score (FIGS. 65-66). Taken together, these results indicate that rSMCs enhanced recovery of hindlimb ischemia and promoted functional and structural neovascularization.

### Example 5: RSMCs Contribution to Microvessel Formation

### Materials and Methods

Histological analysis. Hindlimb ischemia and cell transplantation were performed as described above. Cells were prelabeled with CellTracker™ CM-DiI before injection into ischemic hindlimb tissue. Twenty-eight days after injection, before euthanasia, mice (Japan SLC, Inc.) were systemically perfused with fluorescein-conjugated ILB4 (Vector Laboratories, Inc.) to identify functional endothelium. Mouse ischemic hindlimb tissues were removed, fixed in 4% PFA (VWR) overnight at 4° C., and incubated in 30% sucrose (Sigma-Aldrich) solution overnight at 4° C. in the dark. The ischemic hindlimb tissues were then subject to tissue section. The tissue sections were washed with DPBS (Corning), fixed with 4% PFA in the dark for half an hour at RT, and permeabilized with 1-3% Triton X-100 (Sigma-Aldrich) in DPBS in the dark for an hour at RT. The permeabilized tissue sections were incubated with blocking buffer containing 0.5-1% Triton X-100 and 1% BSA in DPBS in the dark for three hours at RT. The tissue sections were incubated with non-conjugated primary antibodies overnight at 4° C. in the dark, followed by fluorochrome-labelled secondary antibodies for three hours at 4° C. in the dark, according to the manufacturer's instructions. Primary and secondary antibodies are shown in Table 2. The tissue sections were counter-stained with DAPI (Invitrogen) to visualize the nuclei. Contribution of transplanted cells to neovascularization was captured by a Zeiss LSM 700 or 780 confocal microscope (Carl Zeiss AG).

### Results

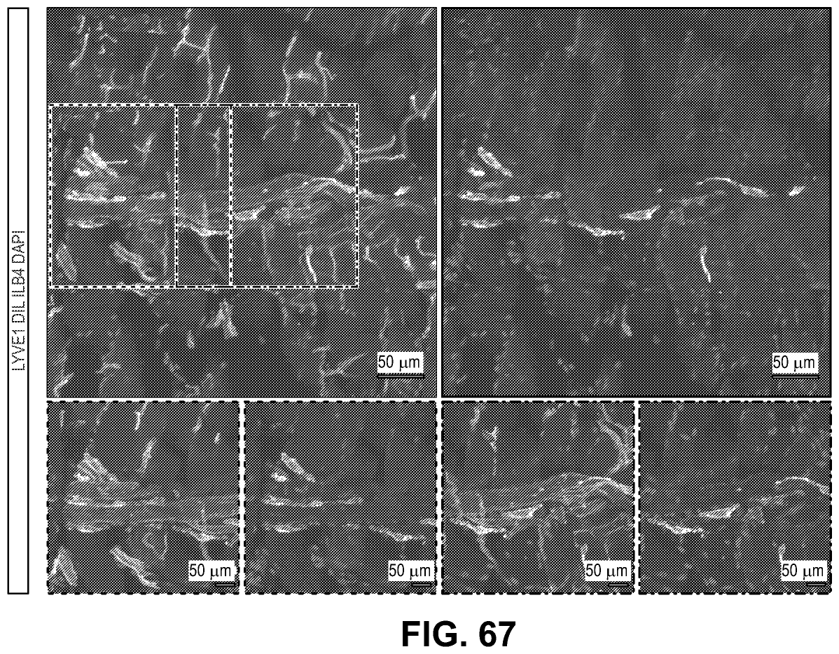

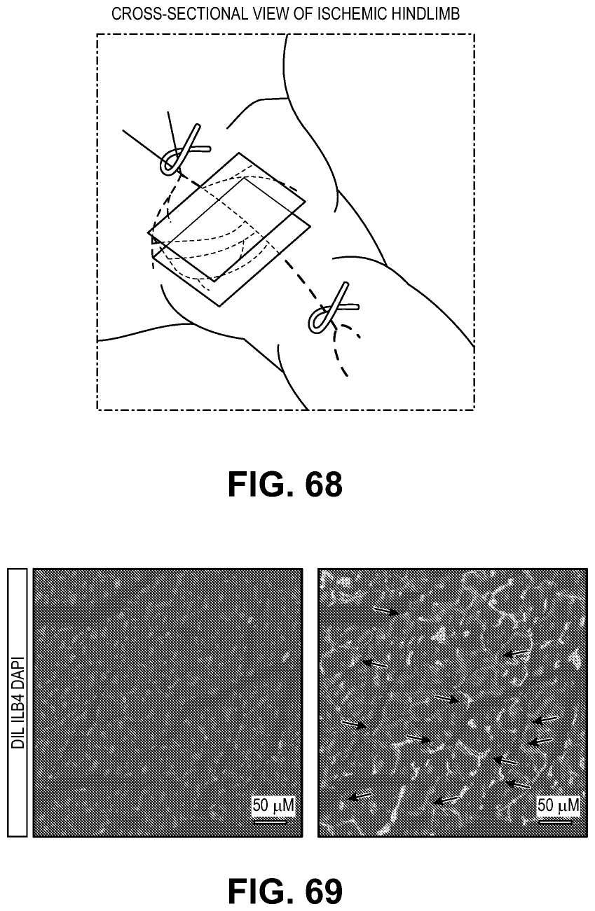

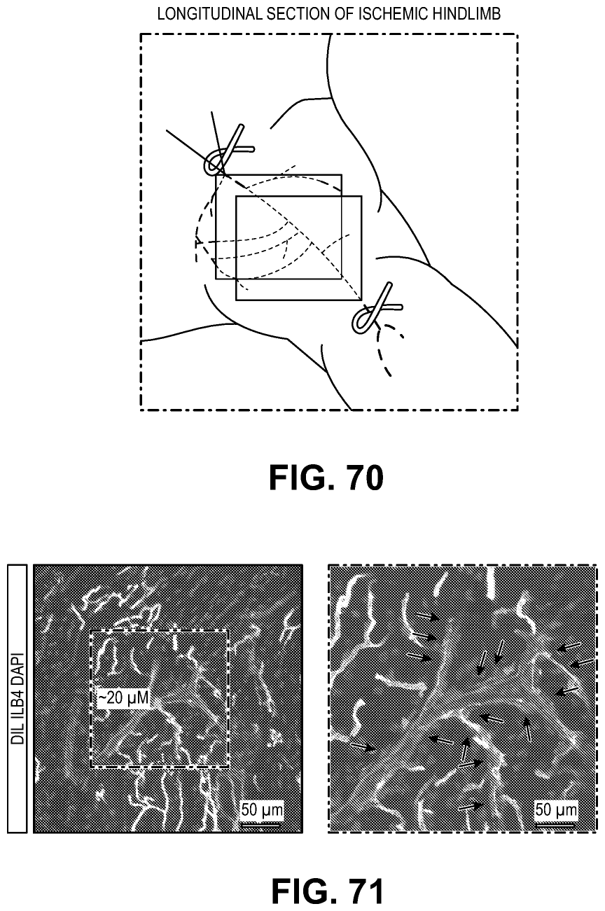

rSMCs contribute to microvessel formation through pericytic investment. To investigate the behaviors of transplanted rSMCs in vivo, rSMCs were pre-labelled with a red fluorescence dye, CM-DiI. The DiI-prelabelled rSMCs were transplanted into three sites of ischemic hindlimbs. Twenty-eight days after the transplantation, fluorescein-conjugated-ILB4 was systemically injected to identity functional endothelium (FIG. 67). The distributions of engrafted rSMCs in relation to the ILB4+ blood vessels at various levels of the vascular trees were determined. As shown in FIG. 68, a cross-sectional of the cell-injected ischemic hindlimb muscle was visualized. This view showed a large proportion of engrafted rSMCs (arrows) were localized close to the capillary-sized vessels (<~10.0 μm in diameter) (FIG. 69). These findings imply that rSMCs can contribute to vessel formation through pericytic investment. As shown in FIG. 70, longitudinal sections of the cell-injected ischemic hindlimb muscle were also visualized. In the longitudinal sections, rSMCs (arrows) were localized to vessels with diameters of >20 μm (FIG. 71).

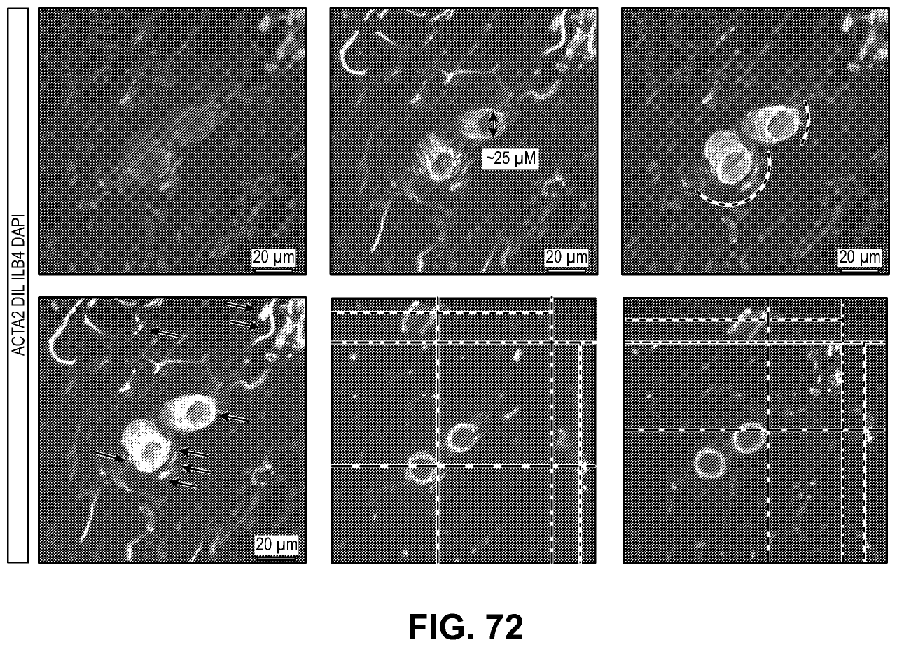

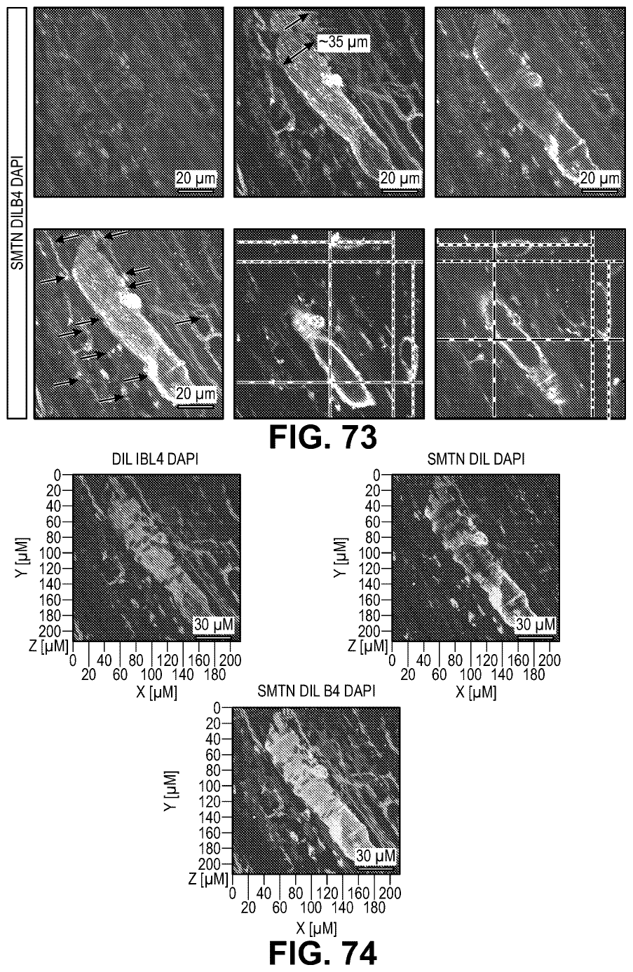

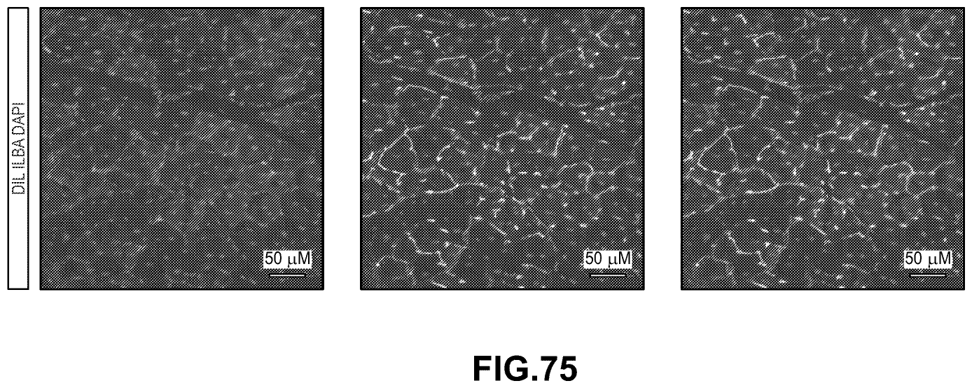

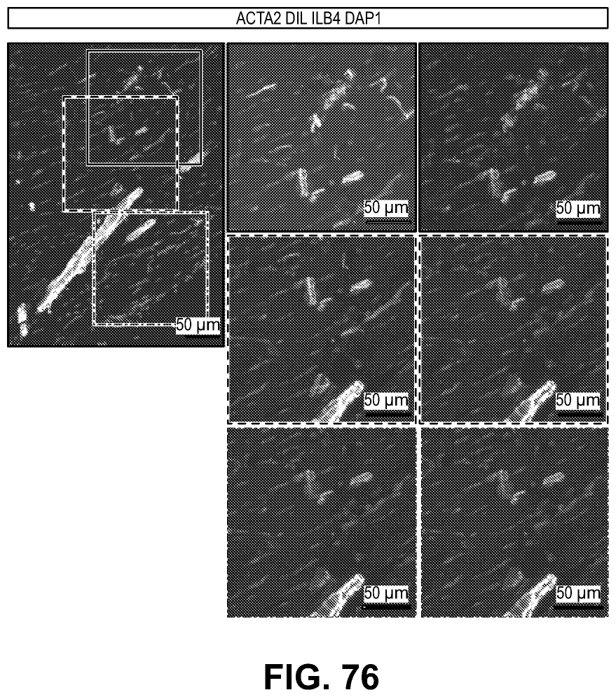

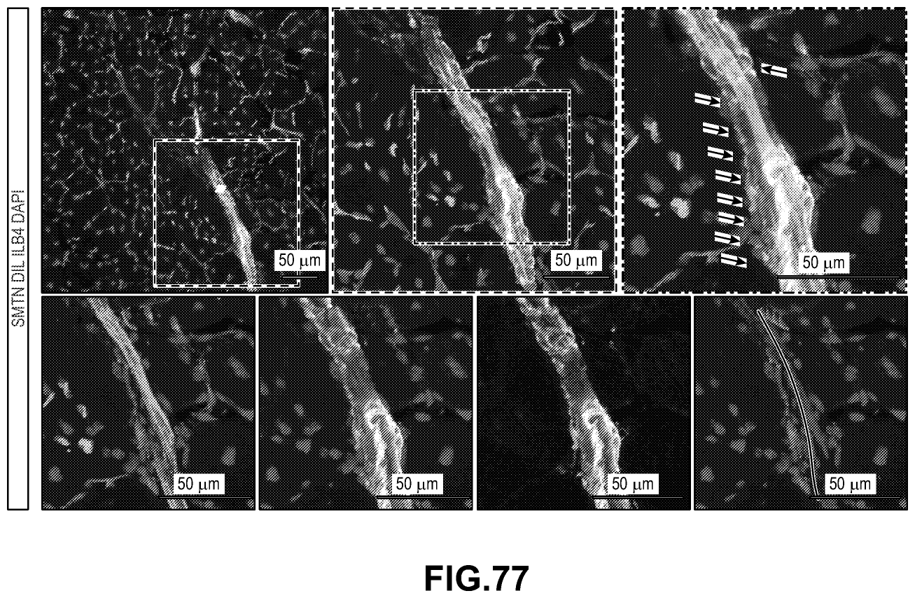

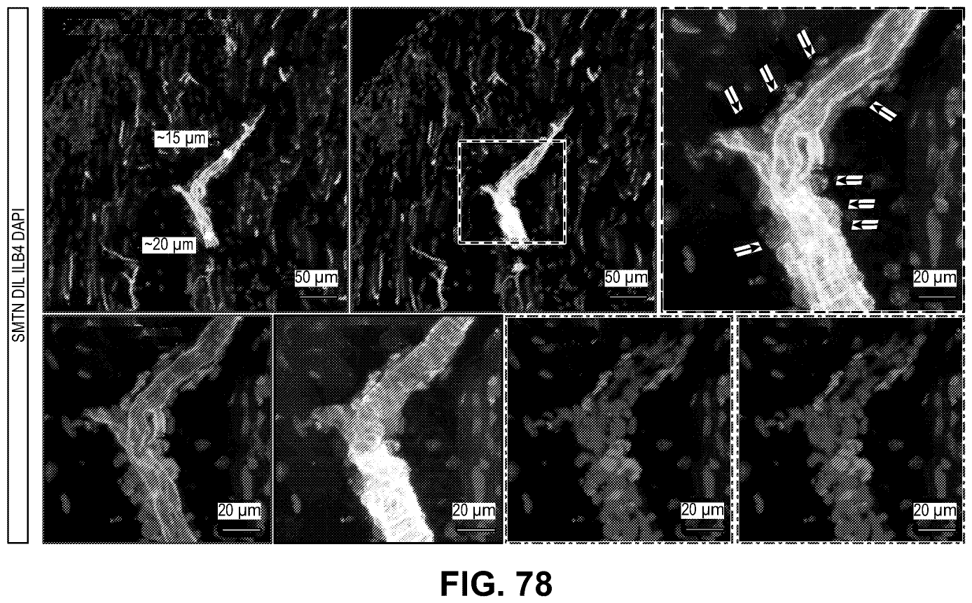

rSMCs contribute to microvessel formation as vascular SMCs. Immunostaining for ACTA2 further showed that a fraction of the ACTA2-expressing rSMCs (ACTA2+DiI+, arrows) formed a narrow circumferential band surrounding the vessels with ~25 μm in diameter (curved dashed line) (FIG. 72). These findings imply that rSMCs can contribute to the smooth muscle cell layer of larger microvessels. To verify the identity of the rSMCs as vascular SMCs in vivo, immunostaining for SMTN, a mature, contractile SMC marker (see, for example, Owens et al., Physiological Rev., 84:767-801 (2004) and van der Loop et al., Arterioscler. Thomb. Vasc. Biol., 17:665-671 (1997)) was performed. Confocal microscopic examination showed that DiI+ rSMCs expressed SMTN and were localized in vascular walls of vessels ~10 to 35 μm in diameter (FIGS. 73-74/77-78). Some rSMCs clearly contributed to the smooth muscle layer of the wall, indicating their contribution as vascular SMCs. Because transplanted rSMCs were heterogenous including a mixed population of HDFs and rSMCs, the fate of DiI+HDFs, which were similarly injected into the hindlimb muscle were examined to compare the results obtained above (FIGS. 75-76). Confocal microscopic examination showed that HDFs were much less localized to the capillary-sized vessels and rarely found at the relatively larger vessels that were stained for ACTA2 (FIGS. 75-76). These findings suggest that HDFs seemed to contribute to vessel formation at a minimal level. Together, these findings show that transplanted rSMCs can contribute to vessel formation through pericytes in capillary-sized vessels and vascular SMCs in larger blood vessels.

### Example 6: RSMCs Effect on Vascular Permeability

### Materials and Methods

In vitro vascular permeability assay. Cells were seeded onto the abluminal side of a 24-well Transwell® insert with 0.4 μm pore size (Corning) at a density of 2.0×10⁵ cells per ml and incubated for four hours at 37° C. with 5% CO₂. Human umbilical vein ECs (HUVECs) were seeded onto the luminal side of the insert at a density of 1.0×10⁵ cells per ml and incubated overnight at 37° C. with 5% CO₂. The inserts were transferred to fresh EGM-2 medium, carefully replaced with EGM-2 medium containing one mg per ml of FITC-dextran (Sigma-Aldrich), and then incubated for four hours at 37° C. with 5% CO₂. The cells were harvested, and the intensity of FITC-dextran was determined by Varioskan Flash spectral scanning multimode reader (Thermo Fisher Scientific), with the green fluorescence in excitation and emission at 485 and 535 nm, respectively.

### Results

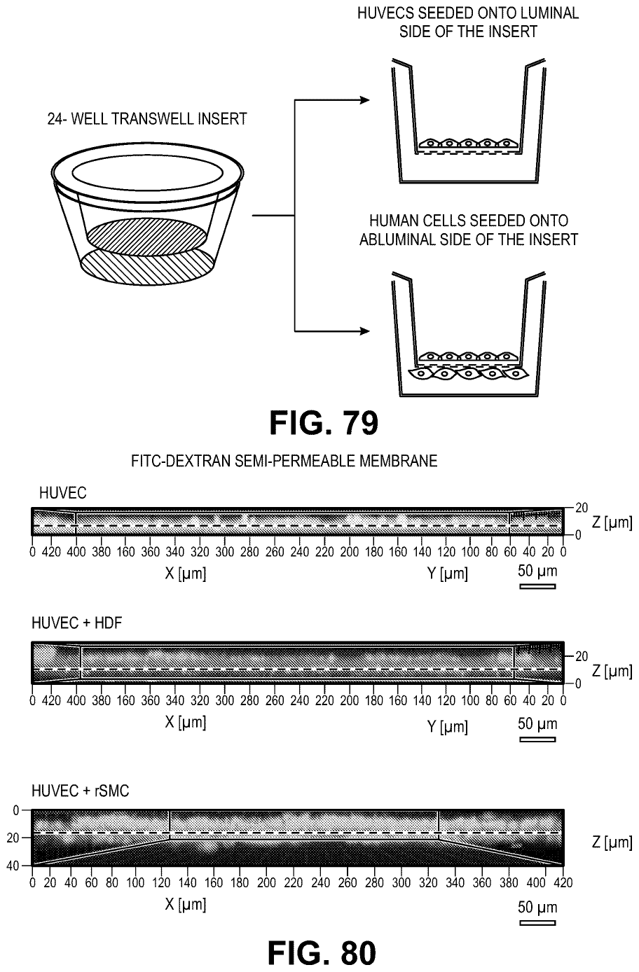

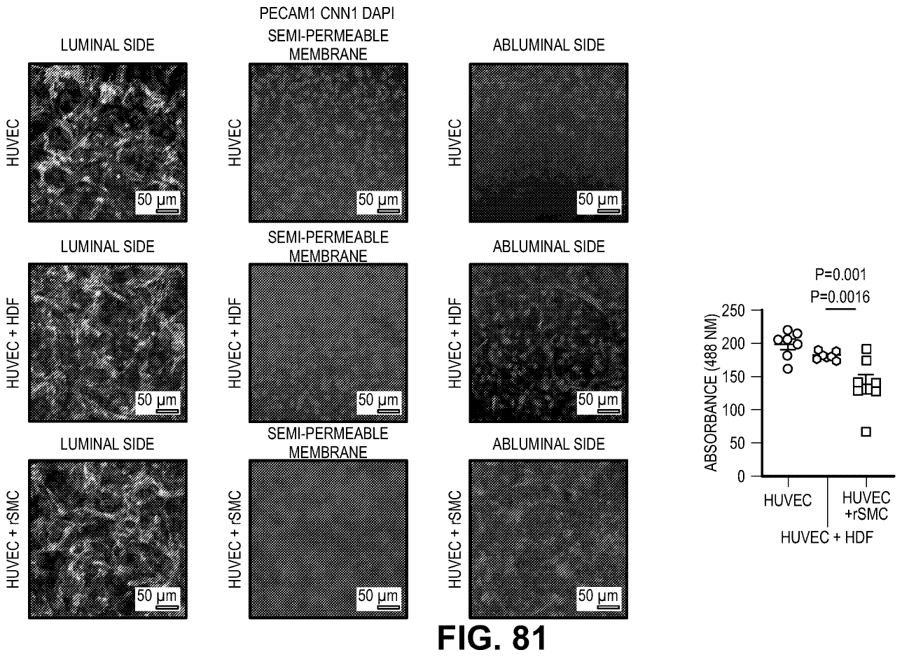

rSMCs can restrict vascular permeability. Ensheathment of mural cells over blood vessels tightly regulates vascular permeability restricting extravasation (see, for example, Armulik et al., Dev. Cell, 21:193-215 (2011); Armulik et al., Nature, 468:557-561 (2010)). To determine whether rSMCs can regulate vascular permeability, an in vitro co-culture model in which HUVECs and rSMCs were seeded onto opposite sides of a semi-porous membrane was employed (FIG. 79). FITC-dextran was then diffused through the membranes over time and the fluorescence intensity of the dextran in the lower chamber was measured (FIG. 80). The identity of HUVECs and rSMCs were verified by immunostaining for PECAM1 and CNN1, respectively (FIG. 81). Quantitatively, the total fluorescence (permeability) of FITC-dextran was significantly lower when HUVECs were co-cultured with rSMCs than cultured alone or co-cultured with HDFs. The results suggest that the presence of rSMCs forms a barrier to control vascular permeability.

### Example 7: Paracrine Effects of rSMCs on Ischemia

### Methods

Hindlimb ischemia, cell transplantation, and qRT-PCR. Induction of hindlimb ischemia and cell transplantation were performed as described above. qRT-PCR was also performed as previously described, using primers shown in Table 6.

TABLE 6
Primers used for qRT-PCR analysis in Example 7

| Gene | Forward | Reverse |
|---|---|---|
| Gapdh | CGTGTTCCTACCCCCAATGT | TGTCATCATACTTGGCAGGTTTCT |
| | (SEQ ID NO: 73) | (SEQ ID NO: 74) |
| Vegfa | CATCTTCAAGCCGTCCTGTGT | CAGGGCTTCATCGTTACAGCA |
| | (SEQ ID NO: 75) | (SEQ ID NO: 76) |
| Hif1a | CCTGCACTGAATCAAGAGGTTGC | CCATCAGAAGGACTTGCTGGCT |
| | (SEQ ID NO: 77) | (SEQ ID NO: 78) |
| Fgf2 | GTCACGGAAATACTCCAGTTGGT | CCGTTTTGGATCCGAGTTTATACT |
| | (SEQ ID NO: 79) | (SEQ ID NO: 80) |
| Igf1 | TGCTTCCGGAGCTGTGATCT | CGGGCTGCTTTTGTAGGCT |
| | (SEQ ID NO: 81) | (SEQ ID NO: 82) |
| Angpt1 | GGGACAGCAGGCAAACAGA | TGTCGTTATCAGCATCCTTCGT |
| | (SEQ ID NO: 83) | (SEQ ID NO: 84) |
| Ccr2 | ATCCACGGCATACTATCAACATC | CAAGGCTCACCATCATCGTAG |
| | (SEQ ID NO: 85) | (SEQ ID NO: 86) |
| Ccl2 | GTTGGCTCAGCCAGATGCA | AGCCTACTCATTGGGATCATCTTG |
| | (SEQ ID NO: 87) | (SEQ ID NO: 88) |
| Dll4 | GGAACCTTCTCACTCAACATCC | CTCGTCTGTTCGCCAAATCT |
| | (SEQ ID NO: 89) | (SEQ ID NO: 90) |
| Tgfb1 | CTCCCGTGGCTTCTAGTGC | GCCTTAGTTTGGACAGGATCTG |
| | (SEQ ID NO: 91) | (SEQ ID NO: 92) |
| Pdgfb | CATCCGCTCCTTTGATGATCTT | GTGCTCGGGTCATGTTCAAGT |
| | (SEQ ID NO: 93) | (SEQ ID NO: 94) |
| Hgf | ACTTCTGCCGGTCCTGTTG | CCCCTGTTCCTGATACACCT |
| | (SEQ ID NO: 95) | (SEQ ID NO: 96) |
| Csf1 | ATGAGCAGGAGTATTGCCAAGG | TCCATTCCCAATCATGTGGCTA |
| | (SEQ ID NO: 97) | (SEQ ID NO: 98) |
| Hey1 | CATGAAGAGAGCTCACCCAGA | CGCCGAACTCAAGTTTCC |
| | (SEQ ID NO: 99) | (SEQ ID NO: 100) |
| Mmp2 | CAAGTTCCCCGGCGATGTC | TTCTGGTCAAGGTCACCTGTC |
| | (SEQ ID NO: 101) | (SEQ ID NO: 102) |
| Mmp3 | CAAGTTCCCCGGCGATGTC | TTCTGGTCAAGGTCACCTGTC |
| | (SEQ ID NO: 103) | (SEQ ID NO: 104) |
| Mmp9 | CTGGACAGCCAGACACTAAAG | CTCGCGGCAAGTCTTCAGAG |
| | (SEQ ID NO: 105) | (SEQ ID NO: 106) |

### Results

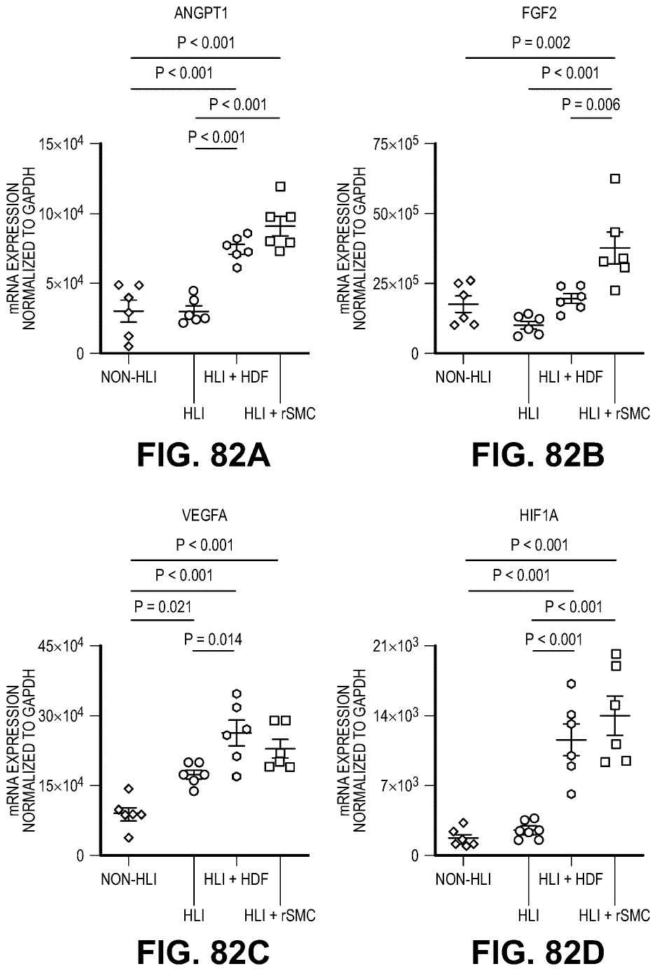

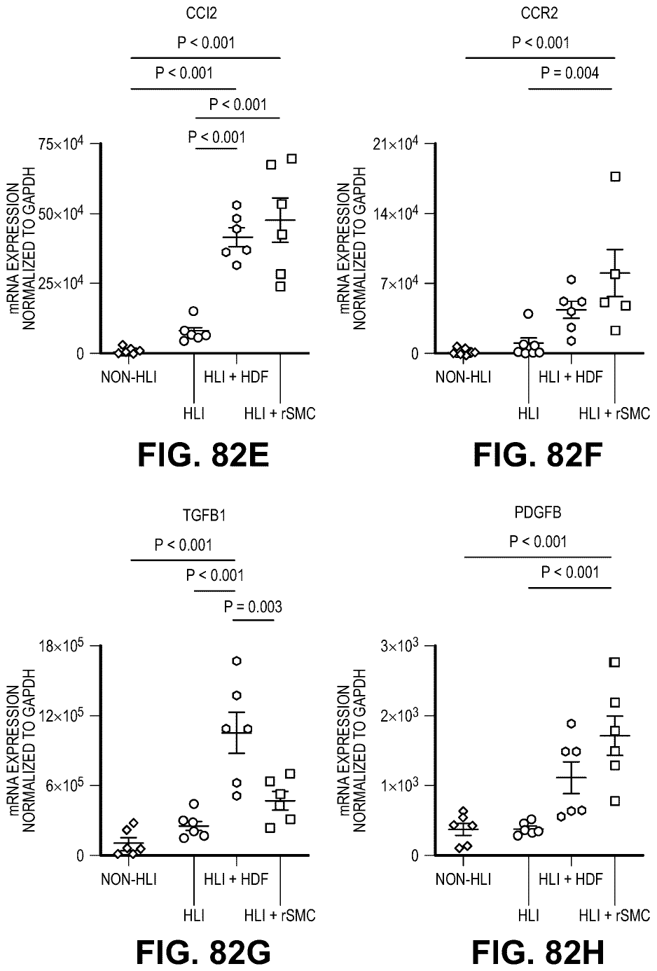

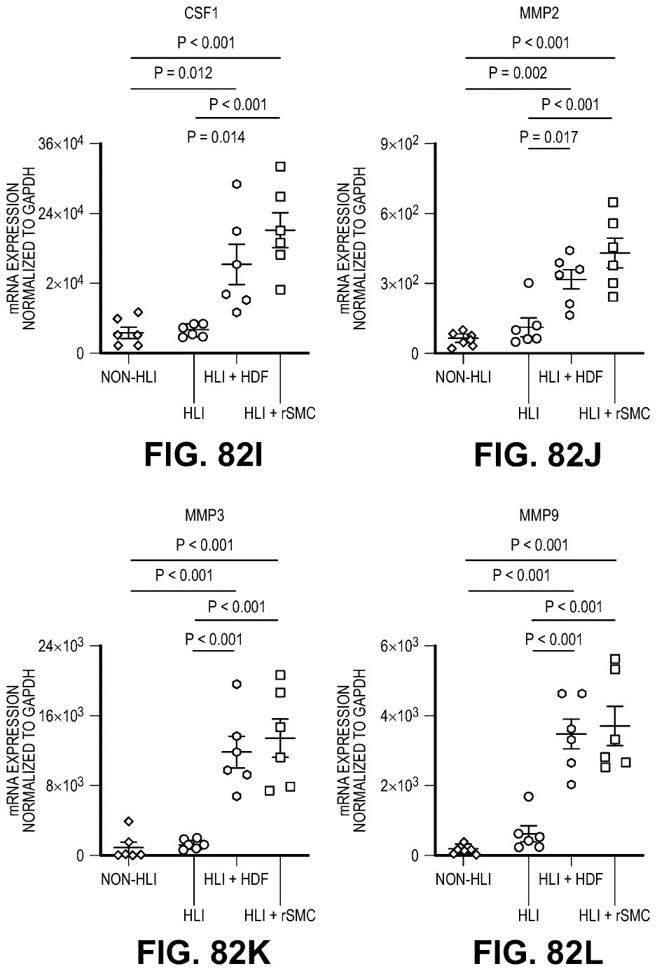

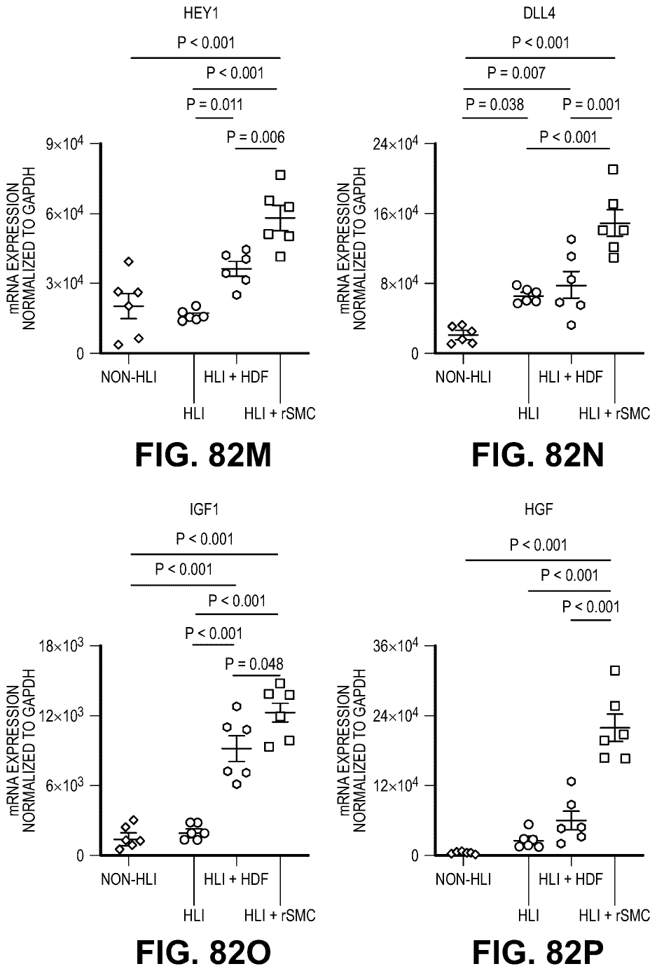

Angiogenic gene expression increased in HLI muscles treated with rSMCs. To gain insight into the mechanisms underlying the early therapeutic effects of rSMCs on hindlimb ischemia, qRT-PCR was conducted with muscles harvested at one-week post-surgery (FIG. 82). The expression of three representative angiogenic genes, Angpt1, Fgf2, and Vegfa (see, for example, Carmeliet, Nat. Med., 6:389-395 (2000)) was determined. The mRNA expression of Angpt1 and Fgf2 was greatly increased in the rSMC-HLI muscles compared to the HDF- and untreated HLI muscles. While the expression of Vegfa was surprisingly higher in the HDF group than in the rSMC group, that of Hif1a, an upstream regulator of Vegfa, was higher in the rSMC group. These results imply that both HDFs and rSMCs could exert paracrine effects to protect against ischemic injury.

Arteriogenic gene expression increased in HLI muscles treated with rSMCs. Next, as shown in FIG. 82, the expression of eight representative arteriogenic genes, Ccl2, its receptor Ccr2, Tgfb1, Pdgfb, Csf1, and Mmps (2, 3, and 9) were determined (see, for example, Cai et al., Am. J. Heart Circ. Physiol., 284: H31-40 (2003) and Heil & Schaper, Circ. Res., 95:449-458 (2004).). With the exception of Tgfb1, the mRNA expression of these arteriogenic genes was higher in the rSMC-HLI muscles than the HDF- and untreated HLI muscles (FIG. 82). In addition, as shown in FIG. 82, the expression of two members of the Notch signaling pathway (Hey1 and Dll4), was examined, along with Igf1 and Hgf. The two members of the Notch signaling pathways, Hey1 and Dll4, have a prominent place in vessel stabilization and maturation (see, for example, Hoglund & Majesky, Circulation, 125:212-215 (2012); Manderfield et al., Circulation, 125:314-323 (2012); and Scheppke et al., Blood, 119:2149-2158 (2012)), while Igf1 and Hgf play a critical role in improvement of SMC engraftment and vascular function (see, for example, Liu et al., Am. J. Physiol. Heart Circ. Physiol., 287: H2840-2849 (2004) and Powell et al., Circulation, 118:58-65 (2008)). The rSMC group showed a marked increase in all four gene expression levels compared to the HDF group (FIG. 82). These findings suggest that rSMC transplantation increased not only angiogenic but arteriogenic and vessel-stabilizing factors in ischemic limbs.

1. A method of producing a reprogrammed smooth muscle cell, comprising culturing a fibroblast with an all-trans-retinoic acid (ATRA) under conditions that produce a reprogrammed smooth muscle cell from the fibroblast, wherein the fibroblasts are genetically modified to overexpress myocardin.

2. The method of claim 1, wherein the conditions that generate the reprogrammed smooth muscle cell from the fibroblast comprise contacting the fibroblast with the ATRA for at least two days.

3. The method of claim 2, wherein the contacting step is for 4-8 days.

4. The method of claim 2 or 3, wherein the contacting step comprises contacting the fibroblast with ATRA.

5. The method of claim 1, wherein the fibroblast is a mammalian fibroblast.

6. The method of claim 5, wherein the fibroblast is a human fibroblast.

7. The method of claim 6, wherein the human fibroblast is a human dermal fibroblast.

8. The method of claim 1, further comprising genetically modifying the fibroblast by introducing into the fibroblast a heterologous nucleic acid that encodes myocardin.

9. The method of claim 8, wherein the heterologous nucleic acid is introduced into the fibroblast by viral transduction.

10. The method of claim 8, wherein the heterologous nucleic acid is stably integrated the fibroblast genome.

11. The method of claim 8, wherein the heterologous nucleic acid is introduced into the fibroblast by gene editing.

12. A reprogrammed smooth muscle cell prepared by the method of claim 1.

13. A reprogrammed smooth muscle cell comprising a heterologous nucleic acid encoding myocardin, wherein the reprogrammed smooth muscle cell co-expresses CNN1 and SMTN in a non-striated pattern.

14. The reprogrammed smooth muscle cell of claim 12 or 13, wherein the reprogrammed smooth muscle cells contract by more than 10% in the presence of carbachol.

15. A composition comprising a population of reprogrammed smooth muscle cells according to claim 12 and a pharmaceutically acceptable carrier.

16. A method of treating a subject with ischemia or at risk of developing ischemia, the method comprising administering to the subject an effective amount of the composition of claim 15.

17. The method of claim 16, wherein the effective amount of the composition increases vascular perfusion in the subject.

18. The method of claim 16, wherein the effective amount of the composition increases neovascularization in the subject.

19. The method of claim 16, wherein the effective amount of the composition increases arteriogenesis in the subject.


SEQUENCE LISTING (106 sequences)

SEQ ID NO: 1 - DNA, 23 bp, synthetic construct
  source 1..23: mol_type = other DNA
ggtggtctcctctgacttcaaca

SEQ ID NO: 2 - DNA, 19 bp, synthetic construct
  source 1..19: mol_type = other DNA
gtggtcgttgagggcaatg

SEQ ID NO: 3 - DNA, 19 bp, synthetic construct
  source 1..19: mol_type = other DNA
agaccaggtcccacactga

SEQ ID NO: 4 - DNA, 24 bp, synthetic construct
  source 1..24: mol_type = other DNA
ttcataatagccaaaggcataaaa

SEQ ID NO: 5 - DNA, 20 bp, synthetic construct
  source 1..20: mol_type = other DNA
actcctggggtctgagcatt

SEQ ID NO: 6 - DNA, 20 bp, synthetic construct
  source 1..20: mol_type = other DNA
ctggacgtttcagtggtggt

SEQ ID NO: 7 - DNA, 20 bp, synthetic construct
  source 1..20: mol_type = other DNA
ttacctctcgtggctttggc

SEQ ID NO: 8 - DNA, 21 bp, synthetic construct
  source 1..21: mol_type = other DNA
ccagcaagctttgtcaagacc

SEQ ID NO: 9 - DNA, 21 bp, synthetic construct
  source 1..21: mol_type = other DNA
aaaagacagctacgtgggtga

SEQ ID NO: 10 - DNA, 22 bp, synthetic construct
  source 1..22: mol_type = other DNA
gccatgttctatcgggtacttc

SEQ ID NO: 11 - DNA, 22 bp, synthetic construct
  source 1..22: mol_type = other DNA
agtgcagtccaaaatcgagaag

SEQ ID NO: 12 - DNA, 20 bp, synthetic construct
  source 1..20: mol_type = other DNA
cttgctcagaatcacgccat

SEQ ID NO: 13 - DNA, 22 bp, synthetic construct
  source 1..22: mol_type = other DNA
ctgttcctgctgaaggtgtacg

SEQ ID NO: 14 - DNA, 23 bp, synthetic construct
  source 1..23: mol_type = other DNA
cctaccttcaagccagcagtttc

SEQ ID NO: 15 - DNA, 20 bp, synthetic construct
  source 1..20: mol_type = other DNA
gaaatccggcacctcaagga

SEQ ID NO: 16 - DNA, 20 bp, synthetic construct
  source 1..20: mol_type = other DNA
gatggggagattgatccggc

SEQ ID NO: 17 - DNA, 21 bp, synthetic construct
  source 1..21: mol_type = other DNA
gtcaacccaaaattggcacca

SEQ ID NO: 18 - DNA, 22 bp, synthetic construct
  source 1..22: mol_type = other DNA
accttgtttcctttcgtcttcg

SEQ ID NO: 19 - DNA, 24 bp, synthetic construct
  source 1..24: mol_type = other DNA
agaagccagggagaaggaaaccaa

SEQ ID NO: 20 - DNA, 24 bp, synthetic construct
  source 1..24: mol_type = other DNA
tggagctgaccaggtcttccattt

SEQ ID NO: 21 - DNA, 20 bp, synthetic construct
  source 1..20: mol_type = other DNA
ccttctcatctgcggagacc

SEQ ID NO: 22 - DNA, 20 bp, synthetic construct
  source 1..20: mol_type = other DNA
cagtccaccagcatcttcca

SEQ ID NO: 23 - DNA, 20 bp, synthetic construct
  source 1..20: mol_type = other DNA
ccgctgaggtgggtataggt

SEQ ID NO: 24 - DNA, 20 bp, synthetic construct
  source 1..20: mol_type = other DNA
cttcccgatgcaaggactga

SEQ ID NO: 25 - DNA, 21 bp, synthetic construct
  source 1..21: mol_type = other DNA
ggcaccgaagatgaactggac

SEQ ID NO: 26 - DNA, 22 bp, synthetic construct
  source 1..22: mol_type = other DNA
gcgtctgttcagagaagctacg

SEQ ID NO: 27 - DNA, 22 bp, synthetic construct
  source 1..22: mol_type = other DNA
gagaggtctgtggcaaagttgg

SEQ ID NO: 28 - DNA, 22 bp, synthetic construct
  source 1..22: mol_type = other DNA
ggaggtgatgtcattgagtgcg

SEQ ID NO: 29 - DNA, 21 bp, synthetic construct
  source 1..21: mol_type = other DNA
gtgcttctgagatgtgggctt

SEQ ID NO: 30 - DNA, 20 bp, synthetic construct
  source 1..20: mol_type = other DNA
gggtcagcagctcctttagt

SEQ ID NO: 31 - DNA, 20 bp, synthetic construct
  source 1..20: mol_type = other DNA
ctgagcacacataccgctcc

SEQ ID NO: 32 - DNA, 20 bp, synthetic construct
  source 1..20: mol_type = other DNA
gccctcacacttgaccagtt

SEQ ID NO: 33 - DNA, 20 bp, synthetic construct
  source 1..20: mol_type = other DNA
gtccgcacattcgagcaaag

SEQ ID NO: 34 - DNA, 20 bp, synthetic construct
  source 1..20: mol_type = other DNA
cgctgctagttctcagtgct

SEQ ID NO: 35 - DNA, 22 bp, synthetic construct
  source 1..22: mol_type = other DNA
tttttgtgacggtcttggaagt

SEQ ID NO: 36 - DNA, 23 bp, synthetic construct
  source 1..23: mol_type = other DNA
tgtctgagtgtggttgtaatagc

SEQ ID NO: 37 - DNA, 23 bp, synthetic construct
  source 1..23: mol_type = other DNA
tggttttgaggcagctagtatca

SEQ ID NO: 38 - DNA, 23 bp, synthetic construct
  source 1..23: mol_type = other DNA
agtcctgaatagtagcgatcctt

SEQ ID NO: 39 - DNA, 23 bp, synthetic construct
  source 1..23: mol_type = other DNA
cgaggtgatagtgtggtttatgg

SEQ ID NO: 40 - DNA, 23 bp, synthetic construct
  source 1..23: mol_type = other DNA
gcaccattcaactcctcgctttc

SEQ ID NO: 41 - DNA, 22 bp, synthetic construct
  source 1..22: mol_type = other DNA
agcgagtggatgccgcctttaa

SEQ ID NO: 42 - DNA, 22 bp, synthetic construct
  source 1..22: mol_type = other DNA
cattccaggcatctgcgatgag

SEQ ID NO: 43 - DNA, 22 bp, synthetic construct
  source 1..22: mol_type = other DNA
gtcctgcctgtcaatgaccaac

SEQ ID NO: 44 - DNA, 22 bp, synthetic construct
  source 1..22: mol_type = other DNA
cgatggtgtagaccagatcctc

SEQ ID NO: 45 - DNA, 20 bp, synthetic construct
  source 1..20: mol_type = other DNA
gggcccaattacccatcctt

SEQ ID NO: 46 - DNA, 20 bp, synthetic construct
  source 1..20: mol_type = other DNA
gaaggtggggtgagcatcat

SEQ ID NO: 47 - DNA, 21 bp, synthetic construct
  source 1..21: mol_type = other DNA
cgagatggagatcggtatggt

SEQ ID NO: 48 - DNA, 20 bp, synthetic construct
  source 1..20: mol_type = other DNA
gggtcttcttacccggcttg

SEQ ID NO: 49 - DNA, 21 bp, synthetic construct
  source 1..21: mol_type = other DNA
caaacggaagattcgttcccg

SEQ ID NO: 50 - DNA, 21 bp, synthetic construct
  source 1..21: mol_type = other DNA
ttgaggtcatcggctagtctg

SEQ ID NO: 51 - DNA, 23 bp, synthetic construct
  source 1..23: mol_type = other DNA
cagatcgttctgaacttgtcagg

SEQ ID NO: 52 - DNA, 23 bp, synthetic construct
  source 1..23: mol_type = other DNA
cccaacgcctataattgcttctt

SEQ ID NO: 53 - DNA, 21 bp, synthetic construct
  source 1..21: mol_type = other DNA
cctgcctgaagtgcgagaaat

SEQ ID NO: 54 - DNA, 20 bp, synthetic construct
  source 1..20: mol_type = other DNA
cctttaggcccaaacatggc

SEQ ID NO: 55 - DNA, 19 bp, synthetic construct
  source 1..19: mol_type = other DNA
cggcagcggactcacttta

SEQ ID NO: 56 - DNA, 20 bp, synthetic construct
  source 1..20: mol_type = other DNA
gttggtccacacagcgattt

SEQ ID NO: 57 - DNA, 21 bp, synthetic construct
  source 1..21: mol_type = other DNA
gagggccaagacgaagacatc

SEQ ID NO: 58 - DNA, 21 bp, synthetic construct
  source 1..21: mol_type = other DNA
cagatcacgtcatcgcacaac

SEQ ID NO: 59 - DNA, 20 bp, synthetic construct
  source 1..20: mol_type = other DNA
ggccctcaaggtttccaagg

SEQ ID NO: 60 - DNA, 21 bp, synthetic construct
  source 1..21: mol_type = other DNA
caccctgtggtccaacaactc

SEQ ID NO: 61 - DNA, 19 bp, synthetic construct
  source 1..19: mol_type = other DNA
gcccggatgtcgcttacag

SEQ ID NO: 62 - DNA, 21 bp, synthetic construct
  source 1..21: mol_type = other DNA
aaatgcagacgcagggtacag

SEQ ID NO: 63 - DNA, 23 bp, synthetic construct
  source 1..23: mol_type = other DNA
ggattggtaaacctgaccttcat

SEQ ID NO: 64 - DNA, 23 bp, synthetic construct
  source 1..23: mol_type = other DNA
gataaacgcagatactgcaatgc

SEQ ID NO: 65 - DNA, 22 bp, synthetic construct
  source 1..22: mol_type = other DNA
ggctgctatgacacctacaatg

SEQ ID NO: 66 - DNA, 23 bp, synthetic construct
  source 1..23: mol_type = other DNA
gtagtgaatgttgcatctcacca

SEQ ID NO: 67 - DNA, 21 bp, synthetic construct
  source 1..21: mol_type = other DNA
gcgatacgacattgaggccaa

SEQ ID NO: 68 - DNA, 19 bp, synthetic construct
  source 1..19: mol_type = other DNA
ttcccacggaggtccatca

SEQ ID NO: 69 - DNA, 21 bp, synthetic construct
  source 1..21: mol_type = other DNA
tttgaccttcgaggcaagttt

SEQ ID NO: 70 - DNA, 21 bp, synthetic construct
  source 1..21: mol_type = other DNA
tgcagagatcctcactctccg

SEQ ID NO: 71 - DNA, 2962 bp, Homo sapiens
  source 1..2962: mol_type = genomic DNA
caacatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtgg

SEQ ID NO: 72 - protein, 986 aa, Homo sapiens
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 73 - DNA, 20 bp, synthetic construct
  source 1..20: mol_type = other DNA
cgtgttcctacccccaatgt

SEQ ID NO: 74 - DNA, 24 bp, synthetic construct
  source 1..24: mol_type = other DNA
tgtcatcatacttggcaggtttct

SEQ ID NO: 75 - DNA, 21 bp, synthetic construct
  source 1..21: mol_type = other DNA
catcttcaagccgtcctgtgt

SEQ ID NO: 76 - DNA, 21 bp, synthetic construct
  source 1..21: mol_type = other DNA
cagggcttcatcgttacagca

SEQ ID NO: 77 - DNA, 23 bp, synthetic construct
  source 1..23: mol_type = other DNA
cctgcactgaatcaagaggttgc

SEQ ID NO: 78 - DNA, 22 bp, synthetic construct
  source 1..22: mol_type = other DNA
ccatcagaaggacttgctggct

SEQ ID NO: 79 - DNA, 23 bp, synthetic construct
  source 1..23: mol_type = other DNA
gtcacggaaatactccagttggt

SEQ ID NO: 80 - DNA, 24 bp, synthetic construct
  source 1..24: mol_type = other DNA
ccgttttggatccgagtttatact

SEQ ID NO: 81 - DNA, 20 bp, synthetic construct
  source 1..20: mol_type = other DNA
tgcttccggagctgtgatct

SEQ ID NO: 82 - DNA, 19 bp, synthetic construct
  source 1..19: mol_type = other DNA
cgggctgcttttgtaggct

SEQ ID NO: 83 - DNA, 19 bp, synthetic construct
  source 1..19: mol_type = other DNA
gggacagcaggcaaacaga

SEQ ID NO: 84 - DNA, 22 bp, synthetic construct
  source 1..22: mol_type = other DNA
tgtcgttatcagcatccttcgt

SEQ ID NO: 85 - DNA, 23 bp, synthetic construct
  source 1..23: mol_type = other DNA
atccacggcatactatcaacatc

SEQ ID NO: 86 - DNA, 21 bp, synthetic construct
  source 1..21: mol_type = other DNA
caaggctcaccatcatcgtag

SEQ ID NO: 87 - DNA, 19 bp, synthetic construct
  source 1..19: mol_type = other DNA
gttggctcagccagatgca

SEQ ID NO: 88 - DNA, 24 bp, synthetic construct
  source 1..24: mol_type = other DNA
agcctactcattgggatcatcttg

SEQ ID NO: 89 - DNA, 22 bp, synthetic construct
  source 1..22: mol_type = other DNA
ggaaccttctcactcaacatcc

SEQ ID NO: 90 - DNA, 20 bp, synthetic construct
  source 1..20: mol_type = other DNA
ctcgtctgttcgccaaatct

SEQ ID NO: 91 - DNA, 19 bp, synthetic construct
  source 1..19: mol_type = other DNA
ctcccgtggcttctagtgc

SEQ ID NO: 92 - DNA, 22 bp, synthetic construct
  source 1..22: mol_type = other DNA
gccttagtttggacaggatctg

SEQ ID NO: 93 - DNA, 22 bp, synthetic construct
  source 1..22: mol_type = other DNA
catccgctcctttgatgatctt

SEQ ID NO: 94 - DNA, 21 bp, synthetic construct
  source 1..21: mol_type = other DNA
gtgctcgggtcatgttcaagt

SEQ ID NO: 95 - DNA, 19 bp, synthetic construct
  source 1..19: mol_type = other DNA
acttctgccggtcctgttg

SEQ ID NO: 96 - DNA, 20 bp, synthetic construct
  source 1..20: mol_type = other DNA
cccctgttcctgatacacct

SEQ ID NO: 97 - DNA, 22 bp, synthetic construct
  source 1..22: mol_type = other DNA
atgagcaggagtattgccaagg

SEQ ID NO: 98 - DNA, 22 bp, synthetic construct
  source 1..22: mol_type = other DNA
tccattcccaatcatgtggcta

SEQ ID NO: 99 - DNA, 21 bp, synthetic construct
  source 1..21: mol_type = other DNA
catgaagagagctcacccaga

SEQ ID NO: 100 - DNA, 18 bp, synthetic construct
  source 1..18: mol_type = other DNA
cgccgaactcaagtttcc

SEQ ID NO: 101 - DNA, 19 bp, synthetic construct
  source 1..19: mol_type = other DNA
caagttccccggcgatgtc

SEQ ID NO: 102 - DNA, 21 bp, synthetic construct
  source 1..21: mol_type = other DNA
ttctggtcaaggtcacctgtc

SEQ ID NO: 103 - DNA, 19 bp, synthetic construct
  source 1..19: mol_type = other DNA
caagttccccggcgatgtc

SEQ ID NO: 104 - DNA, 21 bp, synthetic construct
  source 1..21: mol_type = other DNA
ttctggtcaaggtcacctgtc

SEQ ID NO: 105 - DNA, 21 bp, synthetic construct
  source 1..21: mol_type = other DNA
ctggacagccagacactaaag

SEQ ID NO: 106 - DNA, 20 bp, synthetic construct
  source 1..20: mol_type = other DNA
ctcgcggcaagtcttcagag